SEQUENCE LISTING (68 sequences)

SEQ ID NO: 1 - protein, 114 aa, Candida albicans
AVGDVSEKTGTREVFFELNGEMRSVSVEDKTVSVELKTRPKASASNEVGAPMAGVVIEIRAHKHQQIAKGDPIAVLSAMKMEMVISAPCSGEIGDILIHEGDSVDANDLITSIH

SEQ ID NO: 2 - protein, 92 aa, Candida albicans
RSVSVEDKTVSVELKTRPKASASNEVGAPMAGVVIEIRAHKHQQIAKGDPIAVLSAMKMEMVISAPCSGEIGDILIHEGDSVDANDLITSIH

SEQ ID NO: 3 - protein, 73 aa, Candida albicans
ASASNEVGAPMAGVVIEIRAHKHQQIAKGDPIAVLSAMKMEMVISAPCSGEIGDILIHEGDSVDANDLITSIH

SEQ ID NO: 4 - protein, 124 aa, Drosophila melanogaster
KTLSVKALAVSADLKPNGIREVFFELNGQLRAVHILDKEAVKEIHVHPKANKSNKSEVGAPMPGTVIDIRVKVGDKVEKGQPLVVLSAMKMEMVVQSPLAGVVKKLEIANGTKLEGEDLIMIIE

SEQ ID NO: 5 - protein, 86 aa, Escherichia coli
EAPAAAEISGHIVRSPMVGTFYRTPSPDAKAFIEVGQKVNVGDTLCIVEAMKMMNQIEADKSGTVKAILVESGQPVEFDEPLVVIE

SEQ ID NO: 6 - protein, 14 aa, Escherichia coli
LGGIFEAMKMELRD

SEQ ID NO: 7 - protein, 21 aa, Escherichia coli
LFLHDFLNAQKVELYPVTSSG

SEQ ID NO: 8 - protein, 23 aa, Escherichia coli
MAGGLNDIFEAQKIEWHEDTGGS

SEQ ID NO: 9 - protein, 80 aa, Homo sapiens
HFHPKALKDVKGQIGAPMPGKVIDIKVVAGAKVAKGQPLCVLSAMKMETVVTSPMEGTVRKVHVTKDMTLEGDDLILEIE

SEQ ID NO: 10 - protein, 107 aa, Homo sapiens
GQRQVFFELNGQLRSILVKDTQAMKEMHFHPKALKDVKGQIGAPMPGKVIDIKVVAGAKVAKGQPLCVLSAMKMETVVTSPMEGTVRKVHVTKDMTLEGDDLILEIE

SEQ ID NO: 11 - protein, 80 aa, Homo sapiens; identical to SEQ ID NO: 9

SEQ ID NO: 12 - protein, 73 aa, Candida albicans; identical to SEQ ID NO: 3

SEQ ID NO: 13 - protein, 114 aa, Candida albicans; identical to SEQ ID NO: 1

SEQ ID NO: 14 - protein, 92 aa, Candida albicans; identical to SEQ ID NO: 2

SEQ ID NO: 15 - protein, 73 aa, Candida albicans; identical to SEQ ID NO: 3

SEQ ID NO: 16 - protein, 107 aa, Homo sapiens; identical to SEQ ID NO: 10

SEQ ID NO: 17 - protein, 80 aa, Homo sapiens; identical to SEQ ID NO: 9

SEQ ID NO: 18 - protein, 114 aa, Candida albicans; identical to SEQ ID NO: 1

SEQ ID NO: 19 - protein, 92 aa, Candida albicans; identical to SEQ ID NO: 2

SEQ ID NO: 20 - protein, 73 aa, Candida albicans; identical to SEQ ID NO: 3

SEQ ID NO: 21 - DNA, 38 bp, Artificial Sequence
  Synthetic primer sequence
atctacggatccaaagcttcagcatcaaatgaagttgg

SEQ ID NO: 22 - DNA, 43 bp, Artificial Sequence
  Synthetic primer sequence
atctacgaattcatcaatgaatactagtaatcaaatcattagc

SEQ ID NO: 23 - DNA, 34 bp, Artificial Sequence
  Synthetic primer sequence
atctacggatccatgagatcagtttccgttgaag

SEQ ID NO: 24 - DNA, 43 bp, Artificial Sequence; identical to SEQ ID NO: 22
  Synthetic primer sequence

SEQ ID NO: 25 - DNA, 28 bp, Artificial Sequence
  Synthetic primer sequence
ggatccatggctgttggtgatgtttcgg

SEQ ID NO: 26 - DNA, 43 bp, Artificial Sequence; identical to SEQ ID NO: 22
  Synthetic primer sequence

SEQ ID NO: 27 - DNA, 35 bp, Artificial Sequence
  Synthetic primer sequence
aattggtagaattcatgcacttccaccccaaggcc

SEQ ID NO: 28 - DNA, 42 bp, Artificial Sequence
  Synthetic primer sequence
gattaattctcgagttatcactcgatctccaggatgaggtcg

SEQ ID NO: 29 - DNA, 35 bp, Artificial Sequence
  Synthetic primer sequence
aattggtagaattcgccggccagaggcaggtcttc

SEQ ID NO: 30 - DNA, 42 bp, Artificial Sequence; identical to SEQ ID NO: 28
  Synthetic primer sequence

SEQ ID NO: 31 - DNA, 1992 bp, Candida albicans
atgaatgttttagtatattctggccccggcactacaactgagggcgtaaaacactgtttggagactttgagactccatttagggtcgtattatgctgttcttcctgttaacgagactgtgttattgaatgagccatggatgagaaaaacgtccttgttagttataccaggcggagcagatttaccatactgtaatgtacttgatggaaatggcacgagaaagatttccaagtatgtcaaacaaggtgggaagtttttagggctttgtgctggtgggtattttggatctgctaggtgtgagtttgaagttggcaatcctaccatggaggttactggacctagggaacttgggtttttcccaggaacggccaagggatgtgcatttaaaggtttcaagtacgaactgcgtactggtgcaagagccgtcaaattgctggtcaatacagctgcattacctggatgtgcaagtcacatttataattattacgatggcggggcggtgtttgccaatgctgaaaagtataaagatgttgaaatcttggcaaggtacgacgacaaaactgatattgtggatttagagaaggctgctgttgtatacagaaaggtcggtaagggtggtgttattttgagtggtactcatcctgaatttgcacctcacttgttgcatccacgagatgaagatggggcaggttattttatagtggttgacacacttcgagcatacgatcacaacaaaaaggtatttatgagagactgtttgaagaaattgggcttgcgagttgccgagagtgttgatacaacgattccaagagttacaccaatgtatgttgtatctccgttcaaggataaagttagggatgtatacctgattttaaccagcaagttaggtaagctgtttgaggatagtaatgatgctttttattttgctgacgaaacgcaggaaactagtgagtatgttggtagtgaagaagatccagtcaagtatatcaactttctcactctggccggtataccggatttgaaaatggttccctattttgacatccaaaagtattttgataatttgaggatgttgtcgggtggtgacatcaagtttgggagcattttaggttattccgaggtgattacatctacaaacacaattatggataagaatccgcaatggttagagcatcttcctaatgggtttaccattactgcaactacacaaatagcaggtagaggtcgcggtggcaatgtatgggttaacccccgcggtgtccttgcaacgtcagtgcttttcaaaataccaccttcaccctcgtcgtcgtcaacggtggttacattgcagtatttatgtggattggcattgattgagtcgattctagggtatggaagcaacgtttcagggcaaggcgttgggtatgaagatatgccgttgagattaaagtggcctaacgatatctttattatgaagccagagtatttcaagtcgttggacgataaaagtgatataagtgccactgttgatggtgacgatgagaagtttgtcaaggtttcaggtgcattgattaactcgcaatttatcaacaaaaccttttatttggtatggggaggtggagttaatgtatcaaatccagcaccaacaacgtcattaaacttggttttagaaaagttgaatgagatcagacgtggaaagggtttatcaccattaccaccatacgaaccagaaatattgcttgcaaagcttatgtttaccattgaccagttttattctgtatttgaaaagtcaggattgcaaccatttttgccgttatactataaacggtggttccataccaaccagaaagtggacgttgacaatggcagtgggaaacaaagaacatgtataatcaagggtattaccccagactatggattgcttattgctgaagatgtagagacgaaaaaagtgttgcatttacaaccagatggcaactcgtttgatatttttaaaggtttagtatataagaag

SEQ ID NO: 32 - DNA, 32 bp, Artificial Sequence
  Synthetic primer sequence
atctactcatgaatgttttagtatattctggc

SEQ ID NO: 33 - DNA, 39 bp, Artificial Sequence
  Synthetic primer sequence
gacggctctggcgccagtacgactttcgtacttgaaacc

SEQ ID NO: 34 - DNA, 44 bp, Artificial Sequence
  Synthetic primer sequence
cgtactggcgccagagccgtcaaattgagcgtcaatacagctgc

SEQ ID NO: 35 - DNA, 44 bp, Artificial Sequence
  Synthetic primer sequence
tccggtataccggcgctagtgagaaagttgatatacttgactgg

SEQ ID NO: 36 - DNA, 20 bp, Artificial Sequence
  Synthetic primer sequence
gtggatttagagaaggctgc

SEQ ID NO: 37 - DNA, 40 bp, Artificial Sequence
  Synthetic primer sequence
gtggttgacacacttcgagcatacgatcacaacaaaaagg

SEQ ID NO: 38 - DNA, 40 bp, Artificial Sequence
  Synthetic primer sequence
cctttttgttgtgatcgtatgctcgaagtgtgtcaaccac

SEQ ID NO: 39 - DNA, 22 bp, Artificial Sequence
  Synthetic primer sequence
actagtgagtatgttggtagtg

SEQ ID NO: 40 - DNA, 65 bp, Artificial Sequence
  Synthetic primer sequence
gagctcggtacctaatgatgatgatgatgatgatgatgatgaccggtcttcttatatactaaacc

SEQ ID NO: 41 - DNA, 2035 bp, Candida albicans
tcatgaatgttttagtatattctggccccggcaccacaactgagggcgtaaaacactgtttggagactttgagactccatttagggtcgtattatgctgttcttcctgttaacgagactgtgttattgaatgagccatggatgagaaaaacgtccttgttagttataccaggcggagcagatttaccatactgtaatgtacttgatggaaatggcacgagaaagatttccaagtatgtcaaacaaggtgggaagtttttagggctttgtgctggtgggtattttggatctgctaggtgtgagtttgaagttggcaatcctaccatggaggttactggacctagggaacttgggtttttcccaggaacggccaagggatgtgcatttaaaggtttcaagtacgaaagtcgtactggcgccagagccgtcaaattgagcgtcaatacagctgcattacctggatgtgcaagtcacatttataattattacgatggcggggcggtgtttgccaatgctgaaaagtataaagatgttgaaatcttggcaaggtacgacgacaaaactgatattgtggatttagagaaggctgctgttgtatacagaaaggtcggtaagggtggtgttattttgagtggtactcatcctgaatttgcacctcacttgttgcatccacgagatgaagatggggcaggttattttatagtggttgacacacttcgagcatacgatcacaacaaaaaggtatttatgagagactgtttgaagaaattgggcttgcgagttgccgagagtgttgatacaacgattccaagagttacaccaatgtatgttgtgtctccgttcaaggataaagttagggatgtgtacagcattttaaccagcaagttaggtaagctgtttgaggatagtaatgatgctttttattttgctgacgaaacgcaggaaactagtgagtatgttggtagtgaagaagatccagtcaagtatatcaactttctcactagcgccggtataccggatttgaaaatggttccctattttgacatccaaaagtattttgataatttgaggatgttgtcgggtggtgacatcaagtttgggagcattttaggttattccgaggtgattacatctacaaacacaattatggataagaatccgcaatggttagagcatcttcctaatgggtttaccattactgcaacaacacaaatagcaggtagaggtcgcggtggcaatgtatgggtcaacccccgcggtgtccttgcaacgtcagtgcttttcaaaataccaccttcaccctcgtcgtcgtcaacggtggttacattgcagtatttatgtggattggcattgattgagtcgattctagggtatggaagcaacgtttcagggcaaggcgttgggtatgaagatatgccgttgagattaaagtggcctaacgatatctttattatgaagccagagtatttcaagtcgttggacgataaaagtgatataagtgccactgttgatggtgacgatgagaagtttgtcaaggtttcaggtgcattgattaactcgcaatttatcaacaaaaccttttatttggtatggggaggtggagttaatgtatcaaatccagcaccaacaacgtcattaaacttggttttagaaaagttgaatgagatcagacgtggaaagggtttatcaccattaccaccatacgaaccagaaatattgcttgcaaagcttatgtttaccattgaccagttttattctgtatttgaaaagtcaggattgcaaccatttttaccgttatactataaacggtggttccataccaaccagaaagtggacgttgacaatggcagtgggaaacaaagaacatgtataatcaagggtattaccccagactacggattgcttattgctgaagatgtagagacgaaaaaagtgttgcatttacaaccagatggcaactcgtttgatatttttaaaggtttagtatataagaagaccggtcatcatcatcatcatcatcatcatcattaggtacc

SEQ ID NO: 42 - DNA, 2181 bp, Homo sapiens
atggaagatagactccacatggataatggactggtaccccaaaagattgtgtcggtgcacttgcaggactccactctgaaggaagttaaggatcaggtctcaaacaagcaagcccagatcctagagccgaagcctgaaccttctcttgagattaagcctgagcaggacggtatggagcatgttggcagagatgacccaaaggctcttggtgaagaacccaaacaaaggagaggcagtgcctctgggagtgagcctgctggggacagtgacaggggagggggccccgttgagcattatcacctccatctgtctagttgccacgagtgtctggaacttgagaacagcaccattgagtcagtcaagtttgcgtctgccgagaacattccagaccttccctacgattatagcagcagtttggagagtgttgctgatgagacctcccccgaaagagaagggaggagagtcaacctcacgggaaaggcacccaacatcctcctctatgtgggctccgactcccaggaagccctcggccggttccacgaggtccggtctgtgctggccgactgtgtggacattgacagttatattctctaccacctgctggaggacagtgctctcagagacccgtggacggacaactgtctgctgttggtcattgctaccagggagtccattcccgaagacctgtaccagaagttcatggcctatctttctcagggagggaaggtgttgggcctgtcttcatccttcacctttggtggctttcaggtgacaagcaagggtgcactgcacaagacagtccagaacttggttttctccaaggctgaccagagcgaggtgaagctcagcgtcttgagcagtggctgcaggtaccaggaaggccccgtccggctcagccccggcaggctccagggccacctggagaatgaggacaaggacaggatgattgtgcatgtgccttttggaactcgcgggggagaagctgttctttgccaggtgcacttagaactacctcccagctccaacatagtgcaaactccagaagattttaacttgctcaagtcaagcaattttagaagatacgaagtccttagagagattctgacaacccttggcctcagctgtgacatgaaacaagttcctgccttaactcctctttacttgctgtcagctgcggaggaaatcagggatcctcttatgcagtggcttgggaaacatgtggactccgagggagaaataaaatccggccagctctctcttagatttgtttcatcctacgtgtctgaagtagaaataaccccatcttgtatacctgtggtgaccaacatggaggccttctcatcagaacatttcaacttagagatctatcgccaaaatctgcagaccaagcagttggggaaagtaattttgtttgccgaagtgacccccacaacgatgcgtctcctggatgggctgatgtttcagacaccgcaggaaatgggcttaatagtgatcgcggcccggcagaccgagggcaaaggacggggagggaatgtgtggctgagccctgtgggatgtgctctttctactctgctcatctccattccactgagatcccagctgggacagaggatcccgtttgtccagcatctgatgtccgtggctgtcgtggaagcagtgaggtccattcccgagtatcaggatatcaacttacgagtgaagtggcccaacgatatttattacagtgacctcatgaagatcggcggagttctggttaactcaacactcatgggagaaacattttatatacttattggctgtggatttaatgtgactaacagtaaccctaccatctgcatcaacgacctcatcacagaatacaataaacaacacaaggcagaactgaagcccttaagagccgattatctcatcgccagagtcgtgactgtgctggagaaactgatcaaagagtttcaggacaaagggcccaacagcgtccttcccctttattaccgatactgggtccacagtggtcagcaagtccatctgggcagcgcagagggaccaaaggtgtccatcgttggcctggacgattctggcttcctccaggttcaccaggagggcggcgaggttgtgactgtgcacccggacggcaactccttcgacatgctgagaaacctcatcctccccaaacggcggtaa

SEQ ID NO: 43 - DNA, 35 bp, Artificial Sequence
  Synthetic primer sequence
atataaccatggaagatagactccacatggataat

SEQ ID NO: 44 - DNA, 18 bp, Artificial Sequence
  Synthetic primer sequence
ggagacgcatcgttgtgg

SEQ ID NO: 45 - DNA, 20 bp, Artificial Sequence
  Synthetic primer sequence
cttgtatacctgtggtgacc

SEQ ID NO: 46 - DNA, 64 bp, Artificial Sequence
  Synthetic primer sequence
ataatccctactcgagctaatgatgatgatgatgatgccgccgtttggggaggatgaggtttct

SEQ ID NO: 47 - DNA, 29 bp, Artificial Sequence
  Synthetic primer sequence
aaggagagccatggcctctgggagtgagc

SEQ ID NO: 48 - DNA, 23 bp, Artificial Sequence
  Synthetic primer sequence
tcctgtccttgtcctcattctcc

SEQ ID NO: 49 - DNA, 1973 bp, Homo sapiens
ccatggcctctgggagtgagcctgctggggacagtgacaggggagggggccccgttgagcattatcacctccatctgtctagttgccacgagtgtctggaacttgagaacagcaccattgagtcagtcaagtttgcgtctgccgagaacattccagaccttccctacgattatagcagcagtttggagagtgttgctgatgagacctcccccgaaagagaagggaggagagtcaacctcacgggaaaggcacccaacatcctcctctatgtgggctccgactcccaggaagccctcggccggttccacgaggtccggtctgtgctggccgactgtgtggacattgacagttatattctctaccacctgctggaggacagtgctctcagagacccgtggacggacaactgtctgctgttggtcattgctaccagggagtccattcccgaagacctgtaccagaagttcatggcctatctttctcagggagggaaggtgttgggcctgtcttcatccttcacctttggtggctttcaggtgacaagcaagggtgcactgcacaagacagtccagaacttggttttctccaaggctgaccagagcgaggtgaagctcagcgtcttgagcagtggctgcaggtaccaggaaggccccgtccggctcagccccggcaggctccagggccacctggagaatgaggacaaggacaggatgattgtgcatgtgccttttggaactcgcgggggagaagctgttctttgccaggtgcacttagaactacctcccagctccaacatagtgcaaactccagaagattttaacttgctcaagtcaagcaattttagaagatacgaagtccttagagagattctgacaacccttggcctcagctgtgacatgaaacaagttcctgccttaactcctctttacttgctgtcagctgcggaggaaatcagggatcctcttatgcagtggcttgggaaacatgtggactccgagggagaaataaaatccggccagctctctcttagatttgtttcatcctacgtgtctgaagtagaaataaccccatcttgtatacctgtggtgaccaacatggaggccttctcatcagaacatttcaacttagagatctatcgccaaaatctgcagaccaagcagttggggaaagtaattttgtttgccgaagtgacccccacaacgatgcgtctcctggatgggctgatgtttcagacaccgcaggaaatgggcttaatagtgatcgcggcccggcagaccgagggcaaaggacggggagggaatgtgtggctgagccctgtgggatgtgctctttctactctgctcatctccattccactgagatcccagctgggacagaggatcccgtttgtccagcatctgatgtccgtggctgtcgtggaagcagtgaggtccattcccgagtatcaggatatcaacttacgagtgaagtggcccaacgatatttattacagtgacctcatgaagatcggcggagttctggttaactcaacactcatgggagaaacattttatatacttattggctgtggatttaatgtgactaacagtaaccctaccatctgcatcaacgacctcatcacagaatacaataaacaacacaaggcagaactgaagcccttaagagccgattatctcatcgccagagtcgtgactgtgctggagaaactgatcaaagagtttcaggacaaagggcccaacagcgtccttcccctttattaccgatactgggtccacagtggtcagcaagtccatctgggcagcgcagagggaccaaaggtgtccatcgttggcctggacgattctggcttcctccaggttcaccaggagggcggcgaggttgtgactgtgcacccggacggcaactccttcgacatgctgagaaacctcatcctccccaaacggcggcatcatcatcatcatcattagctcgag

SEQ ID NO: 50 - DNA, 966 bp, Escherichia coli
atgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaataa

SEQ ID NO: 51 - protein, 86 aa, Escherichia coli; identical to SEQ ID NO: 5

SEQ ID NO: 52 - DNA, 32 bp, Artificial Sequence
  Synthetic primer sequence
atctacggatccatggaagcgccagcagcagc

SEQ ID NO: 53 - DNA, 34 bp, Artificial Sequence
  Synthetic primer sequence
atctacgaattcatcactcgatgacgaccagcgg

SEQ ID NO: 54 - DNA, 1104 bp, Arabidopsis thaliana
atggaagcagttcgttcaacaacaaccttatctaattttcatctactgaatattttagtcctgcgttctctcaagcctctgcatcgtctatctttctctttctctgcctcagcaatggagagtgatgcatcttgtagcttagttttgtgtgggaaatcctctgtggaaactgaggtagcaaagggtttgaagaacaagaactctcttaaactcccagataataccaaagtttcactcattttagaatctgaggctaagaatttggttaaagatgatgataattccttcaatctttcactctttatgaactcgattataactcatcggtttggacggtttctcatttggtctccgcgtttgtcttccactcacgatgttgtttctcataacttctctgagcttccagttggttcagtttgtgtcactgatatccagtttaagggtcgaggcagaacaaagaatgtatgggaatctccaaagggttgtcttatgtattcttttactctagaaatggaagatggtcgagtcgtgcctttgatacagtatgtggtatctcttgctgtaactgaggcagtgaaagatgtttgtgacaagaagggtttgccgtacattgatgttaaaataaagtggccaaatgatctgtacgtgaatggtcttaaggttggaggcattttgtgtacctctacgtatagatcaaagaagttcaatgtcagtgttggtgtggggttgaatgtggacaacgggcaaccgaccacgtgcttaaatgcagtactaaaaggcatggctcctgaatcaaacctacttaaaagagaggaaatccttggtgccttctttcataaatttgaaaaattcttcgatctattcatggaccaaggttttaaatctctcgaagagctttactacaggacatggctacacagcgagcaaagagtgattgttgaagacaaagtagaggaccaagttgttcaaaatgttgtgactatccagggtttgacttcttctggatatttgctggctgttggagatgacaatcaaatgtatgagcttcaccctgatggcaatagttttgactttttcaaaggtctggttcgaagaaaaatatga

SEQ ID NO: 55 - DNA, 35 bp, Artificial Sequence
  Synthetic primer sequence
atctatccatggaagcagttcgttcaacaacaacc

SEQ ID NO: 56 - DNA, 68 bp, Artificial Sequence
  Synthetic primer sequence
atccatggatcctaatgatgatgatgatgatgatgatgatgaccggttatttttcttcgaaccagacc

SEQ ID NO: 57 - DNA, 1144 bp, Arabidopsis thaliana
ccatggaagcagttcgttcaacaacaaccttatctaattttcatctactgaatattttagtcctgcgttctctcaagcctctgcatcgtctatctttctctttctctgcctcagcaatggagagtgatgcatcttgtagcttagttttgtgtgggaaatcctctgtggaaactgaggtagcaaagggtttgaagaacaagaactctcttaaactcccagataataccaaagtttcactcattttagaatctgaggctaagaatttggttaaagatgatgataattccttcaatctttcactctttatgaactcgattataactcatcggtttggacggtttctcatttggtctccgcgtttgtcttccactcacgatgttgtttctcataacttctctgagcttccagttggttcagtttgtgtcactgatatccagtttaagggtcgaggcagaacaaagaatgtatgggaatctccaaagggttgtcttatgtattcttttactctagaaatggaagatggtcgagtcgtgcctttgatacagtatgtggtatctcttgctgtaactgaggcagtgaaagatgtttgtgacaagaagggtttgccgtacattgatgttaaaataaagtggccaaatgatctgtacgtgaatggtcttaaggttggaggcattttgtgtacctctacgtatagatcaaagaagttcaatgtcagtgttggtgtggggttgaatgtggacaacgggcaaccgaccacgtgcttaaatgcagtactaaaaggcatggctcctgaatcaaacctacttaaaagagaggaaatccttggtgccttctttcataaatttgaaaaattcttcgatctattcatggaccaaggttttaaatctctcgaagagctttactacaggacatggctacacagcgagcaaagagtgattgttgaagacaaagtagaggaccaagttgttcaaaatgttgtgactatccagggtttgacttcttctggatatttgctggctgttggagatgacaatcaaatgtatgagcttcaccctgatggcaatagttttgactttttcaaaggtctggttcgaagaaaaataaccggtcatcatcatcatcatcatcatcatcattaggatcc

SEQ ID NO: 58 - protein, 93 aa, Arabidopsis thaliana
PSPPTPAKSSLPTVKSPMAGTFYRSPAPGEPPFIKVGDKVQKGQVLCIVEAMKLMNEIESDHTGTVVDIVAEDGKPVSLDTPLFVVQPVESAP

SEQ ID NO: 59 - DNA, 32 bp, Artificial Sequence
  Synthetic primer sequence
atctacggatccccaccatccccacctactcc

SEQ ID NO: 60 - DNA, 33 bp, Artificial Sequence
  Synthetic primer sequence
atctacgaattcatcatggtgccgattctacgg

SEQ ID NO: 61 - DNA, 3126 bp, Drosophila melanogaster
atgttgaccctgtattacgtgagtgccaccgtcctgcagtcctggcgcattcaaaaggcgtgctccaagatcgccgagcacctcgcgcagccctcgagcatcgccttctataccctccaatcgggcagcgatgacggattcgatcccgccctggccagctcggagttgtgctgcaatcggaacgcggccaaggtcacggacatcctgtggctgcacgccaaccagcggggttgctgcctgcgtccgctgcaaacgctacacatcacgccttggatcagctttccgccggcgcccagtctgcttcccttctcctatgccgccgacacgttaacgccggcgtccacgcccacggaggcggacgtcccgcgccaacaacgggtttcgctctctgcccaaggggaggagcggatgcagctgctccttgaggccgacatagagccactacagcggcccagctccgaggacacatcagcagtacggctggaagattacggtaagctaattgcttggaaaatagattcccacctggcggtgctcatcgaaacagacgtcgagcacttcacgaaacttttgataacgactttcttgagaaataatttatgcattaacgatcagctgccacttttgcggattgagtcggtgcaacgggaaggggatccgcagccctttgagcttctggccaagcatctgaagcggcaatcccgcataagcgtcggtctggacaaggatggatggaagaagcacatggaggacttgcgtgccgtaggtgtgctggcgcaccaggccaccgagttcgagtaccagaggaatcgctctgagggaagaacgaaatcggatcccaccacacatgatcaacgcccgacttcggagcttttggccaaggctgtggccgtggtggagcccaccaacaaggcatatctgtctccggcaaggacgacagaagcctcgctcaaagccgaggccaagggaacgtcaacaaaaccttttccgactaaaagcgatgccaaacccgccactcttgccaagagcgagggcacgccagccacaagcaaagaggactccaagttaacgccaactaagggtgcattaaagacaagcgaactagagaaactagcggctgtccaggcagcgcagcagcagaagaaagaggcagtccaggtctctcccgtcaaggctgctttcttagctaaaccacctatgctaagtaaacatgatgagcctgataaggcatcagatcagccaacgaagcccaggaaatcctttgaggcggtaaagccgttaaattctcctccgtcatccagagctgctccctcgcgtcctgtgctgcaacgcaacaaggatagcctgcaggacgcaaaaccattgaatgttttggtctactcagacagcgccagtgcgcgggaatccacattggccactctgcagcagctgctggagcgcaatgtgtacacgatctatcctctgatgcctcagcaagcggctcaaaaatactggacggaacagactgcgttgctggtcgtctgcggatcggtggcacacggaattgggcagatattggtggactactttctgcagggcggcaaggtgttaagcctgtgctcggatatactgcactttgtactccccaactatcgcacagcggaggttcgagagcacgaattggttcagttctcctatgacaagtggcagcgggtcaagatgatgcaccacatcttttgctaccaaccgtcgccggtgaagaagcacttctccactgacagcgaggagtccaccaagtcgcactctcgcaaaccgtccatggaactgaaggatctggccggtcacagccacaatttggatgtccatgtgctgggaaccgaggaaacctggaatacgcccagcttgatgttggccaaaagcctgcagagcggcggaaaggccgtcttctcccaagtgcacttggaaatgaatcccagcgaatttgaatccgatgagactaagtactccattctaaagcaaaacgagcgaactcgcctcgagatatttgcggatctcctgggaaagtacttggatgtgcaggtccgcggtggagacggtgtcgaccagccgcagcctggagttgtctacaagcacgcatatttcctaggtcggcacgaggccaagtttgagcttctggagaagctacgtctgcgctgcagcggatccgacaacgtgatagcaacgccgaacctgacgatgaagttctgtggcaaggacgataagcctccagtggccaacaacaatgtcctgcccatactcatacattcctgtccggatgacttctccaccgtggattacttcgataatcttaagacggaacacatcggacgactggtcatctatgcaccggtggtcagcagctccatgcatctcataaacaacctggagctcatccacggcctggctgtgctccccgtgcagcagacgtccggagtgggtcgcagaaataatcaatggcttagtccgccgggctgtgccatgttctcgctgcagctgcacctgaccatggacagcgctttgagctctcggctgcctctgttgcagcaccttgttggaacggcgattgttaattcgctccgcagtcatgaagagtacggggtgctggatatttccattaagtggccgaatgacatttatgcaaatggaaatcagaaaattgggggcctcgtcattaataccacacttcagggatcccaagccatcgtcaatatcggaagtggaattaatttgaacaattcaagaccaacagtttgcatcaatgacttgataagagaatataatacccgtgtcccgaacaacaagttacctatacttaagtacgagctacttattgccatgatattcaatgaaatcgaaagacttttgggagaagtacaaaacggagactttgatagtttttatgcattatactattcgctatggttacacagcgggcaatctgttaagatttgcctacaaaaagatcaggaaaaagaggccgaaattgtgggaatcgatgactttggatttttggaggtgaaattaccaactggtactatagaaattgtgcagcccgatggaaacagctttgatatgttgaagggattgattatacccaagtatcaatag

SEQ ID NO: 62 - DNA, 29 bp, Artificial Sequence
  Synthetic primer sequence
gaattcatgttgaccctgtattacgtgag

SEQ ID NO: 63 - DNA, 27 bp, Artificial Sequence
  Synthetic primer sequence
aagcttcatttcactattgatacttgg

SEQ ID NO: 64 - protein, 30 aa, Artificial Sequence
  Synthetic peptide - six histidine extension
MSYYHHHHHHDYDIPTTENLYFQGAMDPEF

SEQ ID NO: 65 - DNA, 3227 bp, Drosophila melanogaster
atgtcgtactaccatcaccatcaccatcacgattacgatatcccaacgaccgaaaacctgtattttcagggcgccatggatccggaattcatgttgaccctgtattacgtgagtgccaccgtcctgcagtcctggcgcattcaaaaggcgtgctccaagatcgccgagcacctcgcgcagccctcgagcatcgccttctataccctccaatcgggcagcgatgacggattcgatcccgccctggccagctcggagttgtgctgcaatcggaacgcggccaaggtcacggacatcctgtggctgcacgccaaccagcggggttgctgcctgcgtccgctgcaaacgctacacatcacgccttggatcagctttccgccggcgcccagtctgcttcccttctcctatgccgccgacacgttaacgccggcgtccacgcccacggaggcggacgtcccgcgccaacaacgggtttcgctctctgcccaaggggaggagcggatgcagctgctccttgaggccgacatagagccactacagcggcccagctccgaggacacatcagcagtacggctggaagattacggtaagctaattgcttggaaaatagattcccacctggcggtgctcatcgaaacagacgtcgagcacttcacgaaacttttgataacgactttcttgagaaataatttatgcattaacgatcagctgccacttttgcggattgagtcggtgcaacgggaaggggatccgcagccctttgagcttctggccaagcatctgaagcggcaatcccgcataagcgtcggtctggacaaggatggatggaagaagcacatggaggacttgcgtgccgtaggtgtgctggcgcaccaggccaccgagttcgagtaccagaggaatcgctctgagggaagaacgaaatcggatcccaccacacatgatcaacgcccgacttcggagcttttggccaaggctgtggccgtggtggagcccaccaacaaggcatatctgtctccggcaaggacgacagaagcctcgctcaaagccgaggccaagggaacgtcaacaaaaccttttccgactaaaagcgatgccaaacccgccactcttgccaagagcgagggcacgccagccacaagcaaagaggactccaagttaacgccaactaagggtgcattaaagacaagcgaactagagaaactagcggctgtccaggcagcgcagcagcagaagaaagaggcagtccaggtctctcccgtcaaggctgctttcttagctaaaccacctatgctaagtaaacatgatgagcctgataaggcatcagatcagccaacgaagcccaggaaatcctttgaggcggtaaagccgttaaattctcctccgtcatccagagctgctccctcgcgtcctgtgctgcaacgcaacaaggatagcctgcaggacgcaaaaccattgaatgttttggtctactcagacagcgccagtgcgcgggaatccacattggccactctgcagcagctgctggagcgcaatgtgtacacgatctatcctctgatgcctcagcaagcggctcaaaaatactggacggaacagactgcgttgctggtcgtctgcggatcggtggcacacggaattgggcagatattggtggactactttctgcagggcggcaaggtgttaagcctgtgctcggatatactgcactttgtactccccaactatcgcacagcggaggttcgagagcacgaattggttcagttctcctatgacaagtggcagcgggtcaagatgatgcaccacatcttttgctaccaaccgtcgccggtgaagaagcacttctccactgacagcgaggagtccaccaagtcgcactctcgcaaaccgtccatggaactgaaggatctggccggtcacagccacaatttggatgtccatgtgctgggaaccgaggaaacctggaatacgcccagcttgatgttggccaaaagcctgcagagcggcggaaaggccgtcttctcccaagtgcacttggaaatgaatcccagcgaatttgaatccgatgagactaagtactccattctaaagcaaaacgagcgaactcgcctcgagatatttgcggatctcctgggaaagtacttggatgtgcaggtccgcggtggagacggtgtcgaccagccgcagcctggagttgtctacaagcacgcatatttcctaggtcggcacgaggccaagtttgagcttctggagaagctacgtctgcgctgcagcggatccgacaacgtgatagcaacgccgaacctgacgatgaagttctgtggcaaggacgataagcctccagtggccaacaacaatgtcctgcccatactcatacattcctgtccggatgacttctccaccgtggattacttcgataatcttaagacggaacacatcggacgactggtcatctatgcaccggtggtcagcagctccatgcatctcataaacaacctggagctcatccacggcctggctgtgctccccgtgcagcagacgtccggagtgggtcgcagaaataatcaatggcttagtccgccgggctgtgccatgttctcgctgcagctgcacctgaccatggacagcgctttgagctctcggctgcctctgttgcagcaccttgttggaacggcgattgttaattcgctccgcagtcatgaagagtacggggtgctggatatttccattaagtggccgaatgacatttatgcaaatggaaatcagaaaattgggggcctcgtcattaataccacacttcagggatcccaagccatcgtcaatatcggaagtggaattaatttgaacaattcaagaccaacagtttgcatcaatgacttgataagagaatataatacccgtgtcccgaacaacaagttacctatacttaagtacgagctacttattgccatgatattcaatgaaatcgaaagacttttgggagaagtacaaaacggagactttgatagtttttatgcattatactattcgctatggttacacagcgggcaatctgttaagatttgcctacaaaaagatcaggaaaaagaggccgaaattgtgggaatcgatgactttggatttttggaggtgaaattaccaactggtactatagaaattgtgcagcccgatggaaacagctttgatatgttgaagggattgattatacccaagtatcaatagaaatgaagctt

SEQ ID NO: 66 - protein, 124 aa, Drosophila melanogaster; identical to SEQ ID NO: 4

SEQ ID NO: 67 - DNA, 35 bp, Artificial Sequence
  Synthetic primer sequence
atctacggatccggtaagacgctgagcgtgaaagc

SEQ ID NO: 68 - DNA, 37 bp, Artificial Sequence
  Synthetic primer sequence
atctacgaattcctattcgataatcataatgaggtcc

(19) United States
(12) Patent Application Publication
(10) Pub. No.: US 2009/0118372 A1
(43) Pub. Date: May 7, 2009

(54) Inhibitors Of Biotin Protein Ligase

(75) Inventors: Steven William Polyak, South Australia (AU); Grant William Booker, South Australia (AU); Cvetan Stojkoski, South Australia (AU); John Campbell Wallace, North Adelaide (AU)

Correspondence Address: BIRCH STEWART KOLASCH & BIRCH, PO BOX 747, FALLS CHURCH, VA, 22040-0747, US

(73) Assignee: Adelaide Research & Innovation Pty Ltd., Adelaide (AU)

(21) Appl. No.: 11/791,234

(22) PCT Filed: Nov. 23, 2005
(86) PCT No.: PCT/AU2005/001768

§ 371 Date: Mar. 25, 2008

(30) Foreign Application Priority Data
Nov. 23, 2004 (AU) 2004906688

(51) Int. Cl. A61K 31/194; C12Q 1/00; C12N 9/00; C12Q 1/02; C07C 59/48

(52) U.S. Cl. 514/570; 435/4; 435/183; 435/29; 562/465

(57) ABSTRACT

The present invention relates to a method for identifying an inhibitor of a biotin protein ligase. The method include the steps of (a) providing a substrate, wherein the substrate may be biotinylated; (b) contacting the substrate with biotin and a biotin protein ligase in the presence of a test compound; (c) determining the extent of biotinylation of the substrate by the biotin protein ligase in the presence of the test compound; and (d) identifying the test compound as an inhibitor of the biotin protein ligase by a reduction in the biotinylation of the substrate in the presence of the test compound as compared to the extent of biotinylation of the substrate in the absence of the test compound.

This application claims priority from Australian Provisional Patent Application No. 2004906688 filed on 23 Nov. 2004, the contents of which are to be taken as incorporated herein by this reference.

### FIELD OF THE INVENTION

The present invention relates to methods for identifying inhibitors of biotin protein ligases, and in particular, to methods for the identification of inhibitors of biotin protein ligases of pathogenic organisms. The present invention also relates to methods for identifying differential inhibitors of biotin protein ligases.

The present invention also relates to inhibitors of biotin protein ligases identified by the methods of the present invention, biotin protein ligases as targets for identifying inhibitors of biotinylation, and methods and compositions for inhibiting biotinylation using the inhibitors.

### BACKGROUND OF THE INVENTION

There is a continuing need for the development of new agents that have the ability to inhibit the growth of pathogenic organisms, and in particular, the development of new agents that have the ability to inhibit the growth of bacterial and fungal pathogens. Infection by bacterial and fungal pathogens represents one of the most significant causes of disease and economic loss.

The treatment of bacterial infections has traditionally relied on the use of antibiotics. However, the treatment of many bacterial infections is becoming increasingly difficult because of resistance to one or more known antibiotics developing in the bacteria. Indeed, in some cases bacteria have become resistant to all known antibiotics, rendering the host organism vulnerable to the effects of infection by the bacteria.

The effective treatment of fungal and parasitic infections also remains problematic. For example, strains of the yeast Candida are among the most virulent pathogenic fungal organisms of humans. While there are several topical agents for treatment of candidiases, treatment of systemic infection by Candida is much more difficult Systemic fungal infections are common complications in immune-compromised patients, and as such may be life-threatening. The available drugs for treating systemic fungal infections are often highly toxic to the host.

Plants are also infected by a wide range of pathogens, including bacterial, fungal nematode and insect pathogens. The infection of plants by such pathogens causes extensive losses to agricultural products and also affects the storage and manufacture of agricultural products.

Accordingly, there is a need for the identification of new agents that may prevent or ameliorate infection by pathogenic organisms. In addition, it is desirable that such agents are able not only to kill or significantly inhibit the growth of the pathogenic organism, but they should also not be highly toxic to the host organism.

Biotinylation is a process that is ubiquitous to all organisms. In this process biotin, otherwise known as vitamin H, is covalently attached at the active site of the class of metabolic enzymes known as biotin carboxylases, biotin decarboxylases and biotin transcarboxylases. These are key enzymes involved in gluconeogenesis, lipogenesis, amino acid metabolism and energy transduction. Biotin must be covalently attached to these enzymes for the enzymes to function. For example, biotin must be covalently attached to pyruvate carboxylase so that the enzyme may catalyse the generation of oxaloacetate, a precursor for the synthesis of glucose and fat as well as some amino acids and neurotransmitters.

The enzyme responsible for the covalent attachment of biotin to its cognate proteins is biotin protein ligase (BPL). Biotin is attached post-translationally by BPL in a reaction of stringent specificity via an amide linkage to a specific lysine residue in a two-step reaction as shown in FIG. 1.

Despite the fact that biotin-dependent enzymes are present in all organisms, biotinylation is still a rare modification in the cell, with only between one and five distinct protein species actually being biotinylated. The functional interaction between BPL and its protein substrates shows a very high degree of conservation throughout evolution, because biotinylation will occur when the two proteins are from widely-divergent biological sources.

As biotinylation is a process that is ubiquitous in all organisms, it represents a potential target for the development of new compounds that may inhibit the growth of pathogenic organisms. The present invention relates to methods for the identification of inhibitors of biotin protein ligase.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention provides a method for identifying an inhibitor of a biotin protein ligase, the method including the steps of:
(a) providing a substrate, wherein the substrate may be biotinylated;
(b) contacting the substrate with biotin and a biotin protein ligase in the presence of a test compound;
(c) determining the extent of biotinylation of the substrate by the biotin protein ligase in the presence of the test compound; and
(d) identifying the test compound as an inhibitor of the biotin protein ligase by a reduction in the biotinylation of the substrate in the presence of the test compound as compared to the extent of biotinylation of the substrate in the absence of the test compound.

The present invention also provides an anti-pathogenic agent, wherein the agent inhibits biotinylation of a substrate by a biotin protein ligase of a pathogen.

The present invention also provides a method for identifying an agent that inhibits growth and/or survival of a pathogenic organism, the method including the step of identifying an agent that inhibits a biotin protein ligase.

The present invention also provides the use of a biotin protein ligase as a target for identifying an agent that inhibits growth and/or survival of an organism.

The present invention also provides an isolated biotin protein ligase suitable as a target for identifying an inhibitor of biotinylation.

The present invention also provides an isolated biotin protein ligase suitable for use as a target for identifying an agent that inhibits growth and/or survival of an organism.

The present invention also provides a method for identifying an inhibitor of biotinylation in a biological system, the method including the steps of:
(a) identifying a test compound as an inhibitor of a biotin protein ligase;
(b) determining the ability of the test compound to inhibit biotinylation in a biological system; and
(c) identifying the test compound as an inhibitor of biotinylation in the biological system.

The present invention also provides a method for identifying a compound that differentially inhibits a first biotin protein ligase as compared to inhibition of a second biotin protein ligase, the method including the steps of:
(a) identifying a test compound that inhibits a first biotin protein ligase;
(b) determining the ability of the test compound to inhibit a second biotin protein ligase; and
(c) identifying the test compound as a compound that differentially inhibits the first biotin protein ligase as compared to the inhibition of the second biotin protein ligase.

The present invention also provides a method for identifying a compound that differentially inhibits biotinylation in a first biological system as compared to inhibition of biotinylation in a second biological system, the method including the steps of:
(a) identifying a test compound that inhibits biotinylation in a first biological system;
(b) determining the ability of the test compound to inhibit biotinylation in a second biological system; and
(c) identifying the test compound as a compound that differentially inhibits biotinylation in the first biological system as compared to the inhibition of biotinylation in the second biological system.

The present invention also provides a method for preventing and/or treating an infection by a pathogenic organism of a subject, the method including the step of administering to the subject an effective amount of an agent that inhibits a biotin protein ligase of the pathogenic organism.

The present invention also provides a composition for preventing and/or treating an infection of a host by a pathogenic organism, the composition including a compound with the formula:

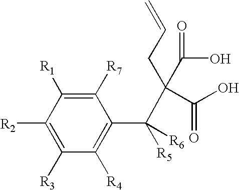

or a salt or ester thereof, wherein R₁ to R₇ are each independently selected from the group consisting of: H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, arylalkyloxy, phenoxy, benzyloxy, heteroaryloxy, amino, alkylamino, aminoalkyl, acylamino, arylamino, sulfonylamino, sulfinylamino, COOH, COR₈, COOR₈, CONHR₈, NHCOR, NHCOOR₈, NHCONHR₈, alkoxycarbonyl, alkylaminocarbonyl, sulfonyl, alkylsulfonyl, alkylsulfinyl, arylsulfonyl, arylsulfinyl, aminosulfonyl, SR₈, R₉S(O)R₁₀—, R₉S(O)₂R₁₀—, R₉C(O)N(R₁₀)R₁₁—, R₉SO₂N(R₁₀)R₁₁—, R₉N(R₁₀)C(O)R₁₁—, R₉N(R₁₀)SO₂R₁₁—, R₉N(R₁₀)C(O)N(R₁₀)R₁₁— and acyl, each of which may be optionally substituted; each R₈, R₉, R₁₀ and R₁₁ is independently selected from the group consisting of a bond, H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

The present invention also provides the use of a compound with the formula:

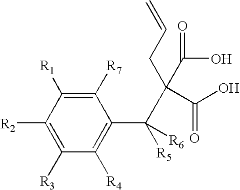

or a salt or ester thereof, in the preparation of a medicament for preventing and/or treating an infection of a host by a pathogenic organism, wherein R₁ to R₇ are each independently selected from the group consisting of: H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, arylalkyloxy, phenoxy, benzyloxy, heteroaryloxy, amino, alkylamino, aminoalkyl, acylamino, arylamino, sulfonylamino, sulfinylamino, COOH, COR₈, COOR₈, CONHR₈, NHCOR, NHCOOR₈, NHCONHR₈, alkoxycarbonyl, alkylaminocarbonyl, sulfonyl, alkylsulfonyl, alkylsulfinyl, arylsulfonyl, arylsulfinyl, aminosulfonyl, SR₈, R₉S(O)R₁₀—, R₉S(O)₂R₁₀—, R₉C(O)N(R₁₀)R₁₁—, R₉SO₂N(R₁₀)R₁₁—, R₉N(R₁₀)C(O)R₁₁—, R₉N(R₁₀)SO₂R₁₁—, R₉N(R₁₀)C(O)N(R₁₀)R₁₁— and acyl, each of which may be optionally substituted; each R₈, R₉, R₁₀ and R₁₁ is independently selected from the group consisting of a bond, H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

The present invention arises out of studies into the biotinylation of protein fragments by biotin protein ligases. In particular, it has been found that certain compounds are capable of inhibiting the biotinylation of protein fragments by biotin protein ligases, demonstrating that inhibitors of biotin protein ligases may be identified. Compounds so identified are candidate drugs for inhibiting the growth and/or survival of organisms.

In addition, it has also been found that some compounds are capable of differentially inhibiting biotinylation of a substrate by biotin protein ligases of different species. Given the high degree of conservation between biotin protein ligases of different species, the ability to identify compounds that differentially inhibit biotin protein ligases of different organisms was contrary to expectation.

In the case where the compounds differentially inhibit the biotin protein ligase of a pathogenic organism as compared to the host organism, these compounds are candidate drugs for selectively targeting the pathogen.

Various terms that will be used throughout the specification have meanings that will be well understood by a skilled addressee. However, for ease of reference, some of these terms will now be defined.

The term “biotin protein ligase” as used throughout the specification is to be understood to mean any protein (or a functional fragment thereof) that has the capacity to enzymatically attach a free biotin group covalently to a substrate in a reaction catalysed by the hydrolysis of a nucleoside triphosphate. The biotin protein ligase may be a naturally occurring form of the protein or a fragment thereof, a functional variant of the protein or fragment, a synthetic form of the protein, or an analogue of the protein. For example, the biotin protein ligase may be a protein or fragment thereof isolated from a cell that has the capacity to enzymatically attach a biotin group covalently to a substrate, or it may be any polypeptide synthesised in vitro (for example by chemical synthesis or by in vitro translation) that has the capacity to enzymatically attach a biotin group covalently to a substrate.

Methods for identifying biotin protein ligases are known in the art. For example, biotin protein ligases may be identified using the BLAST algorithm, which determines the extent of homology between two nucleotide sequences (blastn) or the extent of homology between two amino acid sequences (blastp). BLAST identifies local alignments between the sequences in the database and predicts the probability of the local alignment occurring by chance. The BLAST algorithm is as described in Altschul et al. (1990) J. Mol. Biol. 215:403-410.

The term “substrate” as used throughout the specification is to be understood to mean any molecule that has the capacity to have a biotin group attached covalently to the molecule by the action of a biotin protein ligase. Examples of suitable substrates include proteins or fragments thereof that have the capacity for a biotin group to be attached to them, polypeptides synthesized in vitro that have the capacity for a biotin group to be attached to them, or small molecules that have the capacity for a biotin group to be attached to them, such as hydroxylamine.

The term “biotinylation” as used throughout the specification is to be understood to mean the covalent attachment of a biotin group to one or more molecules. The biotinylation reaction may occur, for example, in vivo, in one or more isolated cells, or in a cell free system in vitro.

The term “biological system” as used throughout the specification is to be understood to mean any single or multi-cellular system in which biotinylation occurs. For example, the biological system may be one or more isolated cells, an unicellular organism, the part or whole of a tissue or organ, or an entire multi-cellular organism, such as a human, animal or plant.

The term “variant” as used throughout the specification is to be understood to mean an amino acid sequence of a polypeptide or protein that is altered by one or more amino acids. The variant may have “conservative” changes, wherein a substituted amino acid has similar structural or chemical properties to the replaced amino acid (e.g., replacement of leucine with isoleucine). A variant may also have “non-conservative” changes (e.g., replacement of a glycine with a tryptophan) or a deletion and/or insertion of one or more amino acids. The term also includes within its scope any insertions/deletions of amino acids to a particular polypeptide or protein. A “functional variant” will be understood to mean a variant that retains the functional capacity of a reference protein or polypeptide.

The term “nucleic acid” as used throughout the specification is to be understood to mean to any oligonucleotide or polynucleotide. The nucleic acid may be DNA or RNA and may be single stranded or double stranded. The nucleic acid may be any type of nucleic acid, including a nucleic acid of genomic origin, cDNA origin (ie derived from a mRNA), derived from a virus, or of synthetic origin.

In this regard, an oligonucleotide or polynucleotide may be modified at the base moiety, sugar moiety, or phosphate backbone, and may include other appending groups to facilitate the function of the nucleic acid. The oligonucleotide or polynucleotide may be modified at any position on its structure with constituents generally known in the art. For example, an oligonucleotide may include at least one modified base moiety which is selected from the group including 5-fluorouracil, 5-bromouracil, 5-chlorouracil, 5-iodouracil, hypoxanthine, xanthine, 4-acetylcytosine, 5-(carboxylhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil5-oxyacetic acid methylester, uracil-5-oxyacetic acid (v), 5-methyl-2-thiouracil, 3-(3-amino-3-N-2-carboxypropyl) uracil, (acp3) w, and 2,6-diaminopurine.

The oligonucleotide or polynucleotide may also include at least one modified sugar moiety selected from the group including, but not limited to, arabinose, 2-fluoroarabinose, xylulose, and hexose. In addition, the oligonucleotide or polynucleotide may include at least one modified phosphate backbone, such as a phosphorothioate, a phosphorodithioate, a phosphoramidothioate, a phosphoramidate, a phosphordiamidate, a methylphosphonate, an alkyl phosphotriester, and a formacetal or any analogue thereof.

The term “subject” as used throughout the specification is to be understood to mean any multicellular organism, including a human, plant or an animal subject.

For example, in the case where the subject is a human or animal, the subject organism may be a mammal, a primate, a livestock animal (eg. a horse, a cow, a sheep, a pig, or a goat), a companion animal (eg. a dog, a cat), a laboratory test animal (eg. a mouse, a rat, a guinea pig, a bird), an animal of veterinary significance, or an animal of economic significance.

In the case where the subject is a plant, the plant may be for example a commercial crop species (eg barley, oat, millet, alfalfa), a leguminous plant (eg soybean, alfalfa, and pea), a non-leguminous plants (e.g., corn, wheat, and cotton), or an angiosperm or cereal.

The term “isolated” as used throughout the specification is to be understood to mean an agent, for example a protein such a biotin protein ligase, which is purified and/or removed from its natural environment. For example, an isolated biotin protein ligase may be a substantially purified form of the enzyme.

The term “anti-pathogenic agent” as used throughout the specification is to be understood to mean an agent that functions to suppress, destroy, kill, or inhibit the growth, propagation, reproduction or maintenance of an organism. In this regard, the term “survival” of an organism will be understood to encompass the ability of an organism to grow, propagate, reproduce or maintain itself. As will be appreciated, in certain embodiments the term relates to anti-microbial agents, anti-bacterial agents, anti-fungal agents, anti-nematode agents, anti-parasitic agents, and insecticidal agents.

The term “wild type nucleotide sequence” as used throughout the specification is to be understood to mean the nucleotide sequence of a nucleic acid that is present in a naturally occurring organism.

### BRIEF DESCRIPTION OF THE FIGURES

FIG. 1 shows a schematic of the mechanism for attachment of biotin to a lysine residue in a two step reaction catalysed by a biotin protein ligase.

FIG. 2 shows the analysis of pyruvate carboxylase fragments as BPL substrates. Peptides that encompassed the predicted biotin domains of C. albicans and human pyruvate carboxylase were expressed in E. coli as fusions to GST. Expression of each construct was assayed by Western blot using anti-GST antibody and biotinylation determined by Streptavidin blot. The blots used for the analysis of the C-terminal 81 and 108 amino acids of human pyruvate carboxylase are shown in panel A. Quantitation of the blots from three independent experiments is shown in panel B. Similarly, the analysis of three peptides encompassing the predicted biotin domain of C. albicans pyruvate carboxylase is shown in panel C. Protein induction was induced by the addition of isopropyl-β-D-thiogalactopyranoside (IPTG). Uninduced cultures are represented by (−) and induced samples are represented by (+).

FIG. 3 shows the purification of the biotin domain of human pyruvate carboxylase and kinetic analysis of the biotinylation of the domain. Panel A shows purification of the biotin domain of human pyruvate carboxylase. Whole cell lysate from BL21 cells expressing hPC-108 as a fusion to GST were separated on a 1 ml GST-Trap column. Material in the unbound fraction is shown in lane 1 and protein in the wash fraction is shown in lane 2. The biotin domain was released from GST by addition of thrombin directly onto the column. Cleaved material (lane 3) was washed off the column and biotin-containing material removed using Streptavidin Sepharose (lane 4). The purification was monitored by SDS-PAGE (top panel) and Streptavidin blot (lower panel). Migration of molecular mass standards (kDa) is shown on the left and the position of hPC-108 shown on the right. Panel B shows kinetic analysis performed using varied concentrations of purified hPC-108 in an in vitro assay with human BPL. The enzyme displays a low K_{M} for the domain (1±0.2 uM) indicating that it is a good BPL substrate.

FIG. 4 shows in vivo analysis of exogenous BPLs in E. coli BM4062. The E. coli strain BM4062 was transformed with vectors derived from pARA13 (A) for constitutive expression of BPL from S. cerevisiae (B), C. albicans (C) and H. sapiens (D). Strains were grown at the restrictive temperature of 42° C. (left) or the permissive temperature of 30° C. (right).

FIG. 5 shows the purification of C. albicans and human BPL. Recombinant BPL was expressed and purified as described in Example 8. (A) Purification of C. albicans BPL was assessed using SDS-PAGE (top panel) and a Ni-NTA blot to specifically probe for the C-terminal multi-histidine tag (bottom panel). Proteins were fractionated on 12% polyacrylamide gels under reducing conditions. The migration of molecular mass standards is indicated to the left of each panel. Samples for analysis were (1) total protein fraction, (2) soluble protein fraction, (3) unbound fraction, (4) wash fraction, (5) 100 mM imidazole wash fraction and (6) eluted protein. The large arrow indicates the band corresponding to full-length enzyme. The smaller arrow indicates a proteolytic product of the BPL by bacterial proteases. (B) Panel showing the purification of human Met¹-Ala⁸⁰BPL. The arrow represents the expected migration position of this protein.

FIG. 6 shows the kinetic determination of optimal pH for C. albicans BPL activity. The assay for C. albicans BPL activity was performed as described in Example 9. The activity of the enzyme was determined in various buffers in the range of pH 5-10.

FIG. 7 shows the affect of various salts on C. albicans BPL activity. The assay for C. albicans BPL activity was performed as described in Example 9. The activity of the enzyme was determined in the presence of various salt concentrations, as indicated under the graph.

FIG. 8 shows the activity of C. albicans BPL in the presence of various metal ions. The assay for C. albicans BPL activity was performed as described in Example 9. The activity of the enzyme was determined in the presence of various mono- and divalent metal ions, as indicated under the graph.

FIG. 9 shows the activity of C. albicans BPL in the presence of various nucleotide triphosphates. The assay for C. albicans BPL activity was performed as described in Example 9. The activity of the enzyme was determined in the presence of various nucleotide triphosphates, as indicated under the graph.

FIG. 10 shows the activity of C. albicans BPL with varying MgATP concentration. The assay for C. albicans BPL activity was performed as described in Example 9. The activity of the enzyme was determined in the presence of varying concentrations of MgATP, as indicated under the graph.

FIG. 11 shows the activity of C. albicans BPL in the presence of DMSO. The assay for C. albicans BPL activity was performed as described in Example 9. The activity of the enzyme was determined in the presence of varying concentrations of DMSO, as indicated under the graph.

FIG. 12 shows kinetic analysis of the inhibition of C. albicans BPL by pyrophosphate. The assay for C. albicans BPL activity was performed as described in Example 9. The activity of the enzyme was determined in the presence of varying concentrations of pyrophosphate.

FIG. 13 shows the concentration response curve of C. albicans BPL activity inhibition by biotinol-adenylate. The assay for C. albicans BPL activity was performed as described in Example 9. The activity of the enzyme was determined in the presence of varying concentrations of biotinol-adenylate.

FIG. 14 shows the concentration response curve of human BPL activity inhibition by biotinol-adenylate. The assay for human BPL activity was performed as described in Example 9. The activity of the enzyme was determined in the presence of varying concentrations of biotinol-adenylate.

FIG. 15 shows the concentration response curve of bacterial BPL activity inhibition by biotinol-adenylate. The assay for bacterial BPL was performed as described in Example 9. The activity of the enzyme was determined in the presence of varying concentrations of biotinol-adenylate.

FIG. 16 shows the results of inhibition of Candida BPL activity by each of 329 compounds present in a library of test compounds. The activity is expressed relative to control reactions containing DMSO. The graph shows the activity of each compound assayed at 10 μM (grey bars) and 50 μM (black bars).

FIG. 17 shows the differential inhibition of Candida and human BPIs by selected compounds. Compound 69 (Panel A) and Compound 296 (Panel B) were dissolved in 100% DMSO, added to the reactions to a final concentration of 10 μM or 50 μM and their effect on BPL activity measured. The graph shows the average and standard error from reactions performed in triplicate. The inhibitory activity of the compounds was determined relative to the negative control reactions containing only DMSO (“control”). Biotinol-AMP (“BtnOH”) was included in the reactions as a control for inhibition.

FIG. 18 shows the concentration response curve of E. coli, S. aureus, S. cerevisiae and human BPLs by compounds 2 to 5. The assay for enzyme activity was performed as described in Example 4 for the two bacterial enzymes and Example 9 for the yeast and human enzymes. The activity of each enzyme was determined in the presence of varying concentrations of each compound

FIG. 19 shows the antibiotic activity of Compound 2 against Staphylococcus aureus on both solid media (A-C) and in liquid culture (D). The effect on the growth of S. aureus was assessed as described in Example 24. A) DMSO (vehicle control), B) 100 μM ampicillin or C) 100 μM. The clear zone surrounding the filter discs in B & C represents sensitivity of the cells to each treatment. D) Quantitation of the half maximal effective dose (ED50) was performed in liquid culture and determined to be 11.6±1.3 μM.

FIG. 20 shows the toxicity of mammalian liver cells (Hep3B2-1-17) to compounds 2-5. The assay for the compounds was performed as described in Example 25. The toxicity of each compound was determined in the presence of varying concentrations of each compound.

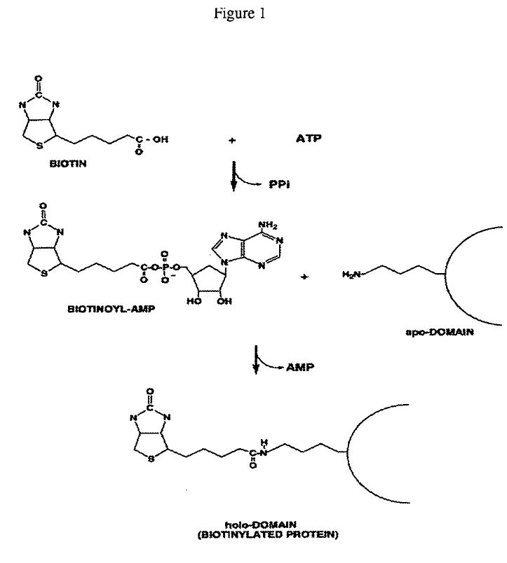

### GENERAL DESCRIPTION OF THE INVENTION

As mentioned above, in one form the present invention provides a method for identifying an inhibitor of a biotin protein ligase, the method including the steps of:
(a) providing a substrate, wherein the substrate may be biotinylated;
(b) contacting the substrate with biotin and a biotin protein ligase in the presence of a test compound;
(c) determining the extent of biotinylation of the substrate by the biotin protein ligase in the presence of the test compound; and
(d) identifying the test compound as an inhibitor of the biotin protein ligase by a reduction in the biotinylation of the substrate in the presence of the test compound as compared to the extent of biotinylation of the substrate in the absence of the test compound.

This form of the present invention is directed to the identification of inhibitors of biotin protein ligase, and in particular, the identification of inhibitors of biotin protein ligases of pathogenic organisms. Inhibitors so identified are candidate compounds for inhibiting biotinylation and as such may be used to inhibit the growth and/or survival of organisms, including the organism from which the biotin protein ligase of interest was derived.

The present invention also provides an inhibitor of a biotin protein ligase identified by the relevant methods for the present invention. As will be appreciated, this form of the present invention may also be used to identify inhibitors of biotinylation of a substrate by a biotin protein ligase.

The biotin protein ligase in the various forms of the present invention may be any biotin protein ligase for which the identification of an inhibitor is desired. Preferably, the biotin protein ligase is a biotin protein ligase of a pathogenic organism of a human, animal or a plant.

Pathogenic organisms of humans or animals for which an inhibitor of a biotin protein ligase may be identified in the various forms of the present invention include bacteria, fungi or parasites. Pathogenic organisms of plants for which an inhibitor of a biotin protein ligase may be identified include bacteria, fungi, insects or nematodes.

Pathogenic bacteria of humans include Acinetobacter calcoaceticus, Acinetobacter lwoffi, Actinobacillus—all species, Actinomadura madurae, Actinomadura pelletieri, Actinomycetaceae—all members, Aeromonas hydrophila, Alcaligenes spp., Arachnia propionica, Arizona spp., Bacillus anthracis, Bacillus cereus, Bacteroides spp., Bartonella—all species, Bordetella—all species, Borrelia—all species, Brucella—all species, Campylobacter coli, Campylobacter fetus, Campylobacter jejuni, Cardiobacterium hominis, Chlamydia psittaci, Chlamydia pneumoniae, Chlamydia trachomatis, Chlamydia psittaci, Clostridium botulinum, Clostridium chauvoei, Clostridium difficile, Clostridium haemolyticum, Clostridium histolyticum, Clostridium novyi, Clostridium perfringens, Clostridium septicum, Clostridium sordellii, Clostridium tetani, Corynebacterium diphtheriae, Corynebacterium equi, Corynebacterium haemolyticum, Corynebacterium pseudotuberculosis, Corynebacterium pyogenes, Corynebacterium renale, Coxiella burnetii, Edwardsiella tarda, Eikenella corrodens, Enterobacter spp., Erysipelothrix rusiopathae (insidiosa), Escherichia coli (enterotoxigenic/invasive/haemorrhagic strains), Flavobacterium meningosepticum, Francisella (Pasteurella) tularensis Type A, Francisella tularensis Type B, Francisella novocida, Haemophilus influenzae, Haemophilus ducreyi, Klebsiella—all species and all serotypes, Helicobacter—all species, Legionella—all species, Leptospira interrogans—all serovars, Listeria—all species, Mimae polymorpha, Moraxella—all species, Morganella morganii, Mycobacterium bovis, Mycobacterium chelonae, Mycobacterium fortuitum, Mycobacterium marinum, Mycobacterium paratuberculosis, Mycobacterium africanum, Mycobacterium avium/intracellulare, Mycobacterium bovis, Mycobacterium kansasii, Mycobacterium leprae, Mycobacterium malmoense, Mycobacterium microtic, Mycobacterium scrofulaceum, Mycobacterium simiae, Mycobacterium szulga, Mycobacterium tuberculosis, Mycobacterium ulcerans, Mycobacterium xenopi, Mycoplasma—all species, Neisseria elongata, Neisseria gonorrhoeae, Neisseria meningitides, Nocardia spp., Pasteurella multocida, Pasteurella. spp., Peptostreptococcus spp., Plesiomonas shigelloides, Porphyromonas spp., Prevotella spp., Proteus—all species, Providencia spp., Pseudomonas aeruginosa, Pseudomonas (Burkholderia) mallei, Pseudomonas (Burkholderia) pseudomallei, Rickettsia—all species, Rhodococcus equi, Salmonella arizonae, Salmonella enteritidis, Salmonella typhimurium, Salmonella paratyphi, Salmonella typhi, Serpulina spp., Serratia liquefaciens, Serratia marcescens, Shigella boydii, Shigella dysenteriae, Shigella flexneri, Shigella sonnei, Shigella dysenteriae, Sphaerophorus necrophorus, Staphylococcus aureus, Stenotrophomonas maltophilia, Streptobacillus moniliformis, Streptococcus spp., Treponema spp., Ureaplasma urealyticum, Vibrio spp., Yersinia (Pasteurella) pestis, Yersinia spp.

Pathogenic fungi of humans include Asperigillus fumigatus, Blastomyces dermatitidis, Candida spp., Cladophialophora bantiana, Coccidioides immitis, Cryptococcus neoformans var. neoformans, Cryptococcus neoformans var. gatii, Emmonsia parva var. parva, Emmonsia para var. crescens, Epidermophyton floccosum, Fonsecaea compacta, Fonsecaea pedrosoi, Histoplasma capsulatum var. capsulatum, Histoplasma capsulatum var. duboisii, Histoplasma capsulatum var. farcinimosum, Madurella grisea, Madurella mycetomatis, Microsporum spp., Neotestudina rosatii, Paracoccidioides brasiliensis, Penicillium marneffei, Scedosporium apiospermum (Pseudallescheria boydii), Scedosporium proliferans (inflatum), Sporothrix schenckii, Trichophyton spp.

Parasites of humans include Acanthamoeba spp., Ancylostoma duodenale, Angiostrongylus cantonensis, Angiostrongylus costaricensis, Anisakis simplex, Ascaris lumbricoides, Ascaris suum, Babesia divergens, Babesia microti, Balantidium coli, Blastocystis hominis, Brugia malayi, Brugia pahangi, Brugia timori, Capillaria spp., Contracaecum osculatum, Cryptosporidium spp., Cyclospora spp., Dicrocoelium dendriticum, Dientamoeba fragilis, Diphyllobothrium latum, Dracunculus medinensis, Echinococcus granulosus, Echinococcus multilocularis, Echinococcus vogeli, Entamoeba histolytica, Enterobius vermicularis, Enterocytozoon bieneusi, Fasciola gigantica, Fasciola hepatica, Fasciolopsis buski, Giardia lamblia (Giardia intestinalis), Heterophyes spp., Hymenolepsis diminuta, Hymenolepsis nana (human origin), Isopora belli, Leishmania brasiliensis, Leishmania donovani, Leishmania spp., Loa loa, Mansonella (Dipetalonema) ozzardi, Mansonella (Dipetalonema) perstans, Mansonella (Dipetalonema) streptocerca, Metagonimus spp., Naegleria spp. (especially fowleri), Necator americanus, Onchocerca volvulus, Opisthorchis spp. (Clonorchis), Paragonimus spp., Plasmodium falciparum, Plasmodium spp., Pneumocystis carinii, Pseudoterranova decipieis, Sarcocystis suihominis, Schistosoma spp., Strongyloides spp., Taenia saginata, Taenia solium, Toxocara canis, Toxocara cati, Toxoplasma gondii, Trichinella nativa, Trichinella nelsoni, Trichinella pseudospiralis, Trichinella spiralis, Trichomonas vaginalis, Trichostrongylus spp., Trichuris trichiura, Trypanosoma brucei rhodesiense, Trypanosoma cruzi, Trypanosoma brucei brucei, Trypanosoma brucei gambiense, Trypanosoma rangeli, Wuchereria bancrofti.

Pathogenic bacteria of plants include Erwinia species, including Erwinia amylovora (Burrill) Winslow et al., Erwinia carotovora (Jones) Bergey et al. subsp. carotovora, Pseudomonas species including Pseudomonas cichorii (Swingle) Stapp, Pseudomonas syringae pv. glycinea (Coerper) Young et al., Pseudomonas syringae pv. pisi (Sackett) Young et al., Pseudomonas syringae pv. syringae van Hall×apple, Pseudomonas syringae pv. syringae van Hall×apricot, Pseudomonas syringae ssp. savastanoi pv. oleae, Xanthomonas species including Xanthomonas campestris pv. campestris (Pammel) Dowson, Xanthomonas campestris pv. corylina Miller et al., Xanthomonas campestris pv. graminis (Egli et al.) Dye, Xanthomonas campestris pv. juglandis (Pierce) Dye, Xanthomonas cynarae Ridé, Xanthomonas fragariae Kennedy et King, Agrobacterium species, and Corynebacterium species.

Pathogenic fungi of plants include Alternaria species, including Alternaria helianthi (Hansf.) Tub. et Nish., Alternaria brassicae (Berk.) Sacc.×Cabbage, Alternaria brassicae (Berk.) Sacc.×Rape, Alternaria brassicicola (Schwein.) Wiltshire, Alternaria dauci (Kühn) Gr. et Sk f. sp. solani (Ell. et Mart.) Neerg., Alternaria radicina (Meier) Drechsler et Eddy; Armilaria species including Armilaria mellea (Vahl) Kummer×grapevine, Armilaria mellea (Vahl) P. Kummer×fig; Ascochyta species including Ascochyta fabae Speg., Ascochyta hortorum (Speg.) Smith, Ascochyta pisi Lib.; Athelia species including Athelia rolfsii (Curzi) Tu et Kimbrough; Botryotinia species including Botryotinia fuckeliana (de Bary) Whetzel×artichoke, Botryotinia fuckeliana (de Bary) Whetzel×blackcurrant, Botryotinia fuckeliana (de Bary) Whetzel×grapevine, Botryotinia fuckeliana (de Bary) Whetzel×hazel, Botryotinia fuckeliana (de Bary) Whetzel×raspberry, Botryotinia fuckeliana (de Bary) Whetzel×strawberry, Botryotinia fuckeliana (de Bary) Whetzel×sunflower, Botrytis fabae Sardina; Bremia species including Bremia lactucae Regel×artichoke, Bremia lactucae Regel×lettuce; Ceratobasidium cereale Murray et Burpee; Cercospora beticola Sacc.; Chondrostereum species including Chondrostereum purpureum (Pers.) Pouzar×apple, Chondrostereum purpureum (Pers.) Pouzar×Prunus sp.; Cladosporium cucumerinum Ell. et Arthur; Claviceps purpurea (Fr.) Tul.; Colletotrichum species including Colletotrichum lagenarium (Pass.) Ellis et Halsted, Colletotrichum trifolii Bain et Essary, Colletotrichum truncatum (Schwein); Coniothyrium diplodiella (Speg.) Sacc.; Corticium solani (Prill. et Delacr.) Bourdot et Galzin, Corticium solani (Prill. et Delacr.) Bourdot et Galzin; Coryneum beijerinckii Oudemans; Cronartium ribicola J. C. Fischer; Cryphonectria parasitica (Murrill) Barr; Cryptosporella viticola (Reddick) Shear; Diaporthe species including Diaporthe cinerescens (Sacc.), Diaporthe eres Nitschke, Diaporthe helianthi Muntanola-Cvetkovic Mihaljcevic et Petrov, Diaporthe phaseolorum var. sojae (Lehman) Wehmeyer; Didymella species including Didymella applanata (Niessl.) Sacc., Didymella lycopersici Klebahn; Diplocarpon earliana (Ell. et Ev.) Wolf; Drepanopeziza ribis (Kleb.) Höhnel; Elsinoe ampelina Shear; Erysiphe species including Erysiphe betae (Vanha) Weltzien, Erysiphe cichoracearum DC.; Erysiphe graminis DC. avenae, Erysiphe polyfaga Hammarlund; Eutypa armeniacae Hansford et Carter; Fulvia fulva (Cooke) Ciferri; Fusarium species including Fusarium oxysporum f. sp. asparagi Cohen, Fusarium oxysporum Schlecht. f. sp. lini (Bolley) Snyd. et Hansen, Fusarium oxysporum Schlecht. f. sp. melonis (L. et C.) Snyd. et Hansen×Cucumber, Fusarium oxysporum Schlecht. f. sp. melonis (L. et C.) Snyd. et Hansen×melon, Fusarium oxysporum Schlecht. f. sp. melonis (L. et C.) Snyd. et Hansen×watermelon, Fusarium oxysporum Schlecht. f. sp. pisi (Linford) Snyd. et Hansen, Fusarium oxysporum Schlecht. f. sp. radicis-lycopersici Jarvis et Shoem., Fusarium roseum (Link) Snyd. et Hansen×asparagus, Fusarium roseum (Link) Snyd. et Hansen×gramineae; Fusicoccum amygdali Delacr.; Glomerella species including Glomerella cingulata (Stoneman) Spaulding et Schrenk×almond, Glomerella cingulata (Stoneman) Spaulding et Schrenk×apple, Glomerella cingulata (Stoneman) Spaulding et Schrenk×olive, Glomerella glycines (Hori) Lehmann et Wolf; Gnomonia leptostyla (Fr.) Ces. et de Not; Guignardia baccae (Cavara) Jacz.; Helicobasidium brebissonii (Desm.) Donk×asparagus, Helicobasidium brebissonii (Desm.) Donk×beet; Kabatiella zeae Narita et Hiratsuka, Leptosphaeria maculans (Desm.) Ces. et de Not.×cabbage; Leptosphaeria species including Leptosphaeria maculans (Desm.) Ces. et de Not.×rape, Leptosphaeria nodorum E. Müller; Leucostoma cinctum (Fr.) Höhnel, Leveillula taurica (Lév.) Arm.; Marssonina panattoniana (Berlese) Magnus; Mastigosporium rubricosum (Dearn. et Barth.) Sprague; Monilinia species including Monilinia fructigena (Aderhold et Ruhl.) Honeye×Whetzel, Monilinia laxa (Aderhold et Ruhland) Honey×Prunus spp.; Monographella nivalis (Schaffnit) E. Müller et v. Arx×cereals; Mycocentrospora cladosporioides (Sacc.) P. Costaex Deighton; Mycoplasme de la Flavescence dorée, Mycosphaerella pinodes (Berk. et Blox.) Vestergren; Mycosphaerella sp.; Nectria galligena Bresad.; Peronospora species including Peronospora destructor (Berk.) Caspary×onion, Peronospora farinosa Fr. f. sp. beta Byford, Peronospora farinosa Fr. f. sp. spinaciae Byford, Peronospora viciae (Berk.) Caspary f. sp. pisi Sydow; Pestalotiopsis menezesiana (Bres. et Torr) Bres. et Torr.; Phakopsora species including Phakopsora euvitis, Phakopsora ampelopsidis, and Phakopsora vitis, Phoma apiicola Klebhan, Phoma exigua Desm., Phoma medicaginis Mal. et Roum v. pinodella (L. K. Jones) Boer.×lucerne; Phoma medicaginis Mal. et Roum v. pinodella (L. K. Jones) Boer.×peas; Phragmidium rubi-idaei (DC.) P. Karsten; Phytophthora species including Phytophthora cactorum (Lebert et Cohn) Schröter, Phytophthora capsici Leonian, Phytophthora fragariae Hickman, Phytophthora infestans (Mont.) de Bary×potato, Phytophthora infestans (Mont.) de Bary×tomato; Plasmopara species including Plasmopara helianthi Novot., Plasmopara viticola (Berk. et Curtisex. de Bary) Berl. et de Toni; Podosphaera leucotricha (Ell. et Ev.) Salmon; Polymyxa betae Keskin; Pseudocercosporella herpotrichoides (Fron) Deighton; Pseudoperonospora species including Pseudoperonospora cubensis (Berk. et Curtis) Rostovtsev, Pseudoperonospora humuli (Miyabe et Takah.) G. Wilson; Pseudopeziza species including Pseudopeziza medicaginis (Lib.) Sacc. f. sp. medicaginis-lupulinae Schmied., Pseudopeziza tracheiphila Müller-Thurgau; Puccinia species including Puccinia allii (DC.) Rudolph, Puccinia asparagi DC., Puccinia coronata Corda, Puccinia coronifera Kleb., Puccinia graminis Persoon f. sp. avenae, Puccinia hordei Otth, Puccinia anomala Rostrup, Puccinia recondita Roberge f. sp. recondita, Puccinia rubigo-vera Winter, Puccinia striiformis Westendorp f. sp. tritici; Pyrenochaeta lycopersici R. Schneider et Gerlach; Pyrenopeziza brassicae B. Sutton et Rawlinson; Pyrenophora species including Pyrenophora graminea Ito et Kuribay, Pyrenophora teres Drechsler; Rhynchosporium secalis (Oudem.) J. Davis; Rosellinia necatrix Prill; Sclerotinia species including Sclerotinia laxa Aderhold et Ruhland, Sclerotinia minor Jagger, Sclerotinia sclerotiorum (Lib.) de Bary×artichoke, Sclerotinia libertiana Fuckel, Sclerotinia sclerotiorum (Lib.) de Bary×carott, Sclerotinia libertiana Fuckel, Sclerotinia sclerotiorum (Lib.) de Bary×rape, Sclerotinia libertiana Fuckel, Sclerotinia sclerotiorum (Lib.) de Bary×rsunflower, Sclerotinia libertiana Fuckel, Sclerotinia sclerotiorum (Lib.) de Bary×soybean, Sclerotinia libertiana Fuckel, Sclerotinia trifoliorum J. Eriksson; Sclerotium bataticola Taub., Sclerotium cepivorum Berk; Septoria apiicola Speg.; Setosphaeria turcica (Luttrell) Leonard et Suggs; Sorosporium reiliana McAlp; Sphacelotheca species including Sphacelotheca reiliania (Kühn) Clinton, Sphaerotheca fuliginea (Schlecht.) Pollacci, Sphaerotheca mors-uvae (Schwein.) Berk. et Curtis; Spilocaea oleagina (Castagne) Hughes; Stereum hirsutum (Willd.) Pers.; Stigmina carpophila (Lév.) M. B. Ellis; Taphrina deformans (Berk.) Tul. var. persicae; Thanatephorus cucumeris (A. B. Frank) Donk×artichoke, Thanatephorus cucumeris (A. B. Frank) Donk×chicory, Uncinula necator (Schw.) Burr.; Urocystis cepulae Frost; Uromyces betae Kickx, Ustilago maydis (DC.) Corda; Venturia inaequalis (Cooke) Winter, Venturia pirina Aderhold; Verticillium species including Verticillium albo-atrum Reinke et Berthold×lucerne, Verticillium albo-atrum Reinke et Berthold×sunflower, Verticillium dahliae Klebahn×artichoke, Verticillium dahliae Klebahn×strawberry, Verticillium dahliae Klebahn×sunflower, Verticillium dahliae Klebahn×tomato.

Pathogenic insects of plants include insect pests of the orders of Lepidoptera, Coleoptera, Diptera, Homoptera, Hemiptera, Thysanoptera, and Orthoptera.

Pathogenic nematodes of plants include nematodes of the genera Heterodera, Globodera, Umbelliferae, Solanaceae, Pratylinchus, and Meloidogynze.

In one preferred embodiment, the biotin protein ligase is a biotin protein ligase of a pathogenic organism of a human. Preferably, the biotin protein ligase is a biotin protein ligase from a fungal, bacterial or parasitic pathogen of a human.

In the case of a bacterial pathogen of a human, preferably the biotin protein ligase is a biotin protein ligase from Escherichia coli or Staphylococcus aureus.

In the case of a fungal pathogen of a human, preferably the biotin protein ligase is a biotin protein ligase from a Candida species, such as Candida albicans, or a yeast.

In another preferred embodiment, the biotin protein ligase is a biotin protein ligase of a pathogenic organism of a plant. Preferably, the biotin protein ligase is a biotin protein ligase from a fungal, bacterial, insect or nematode pathogen of a plant.

The present invention also provides the use of a biotin protein ligase as a target for identifying agents that inhibit growth of an organism. Preferred biotin protein ligases from various organisms are as herein described.

In another form, the present invention provides a method for identifying an agent that inhibits growth and/or survival of an organism, the method including the step of identifying an agent that inhibits a biotin protein ligase.

In another form, the present invention provides a biotin protein ligase suitable for use as a target for identifying an inhibitor of biotinylation, and/or suitable for use as a target for identifying an inhibitor of growth and/or survival of an organism. Preferably, the biotin protein ligase is an isolated biotin protein ligase. Methods for isolating biotin protein ligases are known in the art.

In a particularly preferred form, the present invention provides an isolated biotin protein ligase when used as a target for identifying an inhibitor of biotinylation, and/or when used as a target for identifying an inhibitor of growth and/or survival of an organism. Examples of biotin protein ligases particularly suitable for the present invention and their associated sequence data are as follows:

### Nematodes:

gi|22532948| Caenorhabditis elegans
gi|39597053| Caenorhabditis briggsae

### Mammals:

gi|1705499| Homo sapiens
gi|55655926| Pan troglodytes
gi|74001437| Canis familiaris
gi|20982837| Mus musculus

### Birds:

gi|50729961| Gallus gallus

### Fish:

gi|47223799| Tetraodon nigrroviridis

### Plants:

gi|50905509| Oryza sativa
gi|6623977| Arabidopsis thaliana

### Fungi:

gi|66852927| Aspergillus fumigatus
gi|49097464| Aspergillus nidulans
gi|46127903| Fusarium graminearinearum
gi|32409781| Neurospora crassa
gi|50289965| Candida glabrata
gi|1345625| Saccharomyces cerevesiae
gi|50310579| Kluyveromyces lactis
gi|45187958| Eremothecium gossypii
gi|50554375| Yarrowia lipolytica
gi|50410019| Debaryomyces hansenii
gi|68473197| Candida albicans
gi|19113070| Schizosaccharomyces pombe
gi|46099450| Ustilago maydis
gi|57225614| Cryptococcus neoformans

### Insects:

gi|23170360| Drosophila melanogaster
gi|66565539| Apis mellifera
gi|58396066| Anopheles gambiae

### Microbial:

gi|29377684| Enterococcus faecalis
gi|669248979| Enterococcus faecalis
gi|77410716| Streptococcus agalactiae
gi|71853705| Streptococcus pyogenes
gi|55823094| Streptococcus thermophilus
gi|50590792| Streptococcus suis
gi|15459392| Streptococcus pneumoniae
gi|66877104| Streptococcus pneumoniae
gi|25528935| Lactococcus lactis
gi|62464640| Lactococcus lactis
gi|18309655| Clostridium
gi|28270529| Lactobacillus plantarum
gi|48865543| Oenococcus oeni
gi|53689281| Leuconostoc mesenteroides
gi|15673762| Lactococcus
gi|48870974| Pediococcus pentosaceus
gi|27315607| Staphylococcus epidermidis
gi|72495112| Staphylococcus saprophyticus
gi|49241774| Staphylococcus aureus
gi|15924446| Staphylococcus aureus
gi|20808369| Thermoanaerobacter tengcongensis
gi|76796777| Thermoanaerobacter ethanolicus
gi|25500437| Clostridium acetobutylicum
gi|28203006| Clostridium tetani
gi|67873832| Clostridium thermocellum
gi|25329523| Clostridium acetobutylicum
gi|20089561| Methanosarcina acetivorans
gi|20906352| Methanosarcina mazei
gi|12018159| Methanosarcina barkeri
gi|68211758| Methanococcoides burtonii
gi|15679898| Methanothermobacter thermautotrophicus
gi|1168672| Bacillus subtilis
gi|52080752| Bacillus licheniformis
gi|29895229| Bacillus cereus
gi|75760405| Bacillus thuringiensis
gi|65318902| Bacillus anthracis
gi|47566000| Bacillus cereus
gi|52143811| Bacillus cereus
gi|49332554| Bacillus thuringiensis
gi|56420715| Geobacillus kaustophilus
gi|25293806| Bacillus halodurans
gi|25517390| Listeria innocua
gi|25517191| Listeria monocytogenes
gi|22777448| Oceanobacillus iheyensis
gi|56963833| Bacillus clausii
gi|68009872| Exiguobacterium
gi|71547835| Syntrophobacter fumaroxidans
gi|73748653| Dehalococcoides
gi|57234388| Dehalococcoides ethenogenes
gi|76802823| Natronomonas pharaonis
gi|55229668| Haloarcula marismortui
gi|16554497| Halobacterium
gi|66858469| Anaeromyxobacter dehalogenans
gi|69260263| Magnetococcus
gi|68270618| Moorella thermoacetica
gi|71740435| Pelobacter propionicus
gi|68178684| Desulfuromonas acetoxidans
gi|71545931| Syntrophobacter fumaroxidans
gi|68549351| Pelodictyon phaeoclathratiforme
gi|67936171| Chlorobium phaeobacteroides
gi|67917999| Chlorobium limicola
gi|68552171| Prosthecochloris aestuarii
gi|67915810| Chlorobium phaeobacteroides
gi|71481333| Prosthecochloris vibrioformis
gi|14520365| Pyrococcus abyssi
gi|14590088| Pyrococcus horikoshii
gi|18976449| Pyrococcus furiosus
gi|57640922| Thermococcus kodak
gi|71541889| Syntrophomonas wolfei
gi|55980194| Thermus thermophilus
gi|46200063| Thermus thermophilus
gi|66798302| Deinococcus geothermalis
gi|11497694| Archaeoglobus fulgidus
gi|34541252| Porphyromonas gingivalis
gi|48856040| Cytophaga hutchinsonii
gi|62389598| Corynebacterium glutamicum
gi|19551939| Corynebacterium glutamicum
gi|25027296| Corynebacterium efficiens
gi|38199527| Corynebacterium diphtheriae
gi|68264350| Corynebacterium jeikeium
gi|15610415| Mycobacterium tuberculosis
gi|31794459| Mycobacterium bovis
gi|13092858| Mycobacterium leprae
gi|15827309| Mycobacterium leprae
gi|54022962| Nocardia farcinica
gi|69288625| Kineococcus radiotolerans
gi|29606981| Streptomyces avermitilis
gi|72162952| Thermobifida fusca
gi|71369273| Nocardioides
gi|66964306| Arthrobacter
gi|50954284| Leifsonia xyli
gi|68229616| Frankia
gi|62423517| Brevibacterium linens
gi|23326815| Bifidobacterium longum
gi|46190507| Bifidobacterium longum
gi|59802309| Neisseria gonorrhoaea
gi|11353875| Neisseria meningitidis
gi|30043000| Shigella flexneri
gi|75176639| Shigella boydii
gi|115015| Escherichia coli
gi|17865658| Salmonella typhimurium LT2
gi|56415970| Salmonella enterica
gi|25293809| Yersinia pestis
gi|50119174| Erwinia carotovora
gi|37528549| Photorhabdus luminescens
gi|37681353| Vibrio vulnificus
gi|27364604| Vibrio vulnificus
gi|75854775| Vibrio
gi|75828714| Vibrio cholerae
gi|59713032| Vibrio fischeri
gi|76793426| Pseudoalteromonas atlantica
gi|69163060| Shewanella frigidimarina
gi|69942916| Shewanella denitrificans
gi|68548555| Shewanella amazonensis
gi|68544816| Shewanella baltica
gi|56461103| diomarina loihiensis
gi|23468178| Haemophilus somnus
gi|1168673| Haemophilus influensae
gi|75430066| Actinobacillus succinogenes
gi|32034277| Actinobacillus pleuropneumoniae
gi|33147568| Haemophilus
gi|11272589| Xylella fastidiosa
gi|21115195| Xanthomonas campestris
gi|21110428| Xanthomonas axonopodis
gi|77163726| Nitrosococcus oceani
gi|30250381| Nitrosomonas europaea
gi|71550332| Nitrosomonas eutropha
gi|68214112| Methylobacillus flagellatus
gi|74316478| Thiobacillus denitrificans
gi|34495941| Chromobacterium violaceum
gi|56477766| Azoarcus
gi|71848990| Dechloromonas aromatica
gi|66047791| Pseudomonas syringae
gi|70728946| Pseudomonas fluorescens
gi|67156990| Azotobacter vinelandii
gi|48860573| Microbulbifer degradans
gi|67676689| Chromohalobacter salexigens
gi|33591356| Bordetella pertussis
gi|3800713| Bordetella pertussis
gi|33594878| Bordetella parapertussis
gi|33575199| Bordetella bronchiseptica
gi|74021420| Rhodoferax ferrireducens
gi|47573914| Rubrivivax gelatinosus
gi|52208452| Burkholderia pseudomallei
gi|52428035| Burkholderia malle
gi|74014039| Burkholderia ambifaria
gi|67668254| Burkholderia cenocepacia
gi|67546636| Burkholderia vietnamiensis
gi|48786400| Burkholderia fungorum
gi|73539808| Ralstonia
gi|68554774| Ralstonia metallidurans
gi|17545031| Ralstonia solanacearum
gi|17987427| Brucella melitensis
gi|62289765| Brucella abortus
gi|23501703| Brucella suis
gi|23347626| Brucella
gi|69277800| Mesorhizobium
gi|13471392| Mesorhizobium
gi|74420934| Nitrobacter winogradskyi
gi|69140916| Nitrobacter hamburgensis
gi|39649855| Rhodopseudomonas palusiris
gi|49475644| Bartonella henselae
gi|49239695| Bartonella quintana
gi|15965033| Sinorhizobium meliloti
gi|69298187| Silicibacter
gi|56679337| Silicibacter pomeroyi
gi|77387506| Rhodobacter sphaeroides
gi|69152482| Paracoccus
gi|231637| Paracoccus denitrificans
gi|68182893| Jannaschia
gi|16126179| Caulobacter cresce
gi|13423393| Caulobacter crescentus
gi|48764633| Rhodospirillum rubrum
gi|23014039| Magnetospirillum magnetotacticum
gi|61101006| Erythrobacter litoralis
gi|68539856| Sphingopyxis alaskensis
gi|56544338| Zymomonas obilis
gi|58038571| Gluconobacter oxydans
gi|70866719| Trypanosoma cruzi
gi|70834456| Trypanosoma brucei
gi|25534830| Nostoc
gi|17232748| Nostoc
gi|53764434| Anabaena variabilis
gi|23126331| Nostoc punctiforme
gi|71675719| Trichodesmium erythraeum
gi|67856925| Crocosphaera watsonzii
gi|7469268| Syneclocystis
gi|56751177| Synechococcus elongatus
gi|37520907| Gloeobacter violaceus
gi|62515609| Lactobacillus delbrueckii
gi|62513575| Lactobacillus casei
gi|7520946| Syphilis spirochete
gi|15639348| Treponema pallidum
gi|16752173| Chlamydophila pneumoniae
gi|29835007| Chlamydophila caviae
gi|76167995| Chlamydia trachomatis
gi|23509795| Plasmodium falciparum
gi|23482140| Plasmodium yoelii
gi|56499526| Plasmodium berghei
gi|46227802| Cryptosporidium parvum
gi|41016764| Methanocaldococcus jannaschii
gi|45357682| Methanococcus maripaludis
gi|7514332| Aquifex aeolicus
gi|51460027| Rickettsia typhi
gi|15604390| Rickettsia prowazekii
gi|15892696| Rickettsia conorii
gi|42453815| Rickettsia rickettsii
gi|67004669| Rickettsia felis
gi|52698978| Rickettsia akari
gi|58417546| Ehrlichia ruminant
gi|58616979| Ehrlichia ruminant
gi|73666871| Ehrlichia canis
gi|58419193| Wolbachia
gi|52628131| Legionella pneumophila
gi|33517318| Blochmannia floridanus
gi|71891971| Blochmannia pennsylvanicus
gi|33239881| Prochlorococcus marinus
gi|33633186| Synechococcus
gi|70607375| Sulfolobus acidocaldarius
gi|15897572| Sulfolobus solfataricus
gi|57237106| Campylobacter jejuni
gi|57504657| Campylobacter coli
gi|57505335| Campylobacter upsaliensis
gi|57240720| Campylobacter lari
gi|71151002| Thiomicrospira denitrificans
gi|15645754| Helicobacter pylori
gi|46445799| Parachlamydia
gi|10580250| Halobacterium
gi|34762115| Fusobacterium nucleatum
gi|67929504| Solibacter usitatus
gi|48764875| Rhodospirillum rubrum
gi|71083596| Pelagibacter ubique
gi|72009746| Strongylocentrotus purpuratus
gi|28492991| Tropheryma whipple
gi|76261556| Chloroflexus
gi|56707619| Francisella tularensis
gi|46580244| Desulfovibrio vulgaris
gi|49530030| Acinetobacter
gi|29250335| Giardia lamblia
gi|67480425| Entamoeba histolytica
gi|15246938| Desulfotalea psychrophila

Preferably, the use of a biotin protein ligase as a target for identifying agents that inhibit growth of an organism includes the use of one of the above biotin protein ligases.

Under conditions when inhibitors are identified in vitro, the biotin protein ligase of interest for use in the methods according to the present invention may be purified by a suitable method known in the art, including the purification of the protein from cell extracts from the organism of interest, or the expression of the protein in an appropriate expression system from an appropriate expression vector having the cloned gene. In the latter case, the biotin protein ligase may be expressed with an additional moiety that allows the enzyme to be readily purified. Examples of such additional moieties include glutathione S transferase (GST), histidine tags, maltose binding proteins, and calmodulin binding peptides. Such an additional moiety may or may not be removed from the biotin protein ligase before use.

The biotin protein ligase may be purified to a desired extent so as to allow the inhibition of the extent of biotinylation of the substrate by the ligase to be determined.

Under conditions when inhibitors are identified in vivo, the biotin protein ligase of interest for use in the methods according to the present invention may be the endogenous biotin protein ligase, or alternatively, may be a cloned biotin protein ligase expressed in a cell.

The substrate according to the various forms of the invention is any molecule that has the capacity to have a biotin group covalently attached to the molecule by the action of a biotin protein ligase. Examples of suitable substrates include biotin-containing proteins such as pyruvate carboxylase, acetyl CoA carboxylase, propionyl CoA carboxylase, B-methylcrotonyl CoA carboxylase, methylmalonyl-CoA carboxyltransferase, oxaloacetate decarboxylase, methylmalonyl-CoA decarboxylase, glutaconyl-CoA decarboxylase, urea carboxylase, geranoyl-CoA carboxylase and geranoyl-CoA transcarboxylase, or a functional variant of any of the aforementioned proteins; a polypeptide including the biotin domain of any of the aforementioned protein substrates or variants of the proteins; proteins or polypeptides synthesised in vitro by chemical synthesis or in vitro translation that have the capacity to be biotinylated; or a small molecule that has the ability to have a free biotin group covalently attached to the molecule by a BPL, such as hydroxylamine.

Preferably, the substrate is a polypeptide fragment containing the biotin domain of a biotin-containing protein or hydroxylamine. Most preferably, the substrate is a polypeptide fragment containing the biotin domain of a biotin-containing protein.

Methods for identifying proteins encoding examples of the substrates hereinbefore described may be achieved by a suitable method. For example, pyruvate carboxylases may be identified using the BLAST algorithm, which determines the extent of homology between two nucleotide sequences (blastn) or the extent of homology between two amino acid sequences (blastp). BLAST identifies local alignments between the sequences in the database and predicts the probability of the local alignment occurring by chance. The BLAST algorithm is as described in Altschul et al. (1990) J. Mol. Biol. 215:403-410.

Under conditions when inhibitors are identified in vitro, preferably the substrate is a protein substrate derived from the same species or genus as that of the biotin protein ligase of interest. For example, when identifying inhibitors of Candida biotin protein ligase, the substrate is preferably a polypeptide from a Candida biotin carboxylase, such as Candida albicans pyruvate carboxylase. However, it will be appreciated that the substrate may also be derived from an unrelated species or genus as that of the biotin protein ligase of interest.

In the case of the identification of inhibitors of Candida biotin protein ligase in vitro, the substrate is preferably pyruvate carboxylase or a polypeptide fragment derived from Candida albicans pyruvate carboxylase, or a functional variant thereof. Sequence data for Candida albicans pyruvate carboxylase may be obtained from the Stanford Genonie Technology Centre (orf6.2989 orf6-2189: 1646-4594).

Most preferably, the protein substrate includes (or consists of) one of the following polypeptides from Candida albicans pyruvate carboxylase (or a functional variant or thereof):

caPC-115:

(SEQ ID NO.1)

M⁸⁶⁸AVGDVSEKTGTREVFFELNGEMRSVSVEDKTVSVELKTRPKASASN
EVGAPMAGVVIEIRAHKHQQIAKGDPIAVLSAMKMEMVISAPCSGEIGDI
LIHEGDSVDANDLITSIH⁹⁸².
caPC-93:

(SEQ ID NO.2)

M⁸⁹⁰RSVSVEDKTVSVELKTRPKASASNEVGAPMAGVVIEIRAHKHQQIA
KGDPIAVLSAMKMEMVISAPCSGEIGDILIHEGDSVDANDLITSIH⁹⁸².
caPC-74:

(SEQ ID NO.3)

K⁹⁰⁹ASASNEVGAPMAGVVIEIRAHKHQQIAKGDPIAVLSAMKMEMVISA
PCSGEIGDILIHEGDSVDANDLITSIH⁹⁸².

In the case of the identification of inhibitors of an insect biotin protein ligase in vitro, the substrate is preferably pyruvate carboxylase or a polypeptide fragment derived from a Drosophila pyruvate carboxylase (gi|19921944|ref|NP_{—}610527.1| BcDNA:GH06348 gene product), or a functional variant thereof. Most preferably, the protein substrate includes the following polypeptide from Drosophila melanogaster pyruvate carboxylase (or a functional variant thereof):

dPC-125:

(SEQ ID NO.4)

G¹⁰⁵⁷KTLSVKALAVSADLKPNGIREVFFELNGQLRAVHILDKEAVKEIH
VHPKANKSNKSEVGAPMPGTVIDIRVKVGDKVEKGQPLVVLSAMKMEMVV
QSPLAGVVKKLEIANGTKLEGEDLIMIIE¹¹⁸¹.

In the case of the identification of an inhibitor of a bacterial biotin protein ligase in vitro, the substrate is preferably pyruvate carboxylase or a polypeptide derived from E. coli acetyl CoA carboxylase, or a functional variant thereof (gi|1789653|gb|AAC76287.1|[1789653]). Most preferably, the protein substrate includes the following polypeptide from E. coli acetyl CoA carboxylase (or a functional variant thereof):

BCCP-87:

(SEQ ID NO.5)

M⁷⁰EAPAAAEISGHIVRSPMVGTFYRTPSPDAKAFIEVGQKVNVGDTLCI
VEAMKMMNQIEADKSGTVKAILVESGQPVEFDEPLVVIE¹⁵⁶.

Additional polypeptides that may be used as protein substrates in vitro for E. coli BPL include the following polypeptides:

| | |
|---|---|
| LGGIFEAMKMELRD; | (SEQ ID NO.6) |
| LFLHDFLNAQKVELYPVTSSG; | (SEQ ID NO.7) |
| MAGGLNDIFEAQKIEWHEDTGGS; | (SEQ ID NO.8) |

or a functional variant of these polypeptides.

As will be appreciated, under conditions when inhibitors are identified in vitro, the substrate of interest for use in the methods according to the present invention will be present at such a concentration and such a level of purity to allow biotinylation of the substrate by the biotin protein ligase to be measured.

In the case of the substrate being a protein substrate, the protein substrate may be purified by a suitable method known in the art, including the purification of the protein from cell extracts from the organism of interest, or the expression of the protein in an appropriate expression system from an appropriate expression vector having the cloned nucleotide sequence. The protein substrate may be expressed with an additional moiety that allows the protein substrate to be readily purified. Examples of such additional moieties include glutathione S transferase (GST), histidine tags, maltose binding proteins, and calmodulin binding peptides. Such an additional moiety may or may not be removed from the biotin protein ligase before use. As discussed, the protein substrate will be purified to an extent to allow the inhibition of the extent of biotinylation of the protein substrate by the ligase to be determined.

Contacting the substrate with biotin and the biotin protein ligase in the various forms of the present invention may be performed under any suitable conditions that allow the substrate to be biotinylated by the biotin protein ligase. Under conditions when inhibitors are identified in vitro, preferably the contacting of the substrate with biotin and biotin protein ligase occurs when the substrate is directly or indirectly coupled to a solid phase. In this regard, it has been determined that the ability to identify inhibitors of biotin protein ligases is improved by contacting the substrate with biotin and a biotin protein ligase when the substrate is coupled to a solid phase.

Examples of direct coupling include direct adsorption of the substrate to a solid phase, or direct covalent attachment of the substrate to the solid phase utilising the reaction of a reactive chemical moiety on the column with a reactive moiety on the substrate, with or without a linker or spacer. Preferably, the direct coupling is by way of direct adsorption to a solid phase.

Indirect coupling involves the coupling of the substrate to the solid phase using a molecule or moiety attached to the solid phase that has the ability to capture the substrate non-covalently. Examples of indirect coupling include the coupling of an antibody to the substrate to the solid phase and capturing the substrate (ie antigen) with the antibody, or the capturing of a six histidine sequence by a Nickel column.

In the case where the contacting occurs in vitro, the concentration of each of the substrate, biotin and biotin protein ligase in the reaction will be selected so as to allow sufficient biotinylation of the substrate to occur to allow for the determination of whether a test compound at a particular concentration has the capacity to inhibit biotinylation of the substrate.

In the case where the contacting occurs in vivo, the contacting may occur in a cell type that allows the extent of biotinylation of the substrate by the biotin protein ligase to be determined. The biotin protein ligase may be present endogenously in the cell, or alternatively, may be expressed for example from an introduced nucleic acid. Similarly, the substrate may either be expressed endogenously by the cell or be expressed from an introduced nucleic acid. In some cases, the biotin protein ligase and/or the substrate may also be contacted with the cell so as to enter the cell.

In this regard, methods for the isolation of nucleic acid sequences and their cloning into a suitable expression vector are essentially as described in Sambrook, J, Fritsch, E. F. and Maniatis, T. Molecular Cloning: A Laboratory Manual 2nd. ed. Cold Spring Harbor Laboratroy Press, New York. (1989). The recombinant molecule may then be introduced into the cell by a method known in the art and the cloned nucleic acid expressed.

Methods for introducing nucleic acids into cells and expressing proteins in the various forms of the present invention, are known in the art. For example, a nucleic acid may be introduced into a cell by various methods, including transformation using calcium phosphate, viral infection, electroporation, lipofection, and particle bombardment. Methods for introducing DNAs into cells are essentially as described in Sambrook, J, Fritsch, E. P. and Maniatis, T. Molecular Cloning: A Laboratory Manual 2nd. ed. Cold Spring Harbor Laboratory Press, New York. (1989).

Under conditions when inhibitors are identified in vitro, the biotinylation reaction is dependent upon the presence of a suitable divalent metal ion. Preferably, the divalent metal ion is a magnesium ion or calcium ion. Most preferably, the divalent metal ion is a magnesium ion. The concentration of the divalent metal ion is preferably in the range of 0.5 to 10 mM.

The biotinylation reaction is also dependent upon the presence of ATP. Preferably, the concentration of ATP is in the range of 0.5 uM to 5 mM. Most preferably, the concentration of ATP is 100 uM.

Under conditions when inhibitors are identified in vitro, the biotinylation reaction may be buffered by any suitable buffer, including acetate, carbonate or Tris buffers, and the pH of the reaction may be within a suitable range to allow biotinylation of the substrate by the biotin protein ligase. Preferably, die pH of the reaction is in the range of 7.0 to 8.5. Most preferably, the pH of the reaction is in the range from 7.5 to 8.0.

Under conditions when inhibitors are identified in vitro, the biotinylation reaction may be performed at a suitable temperature and for a suitable time, and may also include the presence of a salt and/or a reducing agent. Similarly, under conditions when inhibitors are identified in vivo, the test compound will be contacted with cells for an appropriate time to allow inhibition of biotinylation to be determined.

The test compound may be any compound that may act as a potential inhibitor of the biotinylation of the protein substrate. For example, the test compound may be a small molecule, drug, protein, peptide, polypeptide, antibody or antigen binging portion thereof, polysaccharide, glycoprotein, lipid, metabolite, cofactor, transition state analogue, nucleotide, nucleotide analogue, or nucleic acid. Under conditions when inhibitors are identified in vivo, the test compound will need to be able to enter the cell and have the capacity to exert an inhibitory action on the biotin protein ligase. Alternatively, the test compound may be expressed in the cells, such as for the case of a potentially inhibitory polypeptide.

As will be appreciated, the ability of the test compound to inhibit biotinylation of the protein substrate will depend on the concentration of the test molecule. Accordingly, the concentration of the test molecule will be selected so as to determine the ability of the test molecule to inhibit biotinylation of the protein substrate at that concentration.

For in vitro purposes, to separate biotinylated substrate from free biotin, the substrate may be partitioned from free biotin by washing the substrate bound to a solid phase under conditions to remove free biotin. Alternatively, free biotin may be removed from the substrate by the preferential binding of the free biotin to a solid phase, such as the binding of biotin to anion exchange resin. Preferably, biotinylated substrate is partitioned from free biotin by the coupling of the substrate to a solid phase and removing free biotin by washing of the solid phase.

The determination of the extent of biotinylation of the substrate in the various forms of the present invention may be performed by a suitable method known in the art.

For example, under conditions when inhibitors are identified in vitro, biotinylation of the substrate may be detected by use of detectably labelled streptavidin or avidin. Alternatively, detectably labelled biotin may be employed. The extent of biotinylation of the substrate in the presence and absence of the test compound may then be determined, and the test compound identified as an inhibitor of the biotin protein ligase by a reduction in the extent of biotinylation of the substrate in the presence of the test compound as compared to the extent of biotinylation in the absence of the test compound.

For detection of the extent of biotinylation of the protein substrate, the presence of biotin may be detected by any suitable method that allows the determination of the extent of biotinylation of the protein substrate. For example, detectably labelled streptavidin may be utilised.

Under conditions when inhibitors are identified in vivo, a protein substrate may be detected by preparing a cell lysate, isolating one or more protein substrates and determining the extent of biotinylation of the protein substrates. For example, the cell lysate may be fractioned by SDS-PAGE, the protein detected with a suitable antibody and the extent of biotinylation determined by a streptavidin blot. Alternatively, one or more protein substrates may be immunoprecipitated and the extent of biotinylation determined by detectably labeled streptavidin or avidin.

Identification of the test compound as an inhibitor of a biotin protein ligase will be made by a comparison of the extent of biotinylation of the substrate in the presence and absence of the test compound. Preferably, the maximum extent of inhibition of activity by the test compound is greater than 70%. More preferably, the maximum extent of inhibition of activity is greater than 90%.

Preferably, the selection of a suitable compound as a test compound is first made by the use of an in silico selection method.

Preferably, the in silico method utilises the structure of a biotin protein ligase to identify a potential test compound. More preferably, the in silico method utilises the structure of the biotin protein ligase in the absence and presence of biotin to identify potential test compounds.

For example, the X-ray structures of BPL from Escherichia coli, in the absence (Wilson et al (1992) “Escherichia coli biotin holoenzyme synthetase/bio repressor crystal structure delineates the biotin- and DNA-binding domains” Proc. Natl. Acad. Sci. USA. 89; 9257-9261) and presence of biotin (Weaver et al (2001) “Corepressor-induced organization and assembly of the biotin repressor: A model for the allosteric activation of a transcriptional regulator” Proc Natl. Acad. Sci. USA. 98; 6045-6050) may be used to identify potential inhibitory compounds using in silico technology. Missing amino acids and hydrogen atoms in the structure (PDB: IBIB) may be modelled using the Biopolymer module of SYBYL v7.0 (Tripos, Inc. SYBYL, Version 7.0, St. Louis, Mo. 2004). Modelled sections of the protein may be refined by conjugate gradient energy minimisation in explicit water using the program NAMD (Linge et al (2003) “Refinement of protein structures in explicit solvent” Proteins 50; 496-506; Kale et al. (1999) “NAMD2: greater scalability for parallel molecular dynamics” J. Comput. Phys. 151: 283-312). Partial charges for BPL and the Jul. 24, 2004 compounds of interest, for example as present in a compound library (eg ZINC) may be calculated using Biopolymer and the SYBYL Programming Language (SPL) respectively. A negative image of the active sites for the prepared BirA may be constructed with ATPTS 2001 (Moreno et al. (2002) “Geometric and chemical patterns of interaction in protein—ligand complexes and their application in docking” Proteins 47: 1-13). The compound library may then be docked with DOCK v5.1.0. (Ewing et al. (2001) “DOCK 4.0: search strategies for automated molecular docking of flexible molecule databases” J. Comput. Aided Mol. Des. 15: 411-428). Poses may be scored using Scorer v1.3 and ranked with a threshold of 10% in an in-house consensus scoring program based on the CScore module of SYBYL. Compounds with a score of 6 and the top compounds based on the internal Dock Energy Score may be visually inspected and non-viable candidates filtered out.

Potential test compounds selected by the in silico methodology may then be screened for their ability to inhibit biotinylation as previously herein described.

As described previously, the present invention also provides an inhibitor of a biotin protein ligase identified according to the relevant methods for the present invention.

For example, the present invention may be used to identify an inhibitor of a biotin protein ligase of a pathogenic organism.

Accordingly, in another form the present invention provides an anti-pathogenic agent, wherein the agent inhibits biotinylation of a substrate by a biotin protein ligase of a pathogen.

In another form, the present invention provides an inhibitor of a biotin protein ligase of a pathogenic organism. Examples of pathogenic organisms and their biotin protein ligases are as previously hereinbefore described.

For example, the present invention may be used to identify an inhibitor of a bacterial biotin protein ligase or an inhibitor of a microbial protein ligase.

Accordingly, in another form the present invention provides an inhibitor of a bacterial biotin protein ligase.

In another form, the present invention provides an anti-bacterial agent, wherein the agent inhibits biotinylation of a substrate by a bacterial biotin protein ligase.

Preferably, the anti-bacterial agent is an inhibitor of (and inhibits biotinylation of a substrate by) a biotin protein ligase from a bacterium selected from the group consisting the genus or species of Enterococcus faecalis, Streptococcus agalactiae, Streptococcus pyogenes, Streptococcus thermophilus, Streptococcus suis, Streptococcus pneumoniae, Lactococcus lactis, Clostridium, Lactobacillus plantarum, Oenococcus oeni, Leuconostoc mesenteroides, Lactococcus pentosaceus, Staphylococcus epidermidis, Staphylococcus saprophyticus, Staphylococcus aureus, Thermoanaerobacter tengcongensis, Thermoanaerobacter ethanolicus, Clostridium acetobutylicum, Clostridium tetani, Clostridium thermocellum, Clostridium acetobutylicum, Methanosarcina acetivorans, Methanosarcina mazei, Methanosarcina barkeri, Methanococcoides burtonii, Methanothermobacter thermautotrophicus, Bacillus subtilis, Bacillus licheniformis, Bacillus cereus, Bacillus thuringiensis, Bacillus anthracis, Bacillus cereus, Geobacillus kaustophilus, Bacillus halodurans, Listeria innocua, Listeria monocytogenes, Oceanobacillus iheyensis, Bacillus clausii, Exiguobacterium, Syntrophobacter fumaroxidans, Dehalococcoides including Dehalococcoides ethenogenes, Natronomonas pharaonis, Haloarcula marismortui, Halobacterium, Anaeromyxobacter dehalogenans, Magnetococcus, Moorella thermoacetica, Pelobacter propionicus, Desulfuromonas acetoxidans, Syntrophobacter fumaroxidans, Pelodictyon phaeoclathratiforme, Chlorobium phaeobacteroides, Chlorobium limicola, Prosthecochloris aestuarii, Chlorobium phaeobacteroides, Prosthecochloris vibrioformis, Pyrococcus abyssi, Pyrococcus horikoshii, Pyrococcus furiosus, Thermococcus Kodak, Syntrophomonas wolfei, Thermus thermophilus, Deinococcus geothermalis, Archaeoglobus fulgidus, Porphyromonas gingivalis, Cytophaga hutchinsonii, Corynebacterium glutamicum, Corynebacterium glutanicum, Corynebacterium efficiens, Corynebacterium diphtheriae, Corynebacterium jeikeium, Mycobacterium tuberculosis, Mycobacterium bovis, Mycobacterium leprae, Nocardia farcinica, Kineococcus radiotolerans, Streptomyces avermitilis, Thermobifida fusca, Nocardioides, Arthrobacter, Leifsonia xyli, Frankia, Brevibacterium linens, longum, Bifidobacterium longum, Neisseria gonorrhoaea, Neisseria meningitides, Shigella flexneri, Shigella boydii, Escherichia coli, Salmonella typhimurium LT2, Salmonella enterica, Yersinia pestis, Erwinia carotovora, Photorhabdus luminescens, Vibrio vulnificus, Vibrio vulnificus, Vibrio, Vibrio cholerae, Vibrio fischeri, Pseudoalteromonas atlantica, Shewanella frigidimarina, Shewanella denitrificans, Shewanella amazonensis, Shewanella baltica, diomarina loihiensis, Haemophilus somnus, Haemophilus influensae, Actinobacillus succinogenes, Actinobacillus pleuropneumoniae, Haemophilus, Xylella fastidiosa, Xanthomonas campestris, Xanthomonas axonopodis, Nitrosococcus oceani, Nitrosomonas europaea, Nitrosomonas eutropha, Methylobacillus flagellatus, Thiobacillus denitrificans, Chromobacterium violaceum, Azoarcus, Dechloromonas aromatica, Pseudomonas syringae, Pseudomonas fluorescens, Azotobacter vinelandii, Microbulbifer degradans, Chromohalobacter salexigens, Bordetella pertussis, Bordetella pertussis, Bordetella parapertussis, Bordetella bronchiseptica, Rhodoferax ferrireducens, Rubrivivax gelatinosus, Burkholderia pseudomallei, Burkholderia malle, Burkholderia ambifaria, Burkholderia cenocepacia, Burkholderia vietnamiensis, Burkholderia fungorum, Ralstonia, Ralstonia metallidurans, Ralstonia solanacearum, Brucella melitensis, Brucella abortus, Brucella suis, Brucella, Mesorhizobium, Nitrobacter winogradskyi, Nitrobacter hamburgensis, Rhodopseudomonas palustris, Bartonella henselae, Bartonella Quintana, Sinorhizobium meliloti, Silicibacter, Silicibacter pomeroyi, Rhodobacter sphaeroides, Paracoccus, Paracoccus denitrificans, Jannaschia, Caulobacter cresce, Caulobacter crescentus, Rhodospirillum rubrum, Magnetospirillum magnetotacticum, Erythrobacter litoralis, Sphingopyxis alaskensis, Zymomonas obilis, Gluconobacter oxydans, Trypanosoma cruzi, Trypanosoma brucei, Anabaena variabilis, Nostoc punctiforme, Trichodesmium erythraeum, Crocosphaera watsonii, Synechocystis, Synechococcus elongates, Gloeobacter violaceus, Lactobacillus delbrueckii, Lactobacillus casei, Syphilis spirochete, Treponema pallidum, Chlamydophila pneumoniae, Chlamydophila caviae, Chlamydia trachomatis, Plasmodium falciparum, Plasmodium yoelii, Plasmodium berghei, Cryptosporidium parvum, Methanocaldococcus jannaschii, Methanococcus maripaludis, Aquifex aeolicus, Rickettsia typhi, Rickettsia prowazekii, Rickettsia conorii, Rickettsia rickettsii, Rickettsia felis, Rickettsia akari, Ehrlichia ruminant, Ehrlichia ruminant, Ehrlichia canis, Wolbachia, Legionella pneumophila, Blochmannia floridanus, Blochmannia pennsylvanicus, Prochlorococcus marinus, Synechococcus, Sulfolobus acidocaldarius, Sulfolobus solfataricus, Campylobacter jejuni, Campylobacter coli, Campylobacter upsaliensis, Campylobacter lari, Thiomicrospira denitrificans, Helicobacter pylori, Parachlamydia, Halobacterium, Fusobacterium nucleatum, Solibacter usitatus, Rhodospirillum rubrum, Pelagibacter ubique, Strongylocentrotus purpuratus, Tropheryma whipple, Chloroflexus, Francisella tularensis, Desulfovibrio vulgaris, Acinetobacter, Giardia lamblia, Entamoeba histolytica, and Desulfotalea psychrophila

Preferably, the anti-bacterial agent has an IC₅₀ for the biotin protein ligase of less than 1 μM. More preferably, the anti-bacterial agent has an IC₅₀ for the biotin protein ligase of less than 1 nM.

Such bacterial inhibitors may have also anti-bacterial activity generally, and as such may be used to inhibit the growth of one or more bacteria. The inhibitors may be bacteriocidal or bacteriostatic.

In another form the present invention provides an anti-bacterial agent, the agent inhibiting biotinylation by a bacterial biotin protein ligase.

Preferably, the screening methods for the present invention may be used to identify an inhibitor of a bacterial biotin protein ligase with an IC₅₀ (inhibitory concentration) of less than 3 μM, more preferably with an IC₅₀ of less than 1 μM, more preferably with an IC₅₀ of less than 15 nM, more preferably with an with an IC₅₀ of less than 2 nM, and most preferably with an IC₅₀ of 1 nM or less. In this regard, the screening method for the present invention has been used to identify inhibitors of E. coli biotin protein ligase with an IC₅₀ of less than 1 nM.

The present invention may also be used to identify inhibitors of biotin protein ligases of other pathogenic organisms.

For example, the present invention may be used to identify an inhibitor of a fungal biotin protein ligase.

Accordingly, in another form the present invention provides an inhibitor of a fungal biotin protein ligase.

In another form, the present invention provides an anti-fungal agent, wherein the agent inhibits biotinylation of a substrate by a fungal biotin protein ligase.

Preferably, the anti-fungal agent is an inhibitor of (and inhibits biotinylation of a substrate by) a biotin protein ligase from a fungus selected from the group consisting the genus or species of Aspergillus fumigatus, Aspergillus nidulans, Fusarium graminearinearum, Neurospora crassa, Candida glabrata, Saccharomyces cerevesiae, Kluyveromyces lactis, Eremothecium gossypii, Yarrowia lipolytica, Debaryomyces hansenii, Candida albicans, Schizosaccharomyces pombe, Ustilago maydis, Cryptococcus neoformans.

The present invention may be used to identify an inhibitor of a nematode biotin protein ligase.

Accordingly, in another form the present invention provides an inhibitor of a nematode biotin protein ligase.

In another form, the present invention provides an anti-nematode agent, wherein the agent inhibits biotinylation of a substrate by a nematode biotin protein ligase.

The present invention may be used to identify an inhibitor of a parasite biotin protein ligase.

Accordingly, in another form the present invention provides an inhibitor of a parasite biotin protein ligase.

In another form, the present invention provides an anti-parasitic agent, wherein the agent inhibits biotinylation of a substrate by a nematode biotin protein ligase.

The present invention may be used to identify an inhibitor of an insect biotin protein ligase.

Accordingly, in another form the present invention provides an inhibitor of a insect biotin protein ligase.

In another form, the present invention provides an insecticidal agent, wherein the agent inhibits biotinylation of a substrate by a nematode biotin protein ligase.

The inhibitors may inhibit growth and/or survival of an organism. Accordingly, in another form the present invention provides a method for identifying an inhibitor of growth and/or survival of an organism, the method including the step of identifying an inhibitor of a biotin protein ligase.

In this regard, the screening method for the present invention has been used to identify compounds that inhibit E. coli (IC₅₀=0.9 nM) and S. aureus (IC₅₀=2.7 μM) biotin protein ligases, with the following formula:

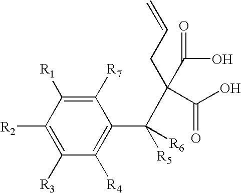

or a salt or ester thereof, wherein R₁ to R₇ are each independently selected from the group consisting of: H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, arylalkyloxy, phenoxy, benzyloxy, heteroaryloxy, amino, alkylamino, aminoalkyl, acylamino, arylamino, sulfonylamino, sulfinylamino, COOH, COR₈, COOR₈, CONHR₈, NHCOR, NHCOOR₈, NHCONHR₈, alkoxycarbonyl, alkylaminocarbonyl, sulfonyl, alkylsulfonyl, alkylsulfinyl, arylsulfonyl, arylsulfinyl, aminosulfonyl, SR₈, R₉S(O)R₁₀—, R₉S(O)₂R₁₀—, R₉C(O)N(R₁₀)R₁₁—, R₉SO₂N(R₁₀)R₁₁—, R₉N(R₁₀)C(O)R₁₁—, R₉N(R₁₀)SO₂R₁₁—, R₉N(R₁₀)C(O)N(R₁₀)R₁₁— and acyl, each of which may be optionally substituted; each R₈, R₉, R₁₀ and R₁₁ is independently selected from the group consisting of a bond, H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

In a preferred embodiment, the compound is 2-allyl-2-(3,5-dimethoxybenzyl)malonic acid, or a salt or ester thereof, as follows:

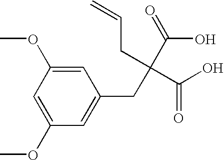

The present invention also provides compositions including an inhibitor of a biotin protein ligase.

The compositions may be used as anti-pathogenic compositions, such as anti-microbial compositions, anti-bacterial compositions, anti-fungal compositions, anti-parasitic compositions, anti-nematode compositions or insecticidal compositions.

In one embodiment, the compositions may be used to inhibit growth and/or survival of an organism, and in particular, for inhibiting the growth and/or survival of pathogenic organisms, such as bacteria and fungi.

Accordingly, in another form the present invention provides a composition for inhibiting growth of a pathogen, the composition including an effective amount of an agent that inhibits a biotin protein ligase.

In a particular preferred form, the compositions include a compound with the following formula:

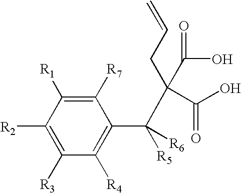

or a salt or ester thereof, wherein R₁ to R₇ are each independently selected from the group consisting of: H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, arylalkyloxy, phenoxy, benzyloxy, heteroaryloxy, amino, alkylamino, aminoalkyl, acylamino, arylamino, sulfonylamino, sulfinylamino, COOH, COR₈, COOR₈, CONHR₈, NHCOR, NHCOOR₈, NHCONHR₈, alkoxycarbonyl, alkylaminocarbonyl, sulfonyl, alkylsulfonyl, alkylsulfinyl, arylsulfonyl, arylsulfinyl, aminosulfonyl, SR₈, R₉S(O)R₁₀—, R₉S(O)₂R₁₀—, R₉C(O)N(R₁₀)R₁₁—, R₉SO₂N(R₁₀)R₁₁—, R₉N(R₁₀)C(O)R₁₁—, R₉N(R₁₀)SO₂R₁₁—, R₉N(R₁₀)C(O)N(R₁₀)R₁₁— and acyl, each of which may be optionally substituted; each R₈, R₉, R₁₀ and R₁₁ is independently selected from the group consisting of a bond, H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

In a preferred embodiment, the compound is 2-allyl-2-(3,5-dimethoxybenzyl)malonic acid, or a salt or ester thereof.

As discussed, the inhibitors of the present invention may also used to inhibit growth and/or survival of an organism, and in particular, the growth and survival of pathogenic organisms of humans, animal or plants.

Accordingly, in another form the present invention provides a method of inhibiting the growth and/or survival of a pathogen organism, the method including the step of contacting a biotin protein ligase of the pathogenic organism with an agent that inhibits biotinylation by the biotin protein ligase.

On one embodiment, the pathogen may be exposed to the agent to inhibit growth and/or survival.

In another form, the present invention provides a method of inhibiting the growth and/or survival of a pathogenic organism, the method including the step of exposing the patheogen to an agent that inhibits biotinylation in the pathogenic organism.

The present invention also provides a composition for inhibiting the growth and/or survival of a pathogenic organism.

In one form, the present invention provides a composition for inhibiting the growth and/or survival of a pathogenic organism, the composition including an effective amount of an agent that inhibits biotinylation in the pathogenic organism.

As discussed previously, the present invention may be used to identify an inhibitor of a biotin protein ligase in one or more cells.

In one embodiment, the present invention may be used to identify an inhibitor of biotinylation in a biological system.

Accordingly, the present invention provides a method for identifying an inhibitor of biotinylation in a biological system, the method including the steps of:
(a) identifying a test compound as an inhibitor of a biotin protein ligase;
(b) determining the ability of the test compound to inhibit biotinylation in a biological system; and
(c) identifying the test compound as an inhibitor of biotinylation in the biological system.

This form of the present invention is directed to the identification of inhibitors of biotinylation by first identifying an inhibitor of a biotin protein ligase. Inhibitors so identified are candidate compounds for inhibiting the growth of pathogenic organisms, and in particular, pathogenic organisms from which the biotin protein ligase was derived.

The biological system in the various forms of the present invention may be any cellular or multi-cellular system for which an inhibition of biotinylation is desired. For example, the biological system may be a cell cultured in vitro, a bacterial cell, a fungal cell, or an entire organism.

Preferably, the biological system includes a cell derived from a pathogenic organism of a human, animal or plant, such as a bacteria, fungus, parasite, nematode or insect. Examples of such pathogenic organisms are as previously hereinbefore described.

Accordingly, in a preferred form the present invention provides a method for identifying an inhibitor of biotinylation in a pathogenic organism of a human, animal or plant, the method including the steps of:
(a) identifying a test compound as an inhibitor of a biotin protein ligase;
(b) determining the ability of the test compound to inhibit biotinylation in a pathogenic organism of a human, animal or plant; and
(c) identifying the test compound as an inhibitor of biotinylation in the pathogenic organism.

Examples of pathogens are as previously herein described. Preferably, the pathogenic organism is a bacterial or fungal pathogen of a human, animal or plant.

The biotin protein ligase may be any suitable biotin protein ligase for which the identification of an inhibitor if biotinylation is required. The biotin protein ligase may be a biotin protein ligase derived from the biological system for which an inhibitor is required, or alternatively, may be a biotin protein ligase from an unrelated biological system. Preferably, the biotin protein ligase is a biotin protein ligase derived from the biological system for which an inhibitor of biotinylation is to be identified. For example, for the identification of an inhibitor of biotinylation in fungi, preferably the biotin ligase is derived from a fungus such as Candida or a yeast.

The identification of a test compound as an inhibitor of a biotin protein ligase may be achieved as previously herein discussed. To determine the ability of the test compound to inhibit biotinylation in the biological system, the test compound will be contacted with the biological system and the extent of biotinylation determined by a suitable method known in the art, including determination by streptavidin blots on enzyme preparations to quantitate the amount of protein-bound biotin and/or enzyme kinetics to determine the specific activity of biotin enzymes.

As will be appreciated, the test compound will be contacted with the biological system in a form and for a time that allows the test compound to be taken up by the biological system. The test compound may be contacted with the biological system in any form or state that allows the test compound to inhibit biotinylation in the biological system. Alternatively, and if feasible, the test compound may be expressed within a cell. An appropriate concentration of the test compound will be selected for determination of the test compound to inhibit biotinylation at that particular concentration.

The identification of the test compound as an inhibitor of a biotinylation in the biological system will be made by a comparison of the extent of biotinylation in the biological system in the presence and absence of the test compound. Preferably, the maximum extent of inhibition of activity by the test compound is greater than 70%. More preferably, the maximum extent of inhibition of activity is greater than 90%.

It has been additionally determined that the present invention may be used to identify compounds that are capable of differentially inhibiting biotin protein ligases from different species. In particular, it has been determined that it is possible to identify compounds that inhibit a biotin protein ligase from a pathogen of a human more effectively than the inhibition of human biotin protein ligase.

As such, these compounds are candidate compounds that may be used to treat an organism for infection by a pathogenic organism.

Accordingly, in another form the present invention provides a method for identifying a compound that differentially inhibits a first biotin protein ligase as compared to inhibition of a second biotin protein ligase, the method including the steps of:
(a) identifying a test compound that inhibits a first biotin protein ligase;
(b) determining the ability of the test compound to inhibit a second biotin protein ligase; and
(c) identifying the test compound as a compound that differentially inhibits the first biotin protein ligase as compared to the inhibition of the second biotin protein ligase.

As will be appreciated, this form of the present invention is directed to the identification of inhibitors that differentially inhibit biotin protein ligases derived from different organisms. Inhibitors so identified are candidate compounds for differentially inhibiting the growth and/or survival of one organism compared to another organism.

In another embodiment, the present invention provides the use of a biotin protein ligase as a target for identifying an agent that differentially inhibits growth of a first organism as compared to growth of a second organism.

In another form, the present invention provides a method for identifying an agent that differentially inhibits growth of a first organism as compared to a second organism, the method including the step of identifying an agent that differentially inhibits a biotin protein ligase from the first organism as compared to a biotin protein ligase from the second organism.

As discussed previously herein, given the high degree of conservation between biotin protein ligases of different species, the ability to identify compounds that differentially inhibit biotin protein ligases of different organisms was contrary to expectation.

Preferably, the differential inhibition is such that the first biotin protein ligase is inhibited and the second biotin protein ligase is not substantially inhibited.

Preferably, the first biotin protein ligase is a biotin protein ligase from an organism that is a pathogen of the organism from which the second biotin protein ligase is derived. More preferably, the first biotin protein ligase is derived from a pathogen of a human, animal or plant, and the second biotin protein ligase is isolated from a host of the pathogen. Examples of pathogenic organisms are as previously hereinbefore described.

Accordingly, in another form the present invention provides a method for identifying a compound that differentially inhibits a biotin protein ligase of a pathogenic organism of a host human, animal or plant, as compared to inhibition of a biotin protein ligase of the host human, animal, or plant, the method including the steps of:
(a) identifying a test compound that inhibits a biotin protein ligase of a pathogenic organism of a host human, animal or plant;
(b) determining the ability of the test compound to inhibit a biotin protein ligase of the host human, animal or plant; and
(c) identifying the test compound as a compound that differentially inhibits the biotin protein ligase of the pathogenic organism as compared to the inhibition of the biotin protein ligase of the host human, animal or plant.

For example, in the case of the second biotin protein ligase being derived from a human or animal, the first biotin protein ligase may be derived from a bacterium, a fungus or a parasite. In the case of the second biotin protein ligase being derived from a plant, the first biotin protein ligase may be derived from a bacterium, a fungus, an insect or a nematode.

Preferably, the first biotin protein ligase is derived from a bacteria or fungus, and the second biotin protein ligase is derived from a human or animal. Most preferably, the first biotin protein ligase is derived from a fungus such a Candida or yeast species, or a bacterium such as E. coli or S. aureus, and the second biotin protein ligase is derived from a human or animal.

The test compound may be identified as a compound capable of differentially inhibiting the first biotin protein ligase as compared to the second biotin protein ligase, by comparing the inhibition of each of the biotin protein ligases in the presence and absence of the test compound. An inhibitor that inhibits the first biotin protein ligase in the presence of the test compound as compared to the absence of the test compound, but which does not inhibit the second biotin protein ligase to the same extent, will be identified as a suitable compound.

Preferably, the test compound inhibits the activity of the first biotin protein ligase by a maximum of 70% or greater. More preferably, the test compound inhibits the activity of the first biotin protein ligase by a maximum of 90% or greater.

Preferably, the inhibitor does not inhibit the activity of a second biotin protein ligase by more than 10%. More preferably, the inhibitor does not inhibit the activity of the second biotin protein ligase by more than 5%.

The present invention also provides a differential inhibitor identified by the relevant methods for the present invention. Accordingly, the present invention also provides an agent that differentially inhibits a first biotin protein as compared to a second biotin protein ligase.

The differential inhibitor may inhibit a biotin protein ligase of a pathogen of a human, animal or plant, and not substantively inhibit a human, animal or plant biotin protein ligase. Accordingly, in another form the present invention provides an inhibitor of a biotin protein ligase of a pathogen of a human, animal or plant, wherein the inhibitor does not substantively inhibit a human, plant or animal biotin protein ligase.

Examples of pathogenic organisms are as previously hereinbefore described.

For example, the present invention may be used to identify a compound that inhibits a bacterial biotin protein ligase and which does not substantively inhibit a human, plant or animal biotin protein ligase. Accordingly, in another form the present invention provides an inhibitor of a bacterial biotin protein ligase, wherein the inhibitor does not substantively inhibit a human, plant or animal biotin protein ligase.

In this regard, the screening method for the present invention has been used to identify a compound that inhibits E. coli (IC₅₀=0.9 nM) and S. aureus (IC₅₀=2.7 μM) biotin protein ligases, but which does not substantively inhibit human biotin protein ligase (IC₅₀>50 μM), with the following formula:

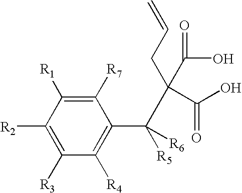

or a salt or ester thereof, wherein R₁ to R₇ are each independently selected from the group consisting of: H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, arylalkyloxy, phenoxy, benzyloxy, heteroaryloxy, amino, alkylamino, aminoalkyl, acylamino, arylamino, sulfonylamino, sulfinylamino, COOH, COR₈, COOR₈, CONHR₈, NHCOR, NHCOOR₈, NHCONHR₈, alkoxycarbonyl, alkylaminocarbonyl, sulfonyl, alkylsulfonyl, alkylsulfinyl, arylsulfonyl, arylsulfinyl, aminosulfonyl, SR₈, R₉S(O)R₁₀—, R₉S(O)₂R₁₀—, R₉C(O)N(R₁₀)R₁₁—, R₉SO₂N(R₁₀)R₁₁—, R₉N(R₁₀)C(O)R₁₁—, R₉N(R₁₀)SO₂R₁₁—, R₉N(R₁₀)C(O)N(R₁₀)R₁₁— and acyl, each of which may be optionally substituted; each R₈, R₉, R₁₀ and R₁₁ is independently selected from the group consisting of a bond, H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

In a preferred embodiment, the compound is 2-allyl-2-(3,5-dimethoxybenzyl)malonic acid, or a salt thereof.

The present invention may be used to identify a compound that inhibits a fungal biotin protein ligase and which does not substantively inhibit a human, plant or animal biotin protein ligase. Accordingly, in another form the present invention provides an inhibitor of a fungal biotin protein ligase, wherein the inhibitor does not substantively inhibit a human, plant or animal biotin protein ligase.

The present invention also provides a method for identifying a compound that differentially inhibits biotinylation in a first biological system as compared to inhibition of biotinylation in a second biological system, the method including the steps of:
(a) identifying a test compound that inhibits biotinylation in a first biological system;
(b) determining the ability of the test compound to inhibit biotinylation in a second biological system; and
(c) identifying the test compound as a compound that differentially inhibits biotinylation in the first biological system as compared to the inhibition of biotinylation in the second biological system.

As will be appreciated, this form of the present invention is directed to the identification of inhibitors that differentially inhibit biotinylation in a first biological system as compared to a second biological system. Inhibitors so identified are candidate compounds for differentially inhibiting the growth of one organism compared to another organism.

Preferably, the differential inhibition is such that the biotinylation in the first biological system is inhibited and biotinylation in the second biological system is not substantially inhibited.

Preferably, the first biological system is a biological system derived from an organism that is a pathogen of the organism from which the second biological system is derived. More preferably, the first biological system is derived from a pathogen of a human, animal or plant, and the second biological system is isolated from the host of the pathogen of the first biological system.

Accordingly, in another form the present invention provides a method for identifying a compound that differentially inhibits biotinylation in a pathogenic organism of a host human, animal or plant, as compared to inhibition of biotinylation in the host human, animal or plant, the method including the steps of:
(a) identifying a test compound that inhibits biotinylation in a pathogenic organism of a host human, animal or plant;
(b) determining the ability of the test compound to inhibit biotinylation in the host human, animal or plant; and
(c) identifying the test compound as a compound that differentially inhibits biotinylation in the pathogenic organism as compared to the inhibition of biotinylation in the host human, animal or plant.

For example, in the case of the second biological system being derived from a human or animal, the first biological system may be derived from a bacteria, fungus or parasite. In the case of the second biological system being derived from a plant, the first biological system may be derived from a bacteria, fungus, insect or nematode.

More preferably, the first biological system is derived from a bacteria or a fungus, and the second biological system is derived from a human or animal.

The test compound may be identified as a compound capable of differentially inhibiting biotinylation in the first biological system as compared to the second biological system, by methods as previously described.

The test compound may be identified as a compound capable of differentially inhibiting biotinylation in a first biological system as compared to a second biological system, by comparing the inhibition of biotinylation for each of the biological systems in the presence and absence of the test compound. A compound that inhibits the biotinylation in the first biological system in the presence of the test compound as compared to the absence of the test compound, and which does not inhibit biotinylation in a second biological system to the same extent, will be identified as a suitable compound.

Preferably, the test compound inhibits biotinylation in the first biological system by 70% or greater. More preferably, the test compound inhibits biotinylation in the first biological system by 90% or greater.

Preferably, the test compound does not inhibit biotinylation in the second biological system by more than 10%. More preferably, the test compound does not inhibit biotinylation in the second biological system by more than 5%.

Differential inhibitors identified by the various methods for the present invention may be used to prevent and/or treat the growth of a pathogenic organism in a subject.

For example, the differential inhibitors may be used to prevent and/or treat a human, animal or plant subject having a local and/or systemic infection by a pathogenic organism, by inhibiting the activity of a biotin protein ligase of the pathogenic organism.

Accordingly, in another form the present invention provides a method for preventing and/or treating an infection by a pathogenic organism of a subject, the method including the step of administering to the subject an effective amount of an agent that inhibits a biotin protein ligase of the pathogenic organism.

This form of the present invention results in biotinylation in the pathogenic organism being inhibited, thus resulting in an inhibition of growth and/or survival of the pathogenic organism in the subject.

Accordingly, in another the present invention provides a method for preventing and/or treating an infection by a pathogenic organism of a subject, the method including the step of administering to the subject an effective amount of an agent that inhibits biotinylation in the pathogenic organism.

Examples of agents include drugs, small molecules, nucleic acids, oligonucleotides, peptides, polypeptides, proteins, enzymes, polysaccharides, glycoproteins, lipids, antibodies or a part thereof, and aptamers.

In particular preferred form, the agent administered is a compound with the following chemical formula:

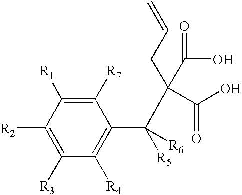

or an acceptable salt or ester thereof, wherein R₁ to R₇ are each independently selected from the group consisting of: H, halogen, alkyl, alkenyl, alkynyl, haloalkyl, haloalkenyl, heteroalkyl, cycloalkyl, cycloalkenyl, heterocycloalkyl, heterocycloalkenyl, aryl, heteroaryl, cycloalkylalkyl, heterocycloalkylalkyl, arylalkyl, heteroarylalkyl, arylalkenyl, cycloalkylheteroalkyl, heterocycloalkylheteroalkyl, heteroarylheteroalkyl, arylheteroalkyl, hydroxy, hydroxyalkyl, alkoxy, alkoxyalkyl, alkoxyaryl, alkenyloxy, alkynyloxy, cycloalkylkoxy, heterocycloalkyloxy, aryloxy, arylalkyloxy, phenoxy, benzyloxy, heteroaryloxy, amino, alkylamino, aminoalkyl, acylamino, arylamino, sulfonylamino, sulfinylamino, COOH, COR₈, COOR₈, CONHR₈, NHCOR, NHCOOR₈, NHCONHR₈, alkoxycarbonyl, alkylaminocarbonyl, sulfonyl, alkylsulfonyl, alkylsulfinyl, arylsulfonyl, arylsulfinyl, aminosulfonyl, SR₈, R₉S(O)R₁₀—, R₉S(O)₂R₁₀—, R₉C(O)N(R₁₀)R₁₁—, R₉SO₂N(R₁₀)R₁₁—, R₉N(R₁₀)C(O)R₁₁—, R₉N(R₁₀)SO₂R₁₁—, R₉N(R₁₀)C(O)N(R₁₀)R₁₁— and acyl, each of which may be optionally substituted; each R₈, R₉, R₁₀ and R₁₁ is independently selected from the group consisting of a bond, H, alkyl, alkenyl, alkynyl, haloalkyl, heteroalkyl, cycloalkyl, heterocycloalkyl, aryl, heteroaryl, cycloalkylalkyl, heterocycloalkylalkyl, arylalkyl, heteroarylalkyl and acyl, each of which may be optionally substituted.

In a preferred embodiment, the compound is 2-allyl-2-(3,5-dimethoxybenzyl)malonic acid, or an acceptable salt thereof.

This compound may be synthesized as described in Zhao, Ya Jun; Tang, Hui Tong; Zhang, Pang (1997) “A study on cycloacylation related to synthesis of perylenequinones.” Chinese Chemical Letters 8(9): 769-772.

In another form, the present invention provides a composition including the above agents for the prevention and/or treatment of an infection of a host by a pathogenic organism, and the use of such agents for the preparation of a medicament for preventing and/or treating an infection of a host by a pathogenic organism.

In one form the present invention provides a composition for preventing and/or treating method an infection of a host by a pathogenic organism of a subject, the composition including an effective amount of an agent that inhibits a biotin protein ligase of the pathogenic organism.

In another form the present invention provides a composition for preventing and/or treating an infection of a host by a pathogenic organism, the composition including an effective amount of an agent that inhibits biotinylation in the pathogenic organism.

In the case of administration of an agent to a human or animal subject (ie host), the agent may be administered to the subject in a suitable form to inhibit the activity of the biotin protein ligase in the pathogenic organism. The effective amount of agent to be administered is not particularly limited, so long as it is within such an amount and in such a form that generally exhibits a useful or therapeutic effect.

In this regard, an effective amount of the agent may be appropriately chosen, depending upon, for example, the type and extent of infection by the pathogenic organism, the age and body weight of the subject, the frequency of administration, and the presence of other active agents.

The administration of the agent may be within any time suitable to produce the desired effect of preventing and/or treating infection by the pathogenic organism.

The agent may be administered orally, parenterally, topically or by any other suitable means, and therefore transit time of the agent must be taken into account.

The administration of the agent may also include the use of one or more pharmaceutically acceptable additives, including pharmaceutically acceptable salts, amino acids, polypeptides, polymers, solvents, buffers, excipients and bulking agents, taking into consideration the particular physical and chemical characteristics of the agent to be administered.

For example, the agent can be prepared into a variety of pharmaceutical compositions in the form of, e.g., an aqueous solution, an oily preparation, a fatty emulsion, an emulsion, a gel, etc., and these preparations can be administered as intramuscular or subcutaneous injection or as injection to an organ, or as an embedded preparation or as a transmucosal preparation through nasal cavity, rectum, uterus, vagina, lung, etc. The composition may be administered in the form of oral preparations (for example solid preparations such as tablets, capsules, granules or powders; liquid preparations such as syrup, emulsions or suspensions). Compositions containing the agent may also contain a preservative, stabiliser, dispersing agent, pH controller or isotonic agent. Examples of suitable preservatives are glycerin, propylene glycol, phenol or benzyl alcohol. Examples of suitable stabilisers are dextran, gelatin, a-tocopherol acetate or alpha-thioglycerin. Examples of suitable dispersing agents include polyoxyethylene (20), sorbitan mono-oleate (Tween 80), sorbitan sesquioleate (Span 30), polyoxyethylene (160) polyoxypropylene (30) glycol (Pluronic F68) or polyoxyethylene hydrogenated castor oil 60. Examples of suitable pH controllers include hydrochloric acid, sodium hydroxide and the like. Examples of suitable isotonic agents are glucose, D-sorbitol or D-mannitol.

The administration of the agent in the various forms of the present invention may also be in the form of a composition containing a pharmaceutically acceptable carrier, diluent, excipient, suspending agent, lubricating agent, adjuvant, vehicle, delivery system, emulsifier, disintegrant, absorbent, preservative, surfactant, colorant, flavorant or sweetener, taking into account the physical and chemical properties of the agent being administered.

For these purposes, the composition may be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term parenteral as used herein includes subcutaneous, intravenous, intramuscular, intraperitoneal, intrathecal, intraventricular, intrasternal, and intracranial injection or infusion techniques.

When administered parenterally, the composition will normally be in a unit dosage, sterile injectable form (solution, suspension or emulsion) which is preferably isotonic with the blood of the recipient with a pharmaceutically acceptable carrier. Examples of such sterile injectable forms are sterile injectable aqueous or oleaginous suspensions. These suspensions may be formulated according to techniques known in the art using suitable dispersing or wetting agents and suspending agents. The sterile injectable forms may also be sterile injectable solutions or suspensions in non-toxic parenterally-acceptable diluents or solvents, for example, as solutions in 1,3-butanediol. Among the acceptable vehicles and solvents that may be employed are water, saline, Ringer's solution, dextrose solution, isotonic sodium chloride solution, and Hanks' solution. In addition, sterile, fixed oils are conventionally employed as solvents or suspending mediums. For this purpose, any bland fixed oil may be employed including synthetic mono- or di-glycerides, corn, cottonseed, peanut, and sesame oil. Fatty acids such as ethyl oleate, isopropyl myristate, and oleic acid and its glyceride derivatives, including olive oil and castor oil, especially in their polyoxyethylated versions, are useful in the preparation of injectables. These oil solutions or suspensions may also contain long-chain alcohol diluents or dispersants.

The carrier may contain minor amounts of additives, such as substances that enhance solubility, isotonicity, and chemical stability, for example anti-oxidants, buffers and preservatives.

When administered orally, the agent will usually be formulated into unit dosage forms such as tablets, cachets, powder, granules, beads, chewable lozenges, capsules, liquids, aqueous suspensions or solutions, or similar dosage forms, using conventional equipment and techniques known in the art. Such formulations typically include a solid, semisolid, or liquid carrier. Exemplary carriers include lactose, dextrose, sucrose, sorbitol, mannitol, starches, gum acacia, calcium phosphate, mineral oil, cocoa butter, oil of theobroma, alginates, tragacanth, gelatin, syrup, methyl cellulose, polyoxyethylene sorbitan monolaurate, methyl hydroxybenzoate, propyl hydroxybenzoate, talc, magnesium stearate, and the like.

A tablet may be made by compressing or molding the agent optionally with one or more accessory ingredients. Compressed tablets may be prepared by compressing, in a suitable machine, the active ingredient in a free-flowing form such as a powder or granules, optionally mixed with a binder, lubricant, inert diluent, surface active, or dispersing agent. Moulded tablets may be made by moulding in a suitable machine, a mixture of the powdered active ingredient and a suitable carrier moistened with an inert liquid diluent.

The administration of the agent may also utilize controlled release technology. The agent may also be administered as a sustained-release pharmaceutical. To further increase the sustained release effect, the agent may be formulated with additional components such as vegetable oil (for example soybean oil, sesame oil, camellia oil, castor oil, peanut oil, rape seed oil); middle fatty acid triglycerides; fatty acid esters such as ethyl oleate; polysiloxane derivatives; alternatively, water-soluble high molecular weight compounds such as hyaluronic acid or salts thereof (weight average molecular weight: ca. 80,000 to 2,000,000), carboxymethylcellulose sodium (weight average molecular weight: ca. 20,000 to 400,000), hydroxypropylcellulose (viscosity in 2% aqueous solution: 3 to 4,000 cps), atherocollagen (weight average molecular weight: ca. 300,000), polyethylene glycol (weight average molecular weight: ca. 400 to 20,000), polyethylene oxide (weight average molecular weight: ca. 100,000 to 9,000,000), hydroxypropylmethylcellulose (viscosity in 1% aqueous solution: 4 to 100,000 cSt), methylcellulose (viscosity in 2% aqueous solution: 15 to 8,000 cSt), polyvinyl alcohol (viscosity: 2 to 100 cSt), polyvinylpyrrolidone (weight average molecular weight: 25,000 to 1,200,000).

Alternatively, the agent may be incorporated into a hydrophobic polymer matrix for controlled release over a period of days. The agent may then be moulded into a solid implant, or externally applied patch, suitable for providing efficacious concentrations of the agent over a prolonged period of time without die need for frequent re-dosing. Such controlled release films are well known to the art. Other examples of polymers commonly employed for this purpose that may be used include nondegradable ethylene-vinyl acetate copolymer a degradable lactic acid-glycolic acid copolymers which may be used externally or internally. Certain hydrogels such as poly(hydroxyethylmethacrylate) or poly(vinylalcohol) also may be useful, but for shorter release cycles than the other polymer release systems, such as those mentioned above.

The carrier may also be a solid biodegradable polymer or mixture of biodegradable polymers with appropriate time release characteristics and release kinetics. The agent may then be moulded into a solid implant suitable for providing efficacious concentrations of the agent over a prolonged period of time without the need for frequent re-dosing. The agent can be incorporated into the biodegradable polymer or polymer mixture in any suitable manner known to one of ordinary skill in the art and may form a homogeneous matrix with the biodegradable polymer, or may be encapsulated in some way within the polymer, or may be moulded into a solid implant.

For topical administration, the composition of the present invention may be in the form of a solution, spray, lotion, cream (for example a non-ionic cream), gel, paste or ointment. Alternatively, the composition may be delivered via a liposome, nanosome, rivosome, or nutri-diffuser vehicle. Topical administration is particularly suitable for local infections by a pathogenic organism.

A cream is a formulation that contains water and oil and is stabilized with an emulsifier. Lipophilic creams are called water-in-oil emulsions, and hydrophilic creams oil-in-water emulsions. The cream base for water-in-oil emulsions are normally absorption bases such as vaseline, ceresin or lanolin. The bases for oil-in-water emulsions are mono-, di- and triglycerides of fatty acids or fatty alcohols with soaps, alkyl sulphates or alkyl polyglycol ethers as emulsifiers.

A lotion is an opaque, thin, non-greasy emulsion liquid dosage form for external application to the skin, which generally contains a water-based vehicle with greater than 50% of volatiles and sufficiently low viscosity that it may be delivered by pouring.

Lotions are usually hydrophilic, and contain greater than 50% of volatiles as measured by LOD (loss on drying). A lotion tends to evaporate rapidly with a cooling sensation when rubbed onto the skin.

A paste is an opaque or translucent, viscous, greasy emulsion or suspension semisolid dosage form for external application to the skin, which generally contains greater than 50% of hydrocarbon-based or a polyethylene glycol-based vehicle and less than 20% of volatiles. A paste contains a large proportion (20-50%) of dispersed solids in a fatty or aqueous vehicle. An ointment tends not to evaporate or be absorbed when rubbed onto die skin.

An ointment is an opaque or translucent, viscous, greasy emulsion or suspension semisolid dosage form for external application to the skin, which generally contains greater than 50% of hydrocarbon-based or a polyethylene glycol-based vehicle and less than 20% of volatiles. An ointment is usually lipophilic, and contains >50% of hydrocarbons or polyethylene glycols as the vehicle and <20% of volatiles as measured by LOD. An ointment tends not to evaporate or be absorbed when rubbed onto the skin.

A gel is usually a translucent, non-greasy emulsion or suspension semisolid dosage form for external application to the skin, which contains a gelling agent in quantities sufficient to impart a three-dimensional, cross-linked matrix. A gel is usually hydrophilic, and contains sufficient quantities of a gelling agent such as starch, cellulose derivatives, carbomers, magnesium-aluminium silicates, xanthan gum, colloidal silica, aluminium or zinc soaps.

The composition for topical administration may further include drying agents, anti-foaming agents; buffers, neutralizing agents, agents to adjust pH; colouring agents and decolouring agents; emollients; emulsifying agents, emulsion stabilizers and viscosity builders; humectants; odorants; preservatives, antioxidants, and chemical stabilizers; solvents; and thickening, stiffening, and suspending agents, and a balance of water or solvent.

It should also be appreciated that other methods for delivery of an agent are contemplated. For example, the agent may be delivered by way of a nucleic acid or vector that allows for expression of the agent in the appropriate target cells. For example, the agent may be delivered by way of a viral vector that causes expression of the agent in target cells.

Methods are also known in the art for administering an agent to a plant to prevent and/or treat an infection by a pathogenic organism. For example, the plant may be treated for a systemic infection by a pathogen, a local infection by a pathogen, or seeds may be treated to remove surface pathogens. As will be appreciated by a person skilled in the art, the formulation of the agent will vary depending upon the particular treatment or prevention regime adopted.

The present invention also provides an isolated nucleic acid sequence encoding a biotin protein ligase or a fragment thereof, wherein the nucleic acid includes at least one codon that is modified from the wild type nucleic acid sequence encoding the biotin protein ligase or fragment thereof.

Preferably, the isolated nucleic acid encodes a biotin protein ligase (or a part thereof) from a biotin protein ligase of a pathogenic organism of a human, plant or animal, such as a bacteria, fungi, parasite, insect or nematode. More preferably, the isolated nucleic acid sequence encodes a biotin protein ligase from a fungus. More preferably, the nucleic acid encodes a biotin protein ligase from a Candida species. Most preferably, the nucleic acid encodes a biotin protein ligase from Candida albicans.

Modifications to the at least one codon include modifications to change the identity of the amino acid encoded by the codon, or modifications to change the sequence of a codon encoding the same amino acid.

In this regard, it has been found that the expression and activity of biotin protein ligase expressed in bacteria from a nucleic acid sequence encoding a biotin protein ligase from Candida may be enhanced by modifying a CUG codon (which normally encodes a leucine amino acid) to a codon specifying a serine amino acid (ie UCU, UCC, UCG, UCA, AGU, AGC).

Accordingly, in a preferred form the present invention provides an isolated nucleic acid encoding a Candida biotin protein ligase or a fragment thereof, wherein the nucleic acid includes at least one codon specifying a serine amino acid serine as a replacement for a CUG codon in the corresponding position of the wildtype sequence of the biotin protein ligase.

Preferably the codon specifying the amino acid serine is an AGU codon.

The modifications to the at least one codon may be made by a suitable method known in the art, including the use of site-directed mutagenesis, PCR using primers having the desired mutation or mutations incorporated therein, or by random mutagenesis, such methods as essentially described in Sambrook, J., Fritsch, E. F., and Maniatis, T., in Molecular Cloning: A Laboratory Manual. Cold Spring Harbor Laboratory Press, NY, Vol. 1, 2, 3 (1989). The nucleic acid sequence may be isolated by a suitable method known in the art. The isolated nucleic acid may encode an entire biotin protein ligase containing at least one codon that is modified from the wild type nucleic acid sequence, or part of a biotin protein ligase containing at least one codon that is modified from the wild type nucleic acid sequence.

The isolated nucleic acid may be further cloned into a plasmid for expression of the cloned biotin protein ligase. The plasmid encoding the cloned biotin protein ligase may then be transformed or transfected into a prokaryotic or eukaryotic cell for expression of the cloned biotin protein ligase.

The present invention also provides an isolated polypeptide fragment of a human or Candida pyruvate carboxylase, wherein the fragment is capable of biotinylation by a biotin protein ligase.

In this regard, it has been found that isolated polypeptide fragments of a human or Candida pyruvate carboxylase may be produced that still retain the capacity to be biotinylated by a biotin protein ligase.

In the case of an isolated polypeptide fragment of human pyruvate carboxylase, preferably the isolated polypeptide fragment of human pyruvate carboxylase includes (or consists of) the following amino acid sequence (or a functional variant thereof):

(SEQ ID No.9)

M¹⁰⁹⁸HFHPKALKDVKGQIGAPMPGKVIDIKVVAGAKVAKGQPLCVLSAM
KMETVVTSPMEGTVRKVHVTKDMTLEGDDLILEIE¹¹⁷⁸

Most preferably, the isolated polypeptide fragment of human pyruvate carboxylase includes (or consists of) one of the following amino acid sequences (or a functional variant thereof):

hPC-108:

(SEQ ID No.10)

A¹⁰⁷¹GQRQVFFELNGQLRSILVKDTQAMKEMHFHPKALKDVKGQIGAPM
PGKVIDIKVVAGAKVAKGQPLCVLSAMKMETVVTSPMEGTVRKVHVTKDM
TLEGDDLILEIE¹¹⁷⁸
hPC-81:

(SEQ ID No.11)

M¹⁰⁹⁸HFHPKALKDVKGQIGAPMPGKVIDIKVVAGAKVAKGQPLCVLSAM
KMETVVTSPMEGTVRKVHVTKDMTLEGDDLILEIE¹¹⁷⁸

In the case of an isolated polypeptide fragment of a Candida pyruvate carboxylase, preferably the polypeptide fragment is from Candida albicans. Most preferably, the isolated polypeptide fragment includes (or consists of) the following amino acid sequence (or a functional variant thereof):

(SEQ ID No.12)

K⁹⁰⁹ASASNEVGAPMAGVVIEIRAHKHQQIAKGDPIAVLSAMKMEMVISA
PCSGEIGDILIHEGDSVDANDLITSIH⁹⁸²

More preferably, the isolated polypeptide fragment of Candida pyruvate carboxylase includes (or consists of) of one of the following amino acid sequences (or a functionally variant thereof):

caPC-115:

(SEQ ID No.13)

M⁸⁶⁸AVGDVSEKTGTREVFFELNGEMRSVSVEDKTVSVELKTRPKASASN
EVGAPMAGVVIEIRAHKHQQIAKGDPIAVLSAMKMEMVISAPCSGEIGDI
LIHEGDSVDANDLITSIH⁹⁸²
caPC-93:

(SEQ ID No.14)

M⁸⁹⁰RSVSVEDKTVSVELKTRPKASASNEVGAPMAGVVIEIRAHKHQQIA
KGDPIAVLSAMKMEMVISAPCSGEIGDILIHEGDSVDANDLITSIH⁹⁸²
caPC-74:

(SEQ ID No.15)

K⁹⁰⁹ASASNEVGAPMAGVVIEIRAHKHQQIAKGDPIAVLSAMKMEMVISA
PCSGEIGDILIHEGDSVDANDLITSIH⁹⁸²

The polypeptides may be produced by methods known in the art using chemical synthesis or by expression and purification of the appropriate cloned nucleotide sequence in an appropriate expression system. In the case of expression and purification of the polypeptides, preferably the polypeptides are fused to a moiety to facilitate purification of the polypeptides, such as the fusion of the polypeptides with GST or the addition of a six histidine tag.

Confirmation of the ability of an isolated polypeptide to be biotinylated by a biotin protein ligase may be determined by a suitable method. For example, the isolated polypeptide may be used as a substrate in an in vitro reaction with biotin and biotin protein ligase, and the extent of biotinylation of the isolated polypeptide determined. Alternatively, the polypeptide may be expressed in a cell and the extent of biotinylation by the endogenous biotin protein ligase (or a cloned biotin protein ligase expressed in the cell) may be determined by a suitable method known in the art.

Substrates that are efficiently biotinylated by biotin protein ligase, as compared to the biotinylation of a known substrate of a biotin protein ligase, may then be identified.

An example of a suitable reaction mix for determination of the ability of an isolated polypeptide to be biotinylated in vitro is as follows:

The isolated polypeptide may be adsorbed onto wells (eg Griener Lumitrac 600 White 96-well plates; Stennick Scientific) by coating overnight at 4° C. in 100 uL of Tris Buffered saline (TBS, pH 7.5). Wells may then be blocked in 1% BSA solution in TBS for 1 hr at 37° C., the blocking buffer removed and wells washed in TBS buffer containing 0.1% Tween-20. BPL reaction mix may then be added to each well. The BPL reaction mix is 50 mM Tris-HCl pH 8.0, 100 uM ATP, 5.5 mM MgCl₂, 0.1 mg/ml BSA, 10 uM biotin, 0.1 uM dithiothreitol. BPL reactions are initiated by the addition of enzyme to a final concentration of 1-5 nM. The reaction is then allowed to proceed at 37° C. for 15 minutes at which point it is terminated by the addition of 5 ul 0.5 M EDTA (final concentration 25 mM). The reaction mix may then be discarded and wells washed in TBS-TD (TBS, 0.1% Tween-20 and 100 uM diethylenetriaminepentaacetic acid (DPTA)). The quantitative analysis of biotinylated protein formed in the reaction and coupled onto the well surface may then be determined using time resolved fluorescence. Europium labelled streptavidin (Perkin-Elmer) solution, diluted to 0.1 ug/ml in TBS-TD, is added to each well. The plates may then be incubated at 37° C. for 1 hr before being washed in TBS-TD and in water. DELFIA Enhancement solution (Perkin Elmer) may then be added and incubated for ten minutes at room temperature before quantitation.

### DESCRIPTION OF THE PREFERRED EMBODIMENTS

Reference will now be made to experiments that embody the above general principles of the present invention. However, it is to be understood that the following description is not to limit the generality of the above description.

### Example 1

### Cloning the Biotin Domains of Candida albicans and Human Pyruvate Carboxylase

Biotin protein ligases recognise a structured biotin domain and covalently modify a specific target lysine in the folded substrate. The ability of a number of peptides to function as a substrate for the endogenous biotin protein ligase in E. coli was determined using an in vivo biotinylation assay.

Two polypeptides (hPC-108 and hPC-81) containing the biotin domain from human pyruvate carboxylase were used. Three polypeptides (caPC-115, caPC-93 and caPC-74) from Candida albicans pyruvate carboxylase were also used.

Peptides encompassing predicted biotin domains of human and C. albicans pyruvate carboxylase were expressed as fusions to GST to facilitate high-level expression of each sequence and the quantitation of protein yields by Western blot. Biotinylation of the substrates was determined by Western-blot using the biotin-binding protein streptavidin as a probe.

The amino acid sequence for each of the human and Candida peptides are as follows (amino acid positions indicated are the relative position of the corresponding amino acid in the sequence of the full-length protein):

hPC-108:

(SEQ ID No. 16)

A¹⁰⁷¹GQRQVFFELNGQLRSILVKDTQAMKEMHFHPKALKDVKGQIGAPM
PGKVIDIKVVAGAKVAKGQPLCVLSAMKMETVVTSPMEGTVRKVHVTKDM
TLEGDDLILEIE¹¹⁷⁸
hPC-81:

(SEQ ID No. 17)

M¹⁰⁹⁸HFHPKALKDVKGQIGAPMPGKVIDIKVVAGAKVAKGQPLCVLSAM
KMETVVTSPMEGTVRKVHVTKDMTLEGDDLILEIE¹¹⁷⁸
caPC-115:

(SEQ ID No. 18)

M⁸⁶⁸AVGDVSEKTGTREVFFELNGEMRSVSVEDKTVSVELKTRPKASASN
EVGAPMAGVVIEIRAHKHQQIAKGDPIAVLSAMKMEMVISAPCSGEIGDI
LIHEGDSVDANDLITSIH⁹⁸²
caPC-93:

(SEQ ID No. 19)

M⁸⁹⁰RSVSVEDKTVSVELKTRPKASASNEVGAPMAGVVIEIRAHKHQQIA
KGDPIAVLSAMKMEMVISAPCSGEIGDILIHEGDSVDANDLITSIH⁹⁸²
caPC-74:

(SEQ ID No. 20)

K⁹⁰⁹ASASNEVGAPMAGVVIEIRAHKHQQIAKGDPIAVLSAMKMEMVISA
PCSGEIGDILIHEGDSVDANDLITSIH⁹⁸²

To construct plasmids suitable for expressing the C-terminal 74, 93 and 115 amino acids of C. albicans pyruvate carboxylase, the DNA encoding each peptide was amplified using PCR with genomic DNA. Sequence data for Candida albicans pyruvate carboxylase was obtained from the Stanford Genome Technology Centre (orf6.2989 orf6-2189:1646-4594).

To amplify DNA for the 74 amino acid peptide (caPC-74) from Candida albicans pyruvate carboxylase, the following oligonucleotides were used:

C18/38

(SEQ ID No. 21)

[5′ATCTACGGATCCAAAGCTTCAGCATCAAATGAAGTTGG];
C19/43

(SEQ ID No. 22)

[5′ATCTACGAATTCATCAATGAATACTAGTAATCAAATCATTAGC]

To amplify DNA for the 93 amino acid peptide (caPC-93) from Candida albicans pyruvate carboxylase, the following oligonucleotides were used:

C17/34

(SEQ ID No. 23)

[5′ATCTACGGATCCATGAGATCAGTTTCCGTTGAAG];
C19/43

(SEQ ID No. 24)

[5′ATCTACGAATTCATCAATGAATACTAGTAATCAAATCATTAGC]

To amplify DNA for the 115 amino acid peptide (caPC-115) from Candida albicans pyruvate carboxylase, the following oligonucleotides were used:

C20/28

(SEQ ID No. 25)

[5′GGATCCATGGCTGTTGGTGATGTTTCGG];
C19/43

(SEQ ID No. 26)

[5′ATCTACGAATTCATCAATGAATACTAGTAATCAAATCATTAGC]

To amplify the above DNAs, PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers were included along with 1 ug of genomic DNA as the template. Thermocycling conditions were 30 cycles of 92° C. for 30 seconds, 60° C. for 30 seconds and 72° C. for 45 seconds.

The above oligonucleotides introduced a BamH1 restriction site at the 5′ end of the product and an EcoR1 site at the 3′ end. The PCR products were digested with BamH1 and EcoR1 restriction endonucleases and ligated into similarly treated pGEX-4T-2 plasmid (Amersham-Biosciences).

To construct plasmids suitable for expressing the C-terminal 81 and 108 amino acids of human pyruvate carboxylase, the DNA encoding each peptide was amplified using PCR with genomic DNA. The sequence data required for cloning human pyruvate carboxylase was obtained from GenBank accession number U30891.

To construct expression vectors suitable for expressing the C-terminal 81 and 108 amino acids of human pyruvate carboxylase, the DNA encoding each peptide was amplified using PCR from plasmid pEF-PC, which contains the full-length cDNA encoding human pyruvate carboxylase, as described in Hobbs et al. (1998) Biochem Biophys Res Commun. 252(2):368-272.

To amplify DNA for the 81 amino acid peptide (hPC-81) from human pyruvate carboxylase, the following oligonucleotides were used:

81F

(SEQ ID No. 27)

[5′AATTGGTAGAATTCATGCACTTCCACCCCAAGGCC];
Pcrev

(SEQ ID No. 28)

[5′GATTAATTCTCGAGTTATCACTCGATCTCCAGGATGAGGTCG]

To amplify DNA for the 108 amino acid peptide (hPC-81) from human pyruvate carboxylase, the following oligonucleotides were used:

108F

(SEQ ID No. 29)

[5′AATTGGTAGAATTCGCCGGCCAGAGGCAGGTCTTC];
Pcrev

(SEQ ID No. 30)

[5′GATTAATTCTCGAGTTATCACTCGATCTCCAGGATGAGGTCG]

To amplify the above DNAs, PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA.

Between 0.1 and 0.5 uM of the required oligonucleotide primers were included along with either 1 ng of pEF-PC plasmid containing the target sequence. Thermocycling conditions were 25 cycles of 92° C. for 30 seconds, 60° C. for 30 seconds and 72° C. for 45 seconds.

The above oligonucleotides introduced an EcoR1 restriction site at the 5′ end of the product and a Xho1 site at the 3′ end. The PCR products were digested with EcoR1 and Xho1 restriction endonucleases and ligated into similarly treated pGEX-4T-2, essentially as described in Sambrook, J., Fritsch, E. F., and Maniatis, T., in Molecular Cloning: A Laboratory Manual. Cold Spring Harbor Laboratory Press, NY, Vol. 1, 2, 3 (1989).

### Example 2

### Analysis of Peptide Fragments from C. albicans and Human Pyruvate Carboxylase as Biotin Protein Ligase Substrates

The ability of each of the Candida and human peptides to function as a substrate for biotin protein ligase in vivo was performed in E. coli. Each pGEX-4T-2 derived construct was transformed into E. coli BL21 cells by CaCl₂ mediated transformation and colonies grown in 2YT media until log phase. Expression of the GST fusion proteins was induced with 0.1 mM IPTG for 1 hour at 37° C. before cells were harvested.

Biotinylation of the fusion proteins was assayed on whole cell lysates by streptavidin-blot. Whole cell lysates were prepared using the method essentially as described in Chapman-Smith et al. (1994) Biochem J. 302, 881-887. Proteins were transferred to nitrocellulose, as described by Sambrook, J., Fritsch, E. F., and Maniatis, T., in Molecular Cloning: A Laboratory Manual. Cold Spring Harbor Laboratory Press, NY, Vol. 1, 2, 3 (1989), and biotin containing peptides detected, essentially as described by Salto et al. (1999) Mol Cell Biochem 200:111-7. Expression of each construct was determined by anti-GST Western blot following manufacturer's instructions (Amersham Bioscience).

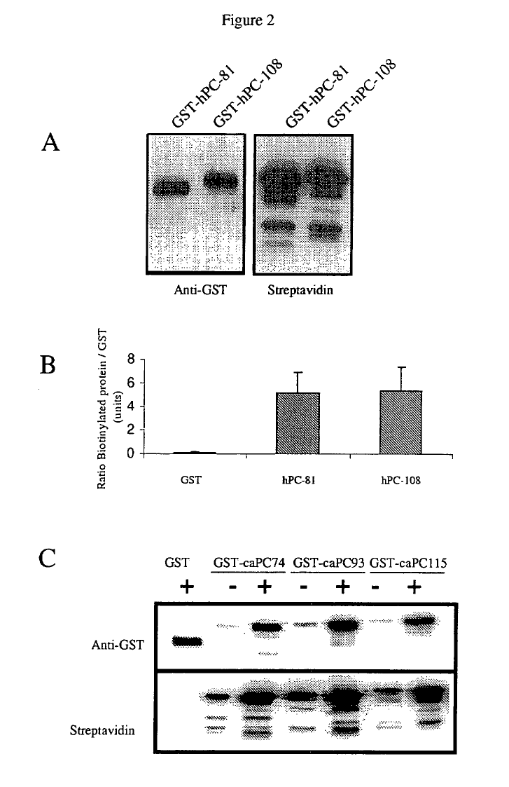

The results of the streptavidin and anti-GST blots for the human pyruvate carboxylase fragments are shown in FIG. 2a. The results of the streptavidin and anti-GST blots for the Candida pyruvate carboxylase fragments are shown in FIG. 2c. Products of the predicted size were detected by both Western using anti-GST antibodies or by streptavidin blot.

Quantitation of the Western Blots for the human pyruvate carboxylase fragments was performed using NIH Inage software version 1.62. The relative biotinylation ratio was calculated by dividing the signal from the streptavidin blot by the signal from the anti-GST Western blot. The results are shown in FIG. 2B.

As can be seen from the data shown in FIG. 2, GST alone showed no evidence of biotin incorporation. However the two human peptides, hPC-81 and hPC-108, were biotinylated (FIG. 2A) implying enough information was contained in the 81 amino acid fragment for correct protein folding. Quantitation of biotinylation, calculated relative to the level of fusion protein expression, revealed that both peptides were equal BPL substrates in vivo. (FIG. 2B)

Similarly, all three peptides from C. albicans pyruvate carboxylase were equivalent biotin protein ligase substrates in the in vivo assay (FIG. 2C). As can be seen from the data, the peptides from both Candida and human were efficiently biotinylated in vivo.

### Example 3

### Purification of Biotin Domains

Each peptide from Candida and human was expressed as a GST fusion protein permitting both high level expression in E. coli and rapid purification by affinity chromatography. To express the fusion proteins for purification, bacterial cultures of BL21 harbouring the pGEX-4T-2 based expression vectors were grown in shake flasks in 2YT supplemented with 100 ug/ml ampicillin. Overnight cultures were diluted 1:100 into 1 litre fresh media and grown at 37° C. to A₆₀₀ 0.6-0.8 before addition of IPTG to a final concentration of 0.1 mM. After 1 h, the cells were harvested by centrifugation, washed in phosphate buffered saline (PBS) and resuspended in 30 ml PBS. Cells were disrupted by two passages through a French Press (42 000 to 60 000 kPa) and the cellular debris removed by centrifugation at 10,000×g for 10 minutes followed by filtration through a 0.45 μm filter.

The prepared lysate was passed over a 1 ml GST-Trap column (Amersham-Biosciences) continuously overnight at 4° C. Unbound material was removed by washing with 10 column volumes of PBS containing 1 mM DTT. The column was equilibrated in 5 volumes of thrombin digestion buffer (20 mM Tris-HCL pH 8.4, 150 mM NaCl, 2.5 mM CaCl₂) before addition of 7.5 U of biotinylated thrombin (Novagen). The GST fusions were cleaved overnight at RT before the cleaved biotin domains were washed off the column in 5 volumes of thrombin digest buffer. Biotinylated thrombin and biotin domain were simultaneously removed from the solution using Streptavidin-Sepharose High Performance (Amersham Biosciences) in a pull-down reaction, following manufacturers instructions. The non-biotinylated material (biotin domain) in the supernatant was collected, dialysed against 2 mM ammonium acetate pH 7.4 and lyophilised.

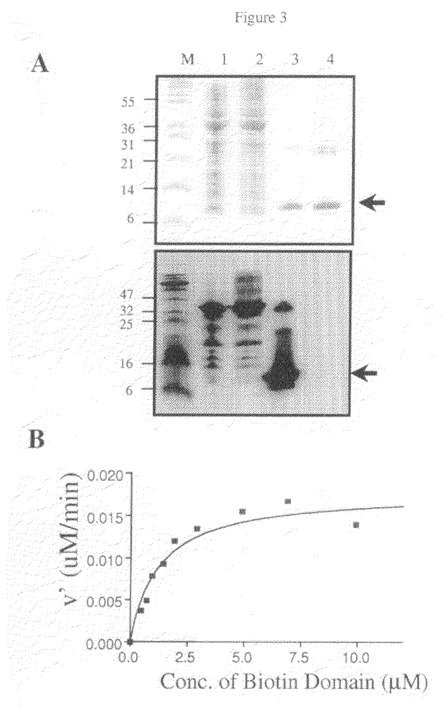

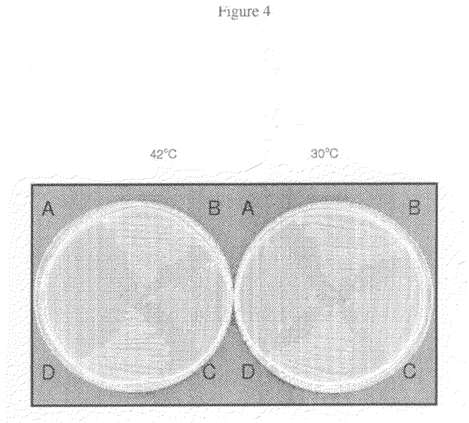

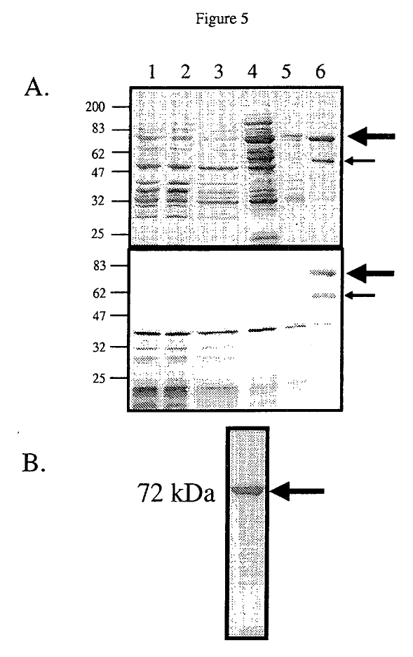

Purification of the human pyruvate carboxylase biotin domain (hpC-108) is shown in FIG. 3A. Material in the unbound fraction is shown in lane 1 and protein in the wash fraction is shown in lane 2. The biotin domain was released from GST by addition of thrombin directly onto the column. Cleaved material (lane 3) was washed off the column and biotin-containing material removed using Streptavidin Sepharose (lane 4). The purification was monitored by SDS-PAGE (top panel) and Streptavidin blot (lower panel). Migration of molecular mass standards (kDa) is shown on the left and the position of hPC-108 shown on the right.

### Example 4

### Purified Biotin Domains are Efficient Substrates for Biotinylation

Fractions of purified hPC-108 prepared as described in Example 2, were fractionated on duplicate 12% polyacrylamide gels under reducing conditions using Tris-tricine running buffer essentially as described in Schagger & von Jagow (1987) Anal. Biochem. 166: 368-79. One gel was used for SDS-PAGE analysis, and the other was used for a Western transfer onto nitrocellulose membrane and probed with Streptavidin-horse radish peroxidase. Evidence of biotinylated fusion protein (38 kDa) and biotinylated biotin domain (12 kDa) were observed on the Streptavidin blot indicating these proteins can function as substrates for E. coli BPL in vivo.

Biotinylated biotin domain was removed from the preparation of the thrombin cleaved hPC-108 using streptavidin sepharose and protein concentration determined using a BCA assay kit following manufacturer's instructions (Pierce).

Purified hPC-108 was analysed in an in vitro biotinylation assay using recombinant human BPL as follows: BPL activity was assayed by measuring the incorporation of [³H]biotin into biotin domain. The reactions contained 50 mM Tris-HCl, pH 8.0, 3 mM ATP, 5.5 mM MgCl₂, 50 mM KCl, 5 uM biotin, 5 pmol [³H]biotin (specific activity 35-44 Ci/mmol) 0.1 mM dithiothreitol, 0.1 mg/ml BSA and varying concentrations of hPC-108. The reaction was initiated by addition of purified yeast BPL to a final concentration of 13 nM and incubated at 37° C. for up to 30 minutes when aliquots of the reaction were spotted onto biotin- and trichloroacetic acid-treated filters. After air-drying, the filters were washed twice in 10% ice cold trichloroacetic acid and once in ethanol, dried and the acid insoluble radioactivity measured. Values for Kₘ and Vₘₐₓ were determined by fitting a plot of substrate concentration against rate to the Michaelis-Menten equation using GraphPad Prism for MacIntosh (GraphPad Software Inc, San Diego, Calif.).

The enzyme velocity was measured with varying concentrations of hPC-108. The K_{M} was determined to be 1.0±0.2 uM indicating that the domain is a good BPL substrate, as shown in FIG. 3B.

### Example 5

### Cloning the C. albicans BPL Gene

The sequence data for Candida albicans BPL was obtained from the Stanford Genome Technology Centre (accession code CAN005069; ORF YDL141w) and is designated SEQ ID. No. 31:

The gene was cloned using polymerase chain reaction (PCR) with genomic DNA from C. albicans (strain CBF 562) as discussed below.

Candida has a rare reassignment of the genetic code where the universal codon for leucine CUG is encoded as a serine. Therefore, it was necessary to alter four CUG codons in the bpl sequence to universal serine codons before recombinant expression of the enzyme in a host such as E. coli.

The DNA manipulations were as follows:

Three separate fragments that spanned the entire gene were initially generated and subcloned into the pGEM-T Easy cloning vector (Promega) thus allowing further modification to the DNA sequences.

A fragment spanning the 5′ half of the gene, encompassing nucleotides 1 to 1016, was obtained using PCR overlap extension. The PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers were included along with 1 ug of genomic DNA as the template. Thermocycling conditions were 30 cycles of 92° C. for 30 seconds, 60° C. for 30 seconds and 72° C. for 45 seconds.

Two smaller genomic PCR products were initially obtained either with oligonucleotides C1/32 and C2/39, or with C3/44 and C4/44:

C1/32

(SEQ ID NO.32)

[5′ATCTACTCATGAATGTTTTAGTATATTCTGGC]
C2/39

(SEQ ID NO.33)

[5′GACGGCTCTGGCGCCAGTACGACTTTCGTACTTGAAACC]
C3/44

(SEQ ID NO.34)

[5′CGTACTGGCGCCAGAGCCGTCAAATTGAGCGTCAATACAGCTGC]
C4/44

(SEQ ID NO.35)

[5′TCCGGTATACCGGCGCTAGTGAGAAAGTTGATATACTTGACTGG]

The products were purified and included as a template for PCR with oligonucleotides C1/32 and C4/44. This produced a 1027 base pair fragment which was cloned into the pGEM-T Easy producing pGEM(C1/C4)Ser. By this approach, a BspH1 restriction site was introduced at the initiation codon and a unique Nar1 restriction site engineered at nucleotide 411. In addition the CUG codons at nucleotides 402 and 432 were altered to AGU and AGC respectively.

A second fragment was obtained that spanned nucleotides 567 to 1016 using genomic PCR with oligonucleotides C5/20 [5′GTGGATrTAGAGAAGGCTGC] (SEQ ID NO. 36) and C4/44. The 449 base pair fragment was subcloned into pGEM-T Easy generating pGEM(C5/C4). An Acc1 restriction site at nucleotide 859 and the CUG codon at position 865 were simultaneously modified using oligonucleotides C10/45 and C11/45 with the Quickchange mutatgenesis protocol (Stratagene) producing pGEM(C5/C4)Ser. A polymorphorism from the published sequence in the species used here was detected by DNA sequencing. Nucleotide 719 was found to be a T base thus introducing another Acc1 site into the gene and changing the codon to an alanine (published sequence contains a valine at the corresponding position). The nucleotide at this position was mutated to C with oligonucleotides C15/40:

(SEQ ID NO.37)

[5′GTGGTTGACACACTTCGAGCATACGATCACAACAAAAAGG]
and;
C16/40:

(SEQ ID NO.38)

[5′CCTTTTTGTTGTGATCGTATGCTCGAAGTGTGTCAACCAC]

producing pGEM(C5/C4)Ser-Acc.

The third of the overlapping fragments encompassed the entire 3′ half of the bpl gene from nucleotides 943 to the termination codon at 1993. This was generated by genomic PCR with oligonucleotide C6/22: [5′ACTAGTGAGTATGTTGGTAGTG] (SEQ ID NO. 39) and; oligonucleotide C7/65: [5′GAGCTCGGTACCTAATGATGATGATGATGATGATGATGATGACCGGTCTT CTTATATACTAAACC] (SEQ ID NO. 40) producing pGEM(C6/C7). The C7/65 primer fused the coding sequence for a glycine-threonine-(histidine)₉ extension onto the C-terminus of the expressed gene product (the glycine-threonine motif introduced a unique Age1 restriction site into the gene). Kpn1 and Sac1 restriction sites were engineered onto the end of the gene to facilitate cloning into appropriate expression vectors.

The 5′ and 3′ ends of the bpl gene were fused together by digesting pGEM(C1/C4)Ser with Spe1 and Sac1 and ligation with similarly treated fragment from pGEM(C6/C7). The sequence between nucleotides 592 to 1007 in the resulting vector was replaced with the modified sequence from pGEM(C5/C4)Ser-Acc using the Acc1 sites at these positions. The final construct, pGEM(caBPL-His₉) contained the full length gene with all the desired modifications. For the recombinant expression of BPL for in vivo complementation assays, the BspH1/Kpn1 fragment from pGEM(caBPL-His₉) was cloned into Nco1/Kpn1 treated vector pARA13 (as described in Cagnon et al. (1991) Prot. Fng. 4:843-847). For high-level expression of BPL for purification from E. coli, the BspH1/Sac1 fragment from pGEM(caBPL-His₉) was cloned into Nco1/Sac1 treated pET-16b (Novogen).

The nucleotide sequence of the C. albicans bpl gene after DNA manipulation is designated SEQ ID No. 41

### Example 6

### Cloning the Human bpl Gene

Sequence data required for cloning of the human bpl gene was obtained from GenBank accession number X80160. The nucleotide sequence of the human bpl gene is designated SEQ ID No. 42.

The cDNA for human BPL was obtained using reverse-transcription and PCR techniques upon human liver total RNA. 5 ug of RNA was heated at 65° C. for 5 minutes with 2 pmol of oligo(dT)₁₈ primer and 3 uM of dNTP mix. Samples were chilled on ice before the addition of DTT to 10 uM, and first strand cDNA synthesis buffer to 1× (Gibco BRL). 40 units of RNAsin and 200 units of Superscript II (Gibco BRL) were added and cDNA synthesis performed at 42° C. for 50 minutes, followed by inactivation of the polymerase at 72° C. for 15 minutes. The cDNA was subsequently employed as a template for PCR. The PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers were included with the cDNA. Thermocycling conditions were 30 cycles of 92° C. for 30 seconds, 60° C. for 30 seconds and 72° C. for 45 seconds.

The gene was obtained as two overlapping fragments, each of which was subcloned into pGEM-T Easy. The 5′ gene fragment, encompassed nucleotides 1-1444, was produced using oligonucleotides FW1:

(SEQ ID NO.43)

[5′ATATAACCATGGAAGATAGACTCCACATGGATAAT]
and;
oligonucleotide RW1:

(SEQ ID NO.44)

[5′GGAGACGCATCGTTGTGG].

PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers were included along with 1 ug of genomic DNA as the template. Thermocycling conditions were 30 cycles of 92° C. for 30 seconds, 60° C. for 30 seconds and 72° C. for 45 seconds.

The 3′ fragment, encompassing nucleotides 1304-2179, was produced using oligonucleotides FW2 [5′CTTGTATACCTGTGGTGACC] (SEQ ID NO. 45) and RW2 [5′ATAATCCCTACTCGAGCTAATGATGATGATGATGATGCCGCCGTTTGGGG AGGATGAGGTTTCT] (SEQ ID NO. 46). PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers were included along with 1 ug of genomic DNA as the template. Thermocycling conditions were 30 cycles of 92° C. for 30 seconds, 60° C. for 30 seconds and 72° C. for 45 seconds.

Primer RW2 introduced nine histidine codons onto the 3′ end of the bpl gene as well as a Xho1 restriction site after the coding sequence. To fuse the 5′ and 3′ ends of the gene, the vector containing the 5′ fragment was digested with Bgl11 and Xho1 and ligated to the similarly treated 3′ fragment producing pGEM(hBPL-His₆). Vector pARA(hBPL-His₆) was produced by cloning the full-length gene into Nco1/Hind111 treated pARA13.

The human bpl gene was further modified to permit recombinant expression of the enzyme with the first 79 amino acids deleted as it has been reported that this variant displays greater activity than full-length BPL.

PCR was performed with oligonucleotide B14/29 [5′AAGGAGAGCCATGGCCTCTGGGAGTGAGC] (SEQ ID NO. 47) and oligonucleotide B15/23 [5′TCCTGTCCTTGTCCTCATTCTCC] (SEQ ID NO. 48) using pGEM(hBPL-His₆) as template. PCR was performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers were included along with 1 ug of genomic DNA as the template. Thermocycling conditions were 30 cycles of 92° C. for 30 seconds, 60° C. for 30 seconds and 72° C. for 45 seconds.

This product encompassed nucleotides 238 to 943 and introduced an Nco1 restriction site at the initiator methionine codon. The PCR product was digested with Nco1 and Kpn1 and ligated into similarly treated pARA(hBPL-His₆). The product, pARA[Met¹-Ala⁸⁰]hBPL-His₆, was used to produce a vector for high level bacterial expression. The Nco1/Hind111 fragment was introduced into similarly treated pET-16b yielding pET[Met¹-Ala⁸⁰]hBPL-His₆.

The nucleotide sequence of the human bpl cDNA after manipulation is designated SEQ ID. No 49.

### Example 7

### Assay of C. albicans and Human BPL Activity In Vivo

In order to determine the biological activity of the Candida and human bpl clones constructed, a series of expression vectors were transformed into E. coli BM4062 (Barker & Campbell (1981) J. Mol. Biol. 146:469-492). This strain contains the birA85 mutation in the bacterial bpl gene which confers a temperature-sensitive phenotype. At the restrictive temperature of 42° C. only those transformed cells expressing a functional exogenous BPL survive.

Plasmids pARA(yBPL-His₆), containing the entire gene for Saccharomyces cerevisiae BPL and expressing a functional yeast biotin protein ligase, pARA(caBPL-His₉), pARA[Met¹-Ala⁸⁰]hBPL-His₆ were transformed into BM4062 and grown at both 30° C. and 42° C. These vectors permit constitutive expression of BPLs from Saccharomyces cerevisiae, C. albicans and H. sapiens BPLs respectively.

As shown in FIG. 4, as expected all strains grew at the permissive temperature. Cells harbouring the parent vector pARA13 failed to grow at the restrictive temperature. All three vectors containing a bpl coding sequence complemented the bacterial mutation at 42° C., indicating that these genes encode functional BPL.

### Example 8

### Purification of Recombinant C. albicans and Human BPLs

Recombinant C. albicans and human BPL were expressed in E. coli and purified by nickel-chelating affinity chromatography.

Bacterial cultures of E. coli BL21(DE3)pLys harbouring the pET-16b based expression vectors were grown in shake flasks in 2YT supplemented with 100 ug/ml ampicillin and 30 ug/ml chloramphenicol. Overnight cultures were diluted 1:100 into 2 l fresh media and grown at 30° C. to A₆₀₀ 0.6-0.8 before addition of IPTG to a final concentration of 0.1 mM. After 3 h, the cells were harvested by centrifugation, washed in binding buffer (20 mM Tris-HCl, pH 7.9, 0.5 M NaCl, 50 mM imidazole) and resuspended in 60 ml binding buffer containing 1 mM PMSF and 100 ug/ml lysozyme. The cell suspension was incubated on ice for 15 minutes before one cycle of freezing at −80° C. and thawing. The cell lysate was sonicated and centrifuged at 10,000×g for 10 minutes. After a second centrifugation, the supernatant was filtered through a 0.45 μm filter prior to chromatography.

His-tagged material was purified on a 5 ml HiTrap Chelating HP column (Amersham-Biosciences). Once the nickel charged column was equilibrated in binding buffer the cell lysate was loaded onto the column at a flow rate of 1 ml/min over 3 hours. The column was washed with 10 column volumes of binding buffer and 10 volumes of wash buffer (20 mM Tris-HCl, pH 7.9, 0.5 M NaCl, 100 mM imidazole) before the bound material was eluted with 3 volumes of elution buffer (20 mM Tris-HCl, pH 7.9, 0.5 M NaCl, 0.25 M imidazole). Fractions containing BPL were pooled and dialysed overnight against 4 l storage buffer (50 mM Tris-HCl, pH 8.0, 1 mM EDTA, 1 mM dithiothreitol, 5% (v/v) glycerol). Fractions containing BPL, detected by SDS-PAGE and Ni-NTA Western blot, were pooled and stored at −80° C. N-terminal sequencing of proteins by automated Edman degradation, to confirm protein purification, was performed using a Perkin-Elmer Procise 492 protein sequencer.

Induction of the protein was performed at 30° C. with low concentrations of IPTG to minimise the production of insoluble inclusion bodies. The BPL, tagged with a multi-histidine sequence at the C-terminus, were rapidly purified in a single step to >90% purity. Binding of the protein extract to the column was performed slowly and in the presence of 50 mM imidazole to improve the yield of enzyme whilst reducing non-specific proteins interacting with the resin. After a wash step containing 100 mM imidazole, the enzyme was eluted with 250 mM imidazole.

Analysis of C. albicans BPL by SDS-PAGE (12% polyacrylamide gel under reducing conditions) and NiNTA blot revealed two bands both containing the C-terminal histidine tag, as shown in FIG. 4A. The most prominant band corresponded to the 78.4 kDa full-length protein, a result confirmed by N-terminal sequencing. A minor species was determined to be a proteolytic product of the enzyme where digestion had occurred between residues phenylalanine 248 and methionine 249. Analysis of the purified human BPL by SDS-PAGE and N-terminal sequencing showed a single major species was produced (FIG. 4B).

### Example 9

### Assay of BPL Activity In Vitro

One hundred ng of biotin domain was coated onto each well of Griener Lumitrac 600 White 96-well plates (Stennick Scientific) with >95% of the peptide binding. Coating was performed overnight at 4° C. in 100 uL of Tris Buffered saline (TBS, pH 7.5). The sample was removed and wells blocked in 150 ul of a 1% BSA solution in TBS for 1 hr at 37° C. The blocking buffer was removed and wells washed five times in TBS buffer containing 0.1% Tween-20. Into each well 85 ul of the BPL reaction mix was added. The reaction for both C. albicans and human BPL are performed in 50 mM Tris-HCl pH 8.0, 100 uM ATP, 5.5 mM MgCl₂, 0.1 mg/ml BSA, 10 uM biotin, 0.1 uM dithiothreitol.

Ten ul of either buffer alone or buffer containing inhibitor was added to the reaction mix and pre-equilibrated at 37° C. for 5 minutes. BPL reactions are initiated by the addition of 5 ul enzyme to a final concentration of 3.4 nM (1-5 nM). The reaction is allowed to proceed at 37° C. for 15 minutes at which point it is terminated by the addition of 5 ul 0.5 M EDTA (final concentration 25 mM). The reaction mix was discarded and wells washed five times in TBS-TD (TBS, 0.1% Tween-20 and 100 uM diethylenetriaminepentaacetic acid (DPTA)). The quantitative analysis of biotinylated protein formed in the reaction and coupled onto the well surface was determined using time resolved fluorescence. Fifty ul of Europium labelled streptavidin (Perkin-Elmer) solution, diluted to 0.1 ug/ml in TBS-TD, was added to each well. The plates were incubated at 37° C. for 1 hr before being washed five times in TBS-TD then three times in water. Fifty ul of DELFIA Enhancement solution (Perkin Elmer) was added and incubated for ten minutes at room temperature before quantitation. Quantitation was performed using a FLUOstar Galaxy plate reader (BMG Labtechnologies).

### Example 10

### Kinetic Analysis of C. albicans BPL

The activity of BPL was investigated using steady state kinetics by assaying the velocity of the enzyme within the first 10% of the reaction. BPL activity was determined by measuring the incorporation of biotin into a biotin-accepting domain adsorbed onto the surface of a 96 well plate. The presence of magnesium ions, ATP, biotin and the non-biotinylated form of a biotin domain were necessary substrates for activity. The inclusion of EDTA inhibited the reaction. Systematic investigation of each reaction component was then performed to determine the conditions required for optimal activity for C. albicans BPL.

A range of buffers (TBS, PBS and Tris) at pHs in the range of 7-8.5 were examined to determine the best conditions for coupling the biotin domains onto the well surface. PBS and TBS buffers performed better than the non-salt Tris buffer, although no significant difference was observed between the buffers. Optimal pH for coating was 7.5-8.0. A TBS buffer at pH 7.5 was chosen as this allowed the use of a single buffer system throughout the entire procedure.

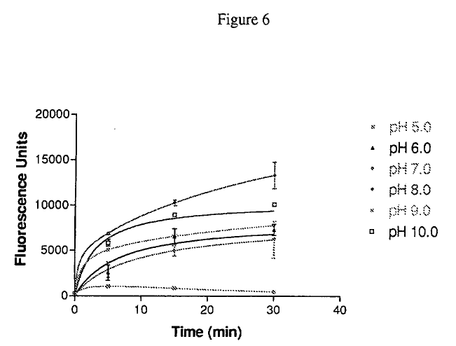

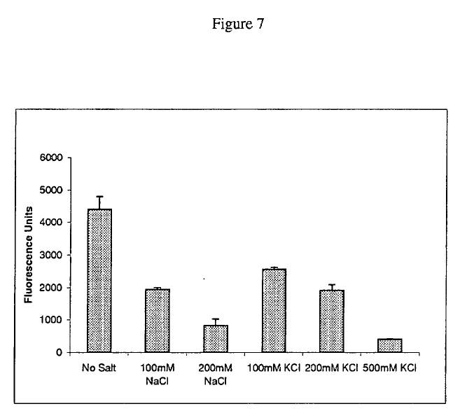

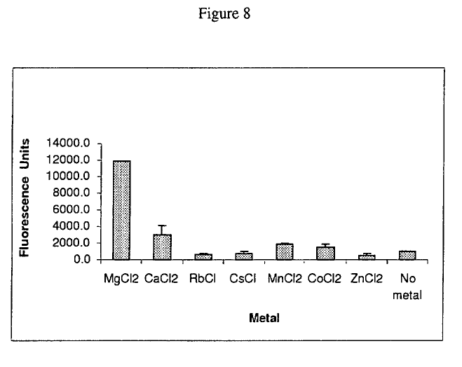

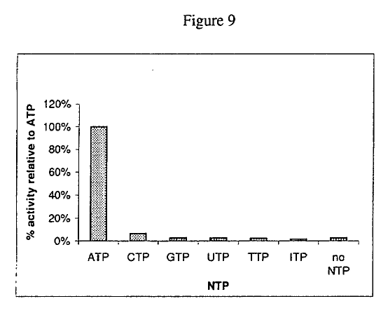

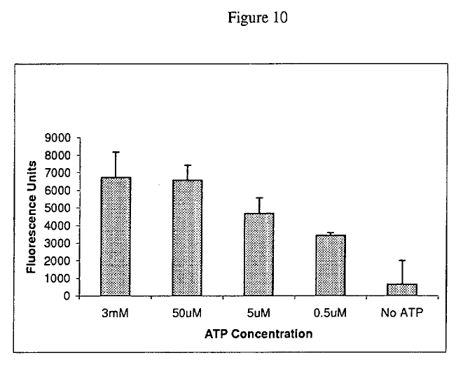

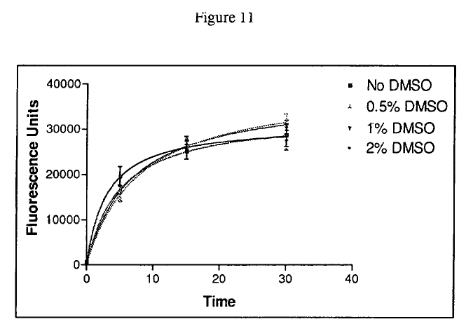

The BPL reaction with C. albicans enzyme was performed in sodium acetate buffer (pH 5.0-6.0), Tris buffer (pH 7.0-8.0) or sodium carbonate buffer (pH 9.0-10.0) in order to determine the optimal pH for the reaction. The C. albicans BPL displayed activity across a broad pH range (pH 6-10) with optimal activity at pH 8.0 (FIG. 6). The addition of salt in the reaction, either NaCl or KCl, was omitted as both were found to be inhibitory (FIG. 7). Magnesium chloride is essential in the reaction and the enzyme only poorly utilised other divalent metal ions tested (FIG. 8). The enzyme requires a source of nucleotide triphosphate but when a series of compounds where assayed only ATP facilitated catalysis (FIG. 9). The enzyme exhibited full activity in the assay with as little as 50 uM ATP (FIG. 10). The BPL was also completely active in 2% dimethyl sulfoxide (FIG. 11).

### Example 11

### Inhibition of BPL Activity

Two potential inhibitors of the BPL reaction were tested in the in vitro biotinylation assay. The first, pyrophosphate, is the product of ATP hydrolysis in the first partial reaction catalysed by BPL. The addition of pyrophosphate should act as a product inhibitor in the reaction thus reducing BPL activity.

The second compound, biotinol-adenylate (BtnOH-AMP), is a derivative of biotinyl-adenylate also formed in the first partial reaction. This non-hydrolysable molecule should inhibit BPL by specifically competing with ATP and biotin for binding in the active site of the enzyme. The structure of this molecule is as follows:

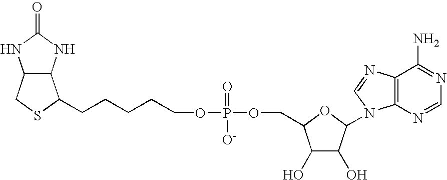

Both pyrophosphate and BtnOH-AMP were potent inhibitors of the Candida BPL reaction.

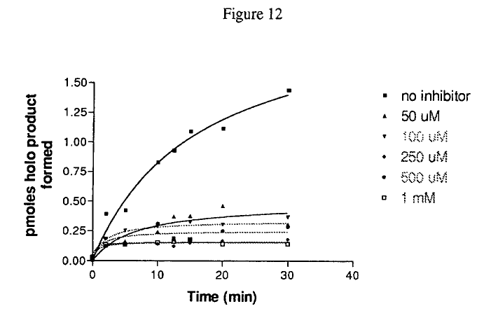

Pyrophosphate strongly inhibited the reaction in the range of 50 uM to 1 mM (FIG. 12).

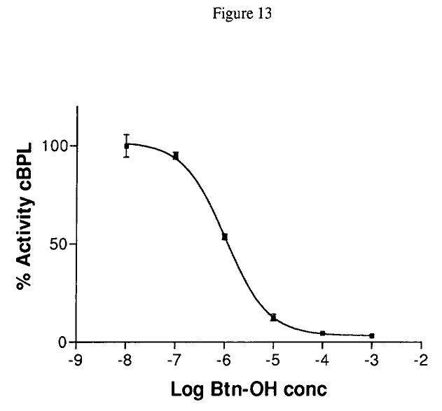

Concentration response curves performed with BtnOH-AMP yielded the low IC₅₀ value of 1.0±0.2 uM (FIG. 13). Together these data show that the assay is suitable for the detection and quantitative analysis of BPL inhibitors.

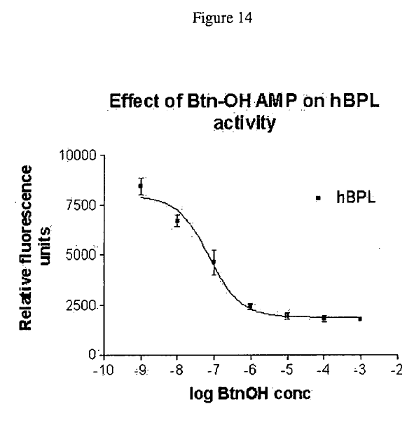

BtnOH-AMP was also found to be an inhibitor of the human BPL reaction (FIG. 14).

### Example 12

### Cloning and Purification of E. coli BPL (BirA)

A methodology for the recombinant expression and purification of the BPL from E. coli (BirA) is as described in Chapman-Smith et al. (2001) Protein Sci 10(12): 2608-17.

The nucleotide sequence for the birA gene is designated SEQ ID. No. 50 (GenBank Accession number M15820).

To introduce the coding sequence for the birA gene into a high level expression vector, the gene will be excised from the plasmid pBA11 (Barker & Campbell (1981) J. Mol. Biol. 146: 469-492) using BspH1 and BstY1. The 1.17 kbp fragment will be ligated into Nco1 and BamH1 treated pET-16b (Novagen) yielding the plasmid pHBA. For protein expression this vector will be introduced into E. coli BL21(DE3) using calcium chloride transformation.

BirA may be purified from E. coli BL21(DE3) cells transformed with the plasmid pHBA, which contains the entire coding region for E. coli BirA, and pET16b (Novagen).

For expression, cells will be revived from storage at −80° C. onto LB agar supplemented with 2% glucose and 200 ug/mL ampicillin and grown overnight at 30° C. Cells may then be harvested from an overnight culture grown at 30° C. in LB containing 2% glucose and 200 ug/mL ampicillin and resuspended in fresh LB containing 200 ug/mL ampicillin or 100 ug/mL carbenicillin and 25 mL aliquots may then be used to inoculate 500 mL volumes of the same media. Cultures will be grown in shaker flasks at 30° C. to A₆₀₀ₙₘ of 0.5, and then transferred to 37° C. and expression induced by addition of isopropyl-1-thio-β-D-galactopyranoside (IPTG) to a final concentration of 0.1 mM.

After 2-3 h cells are harvested, washed and resuspended in 50 mM sodium phosphate, pH 6.0, 50 mM KCl, 5% glycerol, 0.1 mM dithiothreitol (Buffer A), and lysed with a French pressure cell. Cell-free extract may then be applied directly to a 50 mL S-Sepharose Fast-Flow column (Amersham Biosciences) equilibrated in Buffer A and the BirA protein eluted with a linear gradient of 50-500 mM KCl. Fractions containing enzymatic activity elute at around 300 mM KCl and coincide with the major absorbance peak at 280 nm. This material is pooled and dialysed overnight against 20 mM Tris-HCl, pH 8.0, 5% glycerol, 0.1 mM dithiothreitol, at 4° C., and applied to a Q-Sepharose Fast-Flow column (Amersham Biosciences) equilibrated in the same buffer and the protein eluted with a linear gradient of 0-400 mM KCl. Fractions containing enzymatic activity elute at around 120 mM KCl and coincide with die major absorbance peak at 280 nm. This material is then dialysed overnight against 20 mM Tris-HCl, pH 7.5, 200 mM KCl, 5% glycerol, 0.1 mM dithiothreitol, at 4° C. and stored at −80° C.

### Example 13

### Substrate for E. coli BPL

E. coli contains only a single biotinylated protein, the biotin carboxyl carrier protein (BCCP). BCCP is a component of the multi subunit enzyme acetyl CoA carboxylase (ACC).

Sequence data for the BCCP gene may be obtained from GeneBank, accession number M80458.

The biotin domain from this enzyme is contained within the 87 C-terminal residues of BCCP (BCCP-87), as follows:

(SEQ ID NO. 51)

M⁷⁰EAPAAAEISGHIVRSPMVGTFYRTPSPDAKAFIEVGQKVNVGDTLCI
VEAMKMMNQIEADKSGTVKAILVESGQPVEFDEPLVVIE¹⁵⁶

This fragment will be expressed in E. coli by using PCR to generate the appropriate DNA fragment for cloning into pGEX-4T-2. DNA encoding each domain will be amplified by PCR using oligonucleotides that engineer a BamH1 restriction site at the 5′ end of the DNA fragment and an EcoR1 site at the 3′ end (the restriction sites are underlined in the primer sequences). By cloning the BamH1 and EcoR1 treated PCR product into similarly treated pGEX4T-2, vectors for the expression of each peptide as a C-terminal extension to GST will be produced.

The PCR will employ plasmid pLS141 as described in Li and Cronan (1992) J. Mol. Biol. 267:855-863 with primer B1/32 [5′ ATCTACGGATCCATGGAAGCGCCAGCAGCAGC] (SEQ ID NO. 52) and primer B2/34 [5′ATCTACGAATTCATCACTCGATGACGACCAGCGG] (SEQ ID NO. 53). PCR will be performed with 2.5 units PfuTurbo DNA polymerase (Stratagene) in 200 uM of each dNTP, 10 mM KCl, 10 mM (NH₄)₂SO₄, 20 mM Tris-HCl (pH 8.75), 2 mM MgSO₄, 0.1% Triton X100 and 0.1 mg/ml BSA. Between 0.1 and 0.5 uM of the required oligonucleotide primers will be included along with 1 ug of genomic DNA as the template. Thermocycling conditions to be employed are 30 cycles of 92° C. for 30 seconds, 60° C. for 30 seconds and 72° C. for 45 seconds.

The peptide will be produced in Escherichia coli by expressing peptides encompassing the predicted biotin domain as a fusion to GST. This strategy permits rapid quantitation of both expression of the fusion protein by Western blot using anti-GST antibodies and biotinylation of the substrates by Streptavidin blot.

The biotin domain will be expressed as a GST fusion protein permitting both high level expression in E. coli and rapid purification by affinity chromatography. Bacterial cultures of BL21 harbouring the pGEX-4T-2 based expression vectors will be grown in shake flasks in 2YT supplemented with 100 ug/ml ampicillin. Overnight cultures are diluted 1:100 into 1 l fresh media and grown at 37° C. to A₆₀₀ 0.6-0.8 before addition of IPTG to a final concentration of 0.1 mM. After 1 h, the cells are harvested by centrifugation, washed in phosphate buffered saline (PBS) and resuspended in 30 ml PBS. Cells will be disrupted by two passages through a French Press (42 000 to 60 000 kPa) and the cellular debris removed by centrifugation at 10,000×g for 10 minutes followed by filtration through a 0.45 μm filter.

The prepared lysate will be passed over a 1 ml GST-Trap column (Amersham-Biosciences) continuously overnight at 4° C. Unbound material is removed by washing with 10 column volumes of PBS containing 1 mM Dithiothreitol. The column is then equilibrated in 5 volumes of thrombin digestion buffer (20 mM Tris-HCL pH 8.4, 150 mM NaCl, 2.5 mM CaCl₂) before addition of 7.5 U of biotinylated thrombin (Novagen). The GST fusions are cleaved overnight at RT before the cleaved biotin domains are washed off the column in 5 volumes of thrombin digest buffer. Biotinylated thrombin and biotin domain can be simultaneously removed from the solution using Streptavidin-Sepharose High Performance (Amersham Biosciences) in a pull-down reaction, following manufacturers instructions. The non-biotinylated material, or biotin domain, in the supernatant will be collected, dialysed against 2 mM ammonium acetate pH 7.4 and lyophilised. The material will be analysed by SDS-PAGE and streptavidin blot and quantitated using in vitro biotinylation assay and BCA protein assay kit (Pierce).

### Example 14

### Cloning Arabidopsis thaliana HCS-1

A. thaliana contains two genes encoding different BPLs, hcs-1 and hcs-2, known in plants as holocarboxylase synthetase (HCS). The two gene products share a high degree of identity (82%) and possess amino acid motifs conserved amongst all BPLs in the catalytic core of the enzyme. HCS-1 is the putative plastid form of the enzyme and hence is the enzyme responsible for biotinylation of acetyl-coA carboxylase in this cellular compartment. In plants, the de novo synthesis of fatty acids occurs primarily in the plastid, thus implicating HCS-1 as the most essential of the two BPLs.

The DNA sequence encoding the HCS-1 gene is designated in SEQ. ID. No. 54 (GenBank accession number U41369).

The hcs-1 gene will be obtained as cDNA clone by reverse transcription (RT) and PCR, essentially as described in Sambrock, Fritsch, Maniatis (1989) Molecular Cloning, Cold Spring Harbor Laboratory Press.

Isolation of polyA mRNA from A. thaliana will be prepared using the Straight A's mRNA Isolation System (Novagen). First strand cDNA synthesis will be performed using 250 ng of mRNA in a final volume of 20 ul using Oligo(dT)20 primers (Thermoscript RT-PCR System, Life Technologies). The PCR will subsequently use 1 ul aliquots of RT together with oligonucleotides AbplFor: [5′ ATCTATCCATGGAAGCAGTTCGTTCAACAACAACC] (SEQ ID NO. 55) and; AbplRev: [5′ATCCATGGATCCTAATGATGATGATGATGATGATGATGATGACCGGTTAT TTTTCTTCGAACCAGACC] (SEQ ID NO. 56). These primers will introduce an Nco1 site at the 5′ end of the gene and a BamH1 site at the 3′ end respectively. In addition primer AbplRev introduces a threonine-glycine-(histidine)₉ sequence onto the C-terminus of the gene product, facilitating purification of the protein using nickel chelating chromatography techniques. Digestion of the PCR product with Nco1 and BamH1 will permit cloning of hcs-1 into similarly treated pET-16b. The enzyme will be recombinantly expressed in a bacterial host and the multi-histidine motif will permit rapid purification of the enzyme by metal ion chelating chromatography.

The predicted sequence after DNA manipulation is designated SEQ ID No. 57.

### Example 15

### Substrate for A. thaliana BPL

Dicotyledonous plants contain two structurally different acetyl CoA carboxylases (ACC). The type I ACC is a single polypeptide 220 kDa form, whereas type II is a multi subunit complex analogous to that of E. coli.

Sequence data for the BCCP gene of type H ACC may be obtained from GenBank, accession number U23155.

The biotin domain from this enzyme is contained within the 94 C-terminal residues of BCCP (BCCP-94), as follows:

(SEQ ID NO.58)

P⁹²PSPPTPAKSSLPTVKSPMAGTFYRSPAPGEPPFIKVGDKVQKGQVLC
IVEAMKLMNEIESDHTGTVVDIVAEDGKPVSLDTPLFVVQPVESAP²⁸⁰

This fragment will be expressed in E. coli by using PCR to generate the appropriate DNA fragment for cloning into pGEX-4T-2. DNA encoding each domain will be amplified by PCR using oligonucleotides that engineer a BamH1 restriction site at the 5′ end of the DNA fragment and an EcoR1 site at the 3′ end (the restriction sites are underlined in the primer sequences). By cloning the BamH1 and EcoR1 treated PCR product into similarly treated pGEX4T-2, vectors for the expression of each peptide as a C-terminal extension to GST will be produced.

The domain will be obtained using RT-PCR. Plant cDNA will be obtained as described in Example 14. PCR will be performed using cDNA as a template and primers AaccFor [5′ ATCTACGGATCCCCACCATCCCCACCTACTCC] (SEQ ID NO. 59) and AaccRev [5′ ATCTACGAATTCATCATGGTGCCGATTCTACGG] (SEQ ID NO. 60).

The peptide will be produced in Escherichia coli by expressing peptides encompassing the predicted biotin domain as a fusion to GST. This strategy permits rapid quantitation of both expression of the fusion protein by Western blot using anti-GST antibodies and biotinylation of the substrates by Streptavidin blot.

The biotin domain will be expressed as a GST fusion protein permitting both high level expression in E. coli and rapid purification by affinity chromatography. Bacterial cultures of BL21 harbouring the pGEX-4T-2 based expression vectors will be grown in shake flasks in 2YT supplemented with 100 ug/ml ampicillin. Overnight cultures are diluted 1:100 into 1 l fresh media and grown at 37° C. to A₆₀₀ 0.6-0.8 before addition of IPTG to a final concentration of 0.1 mM. After 1 h, the cells are harvested by centrifugation, washed in phosphate buffered saline (PBS) and resuspended in 30 ml PBS. Cells will be disrupted by two passages through a French Press (42 000 to 60 000 kPa) and the cellular debris removed by centrifugation at 10,000×g for 10 minutes followed by filtration through a 0.45 μm filter.

The prepared lysate will be passed over a 1 ml GST-Trap column (Amersham-Biosciences) continuously overnight at 4° C. Unbound material is removed by washing with 10 column volumes of PBS containing 1 mM Dithiothreitol. The column is then equilibrated in 5 volumes of thrombin digestion buffer (20 mM Tris-HCL pH 8.4, 150 mM NaCl, 2.5 mM CaCl₂) before addition of 7.5 U of biotinylated thrombin (Novagen). The GST fusions are cleaved overnight at RT before the cleaved biotin domains are washed off the column in 5 volumes of thrombin digest buffer. Biotinylated thrombin and biotin domain can be simultaneously removed from the solution using Streptavidin-Sepharose High Performance (Amersham Biosciences) in a pull-down reaction, following manufacturers instructions. The non-biotinylated material, or biotin domain, in the supernatant will be collected, dialysed against 2 mM ammonium acetate pH 7.4 and lyophilised. The material will be analysed by SDS-PAGE and streptavidin blot and quantitated using in vitro biotinylation assay and BCA protein assay kit (Pierce).

### Example 16

### Cloning of Drosophila BPL

The calculated gene product for Drosophila melanogaster BPL is the largest member of the enzyme family so far recorded. This gene contains 1042 amino acids yielding a 120 kDa protein. Bacteria is not a favourable expression system for the production of large proteins so another expression system will be used. The baculovirus expression system will be employed using insect cells as the host together with the vector pFastBac HT (Invitrogen).

Sequence data required for the construction of a D. melanogaster BPL expression vector was obtained from GenBank, accession number AE003602, and is designated SEQ. ID. No. 61.

For construction, the full-length cDNA clone BcDNA:RE09732 will be obtained from ResGen (Invitrogen). This will allow amplification of the bpl cDNA by PCR using oligonucleotides DbplFor [5′ GAATTCATGTTGACCCTGTATTACGTGAG] (SEQ ID NO. 62) and DbplRev [5′ AAGCTTCATTTCACTATTGATACTTGG] (SEQ ID NO. 63) and the DNA-Polymerase containing proof reading activity Pwo. These primers introduce EcoR1 and Hind111 restriction sites (sequences underlined) at the 5′ and 3′ ends of the cDNA respectively. The PCR product will be subcloned into pGEM-T Easy for DNA sequencing. The product will be subcloned as a single EcoR1/Hind111 DNA fragment into similarly treated pFastBac HT. Expression of the BPL in this system will yield a full-length product containing a MSYYHHHHHHDYDIPTTENLYFQGAMDPEF (SEQ ID NO. 64) N-terminal extension. The hexa-histidine motif will permit rapid purification of the enzyme by metal ion chelating chromatography. The nucleotide sequence of the DNA after manipulation is designated SEQ. ID. No. 65:

### Example 17

### Substrate for Drosophila BPL

A biotin domain from a Drosophila biotin-enzyme has not yet been reported so the most likely candidate to trial is a fragment containing the 125 C-terminal residues of pyruvate carboxylase.

Sequence data for the D. melanogaster pyruvate carboxylase gene may be obtained from GenBank, accession number NM 136683.

The biotin domain from this enzyme is contained within the 125 residues of pyruvate carboxylase (dPC-125), as follows:

(SEQ ID NO.66)

G¹⁰⁵⁷KTLSVKALAVSADLKPNGIREVFFELNGQLRAVHILDKEAVKEIH
VHPKANKSNKSEVGAPMPGTVIDIRVKVGDKVEKGQPLVVLSAMKMEMVV
QSPLAGVVKKLEIANGTKLEGEDLIMIIE¹¹⁸¹

This fragment will be expressed in E. coli by using PCR to generate the appropriate DNA fragment for cloning into pGEX-4T-2. DNA encoding each domain will be amplified by PCR using oligonucleotides that engineer a BamH1 restriction site at the 5′ end of the DNA fragment and an EcoR1 site at the 3′ end (the restriction sites are underlined in the primer sequences). By cloning the BamH1 and EcoR1 treated PCR product into similarly treated pGEX-4T-2, vectors for the expression of each peptide as a C-terminal extension to GST will be produced.

The domain will be obtained from the full-length cDNA clone BcDNA:GH06348 ResGen (Invitrogen). This will be used as the template in a PCR with primers DpcFor [5′ ATCTACGGATCCGGTAAGACGCTGAGCGTGAAAGC] (SEQ ID NO. 67) and DpcRev [5′ ATCTACGAATTCCTATTCGATAATCATAATGAGGTCC] (SEQ ID NO. 68).

The peptide will be produced in Escherichia coli by expressing peptides encompassing the predicted biotin domain as a fusion to GST. This strategy permits rapid quantitation of both expression of the fusion protein by Western blot using anti-GST antibodies and biotinylation of the substrates by Streptavidin blot.

The biotin domain will be expressed as a GST fusion protein permitting both high level expression in E. coli and rapid purification by affinity chromatography. Bacterial cultures of BL21 harbouring the pGEX-4T-2 based expression vectors will be grown in shake flasks in 2YT supplemented with 100 ug/ml ampicillin. Overnight cultures are diluted 1:100 into 1 l fresh media and grown at 37° C. to A₆₀₀ 0.6-0.8 before addition of IPTG to a final concentration of 0.1 mM. After 1 h, the cells are harvested by centrifugation, washed in phosphate buffered saline (PBS) and resuspended in 30 ml PBS. Cells will be disrupted by two passages through a French Press (42 000 to 60 000 kPa) and the cellular debris removed by centrifugation at 10,000×g for 10 minutes followed by filtration through a 0.45 μm filter.

The prepared lysate will be passed over a 1 ml GST-Trap column (Amersham-Biosciences) continuously overnight at 4° C. Unbound material is removed by washing with 10 column volumes of PBS containing 1 mM Dithiothreitol. The column is then equilibrated in 5 volumes of thrombin digestion buffer (20 mM Tris-HCL pH 8.4, 150 mM NaCl, 2.5 mM CaCl₂) before addition of 7.5 U of biotinylated thrombin (Novagen). The GST fusions are cleaved overnight at RT before the cleaved biotin domains are washed off the column in 5 volumes of thrombin digest buffer. Biotinylated thrombin and biotin domain can be simultaneously removed from the solution using Streptavidin-Sepharose High Performance (Amersham Biosciences) in a pull-down reaction, following manufacturers instructions. The non-biotinylated material, or biotin domain, in the supernatant will be collected, dialysed against 2 mM ammonium acetate pH 7.4 and lyophilised. The material will be analysed by SDS-PAGE and streptavidin blot and quantitated using in vitro biotinylation assay and BCA protein assay kit (Pierce).

### Example 18

### Hydroxylamine as a Substrate for Biotin Protein Ligase

Biotin protein ligases also contain biotinyl-AMP synthetase activity (i.e. the first partial reaction) which can be quantitated independently of the second partial reaction.

The product of the first partial reaction, biotinyl-5′-AMP may react with a non-protein amine containing molecule such as hydroxylamine. When BPL is incubated with radiolabelled biotin, MgATP and hydroxylamine, labelled biotinyl-hydroxamate is formed. Unreactive biotin may be removed from the reaction by anion exchange resin. Product formation is measured by quantitating the amount of radioactivity in the cleared supernatant fraction.

To assay for biotinyl-AMP synthetase activity, a reaction mixture containing 50 uM Tris-HCl pH8.0, 12 mM MgCl₂, 12 mM ATP, 0.4 M hydroxylamine, and 33-52.6 Ci/mmol d-[8,9-³H]biotin is prepared. The biotinyl-AMP synthetase activity is initiated by the addition of BPL and terminated by the addition of 100 mM EDTA or heating the sample to 80° C. for 10 minutes. An excess quantity of anion exchange resin, such as AG-X2 (Bio-Rad) or Q-Sepharose (Amersham Biosciences) is added to the reaction to remove [³H]biotin. After shaking the suspension and pelleting the resin by centrifugation, a sample of the supernatant is removed for quantitation of [³H]biotinhydroxamate by liquid scintillation counting.

Accordingly, in determining whether a test compound is an inhibitor of a biotin protein ligase, a molecule such as hydroxylamine may be employed as a substrate for a biotin protein ligase, using the above assay.

### Example 19

### Determination of the Extent of Inhibition of biotinylation of a Protein Substrate In Vivo by Biotinol-Adenylate

Human NIH3T3 fibroblasts will be grown at 37° C. and 100% humidity, 5% CO₂ in 96 well plates with Dulbecco's modified Eagle's medium containing 7.5% foetal calf serum, L-glutamine and penicillin/streptomycin.

Confluent monolayers of fibroblasts will be subjected to treatment with the BPL inhibitor biotinol-adenylate by addition of the compound into the growth media for varying times from 12-72 hours, at concentrations ranging from 10 nM-100 uM. After an appropriate period of time the media will be removed and cells washed with saline to remove excess inhibitor.

To determine if the inhibitor prevents in vivo biotinylation, cells will be lysed with Trizol reagent (Life Technologies) and the protein fraction extracted. This material will be fractionated by SDS-PAGE and used for Western transfer. Duplicate blots will be probed with both anti pyruvate carboxylase antibodies and streptavidin. The level of biotin incorporation into pyruvate carboxylase in vivo will be compared between treated and untreated controls.

The toxicity of the compound will be assessed using the neutral red assay (Sigma). This assay measures lysosomal membrane stability in terms of the retention time of the neutral red dye only within the lysomes of viable cells. Absorbance of converted dye is spectroscopically determined by absorbance at 540 nm.

### Example 20

### Inhibition of BPL from Escherichia coil

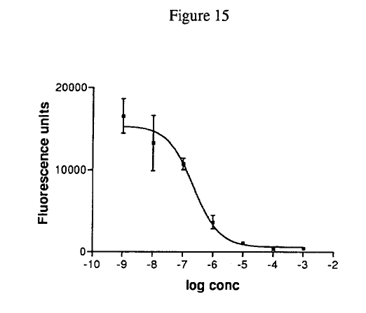

FIG. 15 shows the concentration response curve of bacterial BPL activity inhibition by biotinol-adenylate. The assay for bacterial BPL was performed as described in Example 9. The activity of the enzyme was determined in the presence of varying concentrations of biotinol-adenylate.

### Example 21

### Assay for Inhibitors of BPL Activity In Vitro

The assay for Candida BPL was performed as described in Example 9. A proprietary library consisting of 329 different compounds was obtained. Each compound in the screening library was dissolved in 100% DMSO, added to the reactions to a final concentration of 10 μM or 50 μM and their effect on BPL activity measured. The graph shows the average of the reactions performed in triplicate. The inhibitory activity of the compounds was determined relative to the negative control reactions containing only DMSO. The graph shows the activity of each compound assayed at 10 uM (Grey bars) and 50 uM (black bars).

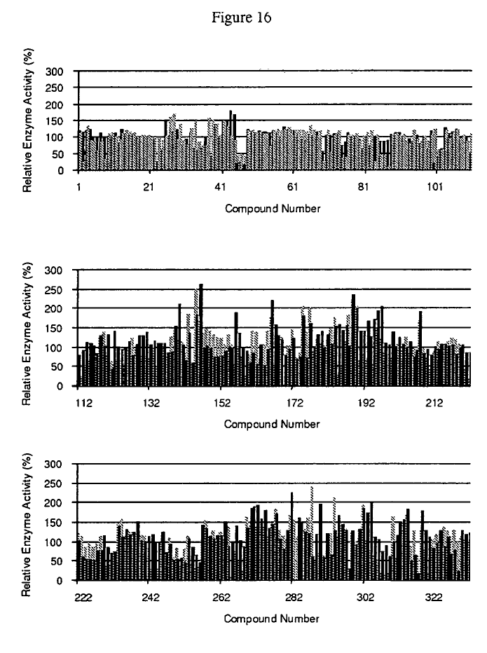

As can be seen from the results shown in FIG. 16, no significant inhibitory activity was observed for most compounds tested at both concentrations.

A small number of compounds showed significant inhibitory activity with Candida BPL at both 10 μM and 50 μM concentrations, exemplified by compounds 69 and 296.

### Example 22

### Differential Inhibition of Candida and Human BPL

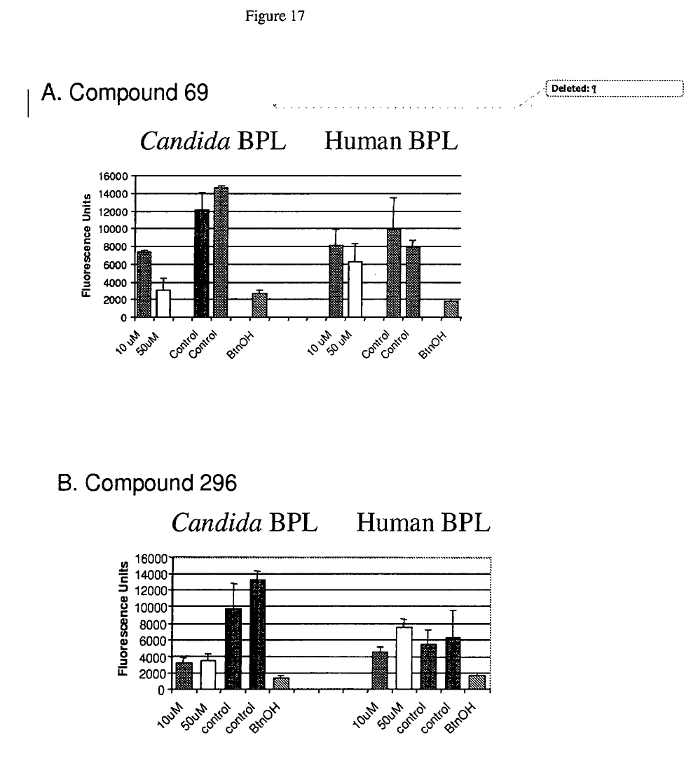

The assay for Candida and human BPLs was performed as described in Example 9. Compounds 69 and 296 were dissolved in 100% DMSO, added to the reactions to a final concentration of 10 μM or 50 μM and their effect on BPL activity measured. The data for compound 69 is shown in Panel A of FIG. 17 and the data for compound 296 is shown in Panel B.

The graphs shows the average and standard error from reactions performed in triplicate. The inhibitory activity of the compounds was determined relative to the negative control reactions containing only DMSO (blue bars). Biotinol-AMP was included in the reactions as a control for inhibition.

Compound 69 inhibited Candida BPL to 23% of full activity at a concentration of 50 uM. At the same concentration human BPL was not affected by this compound. Likewise compound 296 reduced the pathogen's enzyme activity by 81% at a concentration of 10 uM without affecting the human enzyme. These levels of inhibition are comparable to those observed using the potent BPL inhibitor biotinol-AMP at 100 uM.

### Example 23

### Methodology for In Silico Drug Screening

The X-ray structures of BPL from Escherichia coli, in the absence (Wilson et al (1992) “Escherichia coli biotin holoenzyme synthetase/bio repressor crystal structure delineates the biotin- and DNA-binding domains” Proc. Natl. Acad. Sci. USA. 89; 9257-9261) and presence of biotin (Weaver et al (2001) “Corepressor-induced organization and assembly of the biotin repressor: A model for the allosteric activation of a transcriptional regulator” Proc Natl. Acad. Sci. USA. 98; 6045-6050) were used to identify potential inhibitory compounds using in silico technology. Missing amino acids and hydrogen atoms in the structure (PDB: IBIB) were modelled using the Biopolymer module of SYBYL v7.0 (Tripos, Inc. SYBYL, Version 7.0, St. Louis, Mo. 2004). Modelled sections of the protein (encompassing amino acids at positions 2, 18, 24, 27, 33, 38, 51, 56, 77, 140, 141, 211, 223, 267 and 294) were refined by conjugate gradient energy minimisation in explicit water using the program NAMD (Linge et al (2003) “Refinement of protein structures in explicit solvent” Proteins 50; 496-506; Kale et al. (1999) “NAMD2: greater scalability for parallel molecular dynamics” J. Comput. Phys. 151: 283-312). Partial charges for BPL and the Jul. 24, 2004 “lead like” compound library of ZINC (Zinc is not Commercial) were calculated using Biopolymer and the SYBYL Programming Language (SPL) respectively. Amino acid residues 91, 112, 116, 117, 118, 119, 121, 175, 183, 188, 189 and 205 were defined as the active site of BirA and a negative image constructed with ATPTS 2001 (Moreno et al. (2002) “Geometric and chemical patterns of interaction in protein—ligand complexes and their application in docking” Proteins 47: 1-13). The ZINC compound library was then docked with DOCK v5.1.0. (Ewing et al. (2001) “DOCK 4.0: search strategies for automated molecular docking of flexible molecule databases” J. Comput. Aided Mol. Des. 15: 411-428). Poses were scored using Scorer v1.3 and ranked with a threshold of 10% in an in-house consensus scoring program based on the CScore module of SYBYL. Compounds with a score of 6 and the top compounds based on the internal Dock Energy Score were visually inspected and non-viable candidates filtered out.

Using this in silico methodology, eight compounds were identified that inhibited Escherichia coli BPL, four with low nanomolar affinity (data shown for compounds 2-5 below), and another in the micromolar range. Compounds 24 also demonstrated inhibitory properties when assayed against Staphylococcus aureus BPL. Enzymes from humans and the yeast Saccharomyces cerevisiae were not inhibited by compounds 2-5 when assayed at concentrations less than 50 μM. Together these data demonstrate that it is possible to identify differential inhibitions of biotin protein ligase.

Compound 4 identified in the screen was 2-allyl-2-(3,5-dimethoxybenzyl)malonic acid:

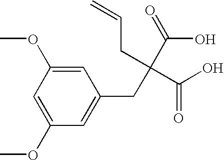

### Example 24

### Differential Inhibition of BPLs

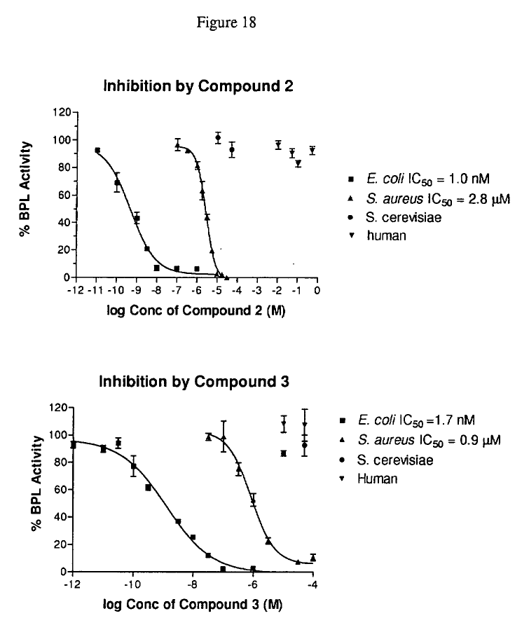

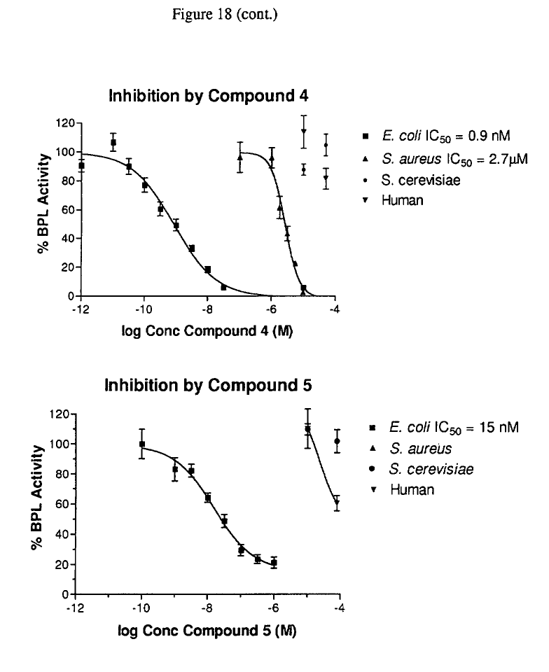

The activity of BPL from Escherichia coli, Staphylococcus aureus, Saccharomyces cerevisiae and Homo sapiens was measured using an in vitro biotinylation assay in the presence of increasing concentrations of Compounds 2 to 5. The data is shown in FIG. 18.

The compounds were found to differentially inhibit the two bacterial enzymes without any significant effect on the mammalian and yeast enzymes. The half maximal inhibitory concentrations (IC₅₀) were as follows:

### Compound 2:

E. coli IC₅₀: 1.0 nM
S. aureus IC₅₀: 2.8 μM
S. cerevisiae IC₅₀: >50 μM

### Human IC₅₀: >50 μM

### Compound 3:

E. coli IC₅₀: 1.7 nM
S. aureus IC₅₀: 0.9 μM
S. cerevisiae IC₅₀: >50 μM]

### Human IC₅₀: >50 μM

### Compound 4:

E. coli IC₅₀: 0.9 nM
S. aureus IC₅₀: 2.7 μM
S. cerevisiae IC₅₀: >50 μM

### Human IC₅₀: >50 μM

### Compound 5:

E. coli IC₅₀: 15 nM
S. aureus IC₅₀: >50 μM]
S. cerevisiae IC₅₀: >50 μM

### Human IC₅₀: >50 μM

### Example 24

### Antibiotic Activity of Compound 2

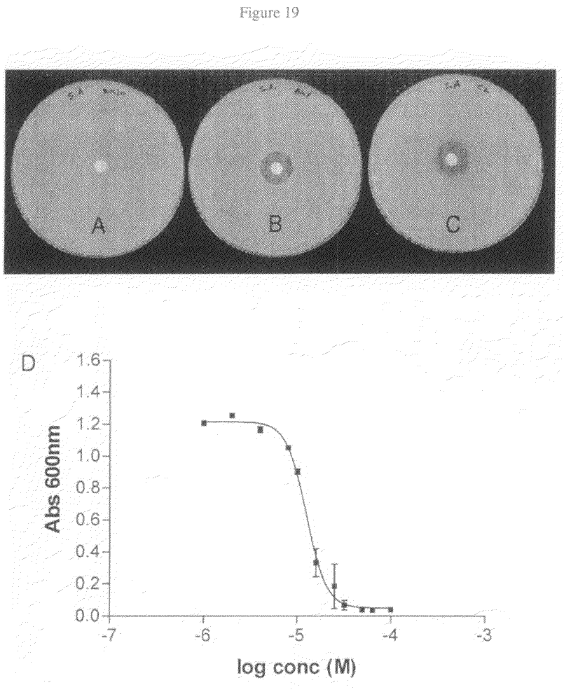

The antibacterial properties of Compound 2 were assessed against Staphylococcus aureus grown on both solid media (A-C) and in liquid culture (D). The data is shown in FIG. 19. Log phase culture was plated onto L-agar and filter discs soaked in A) DMSO (vehicle control), B) 100 μM ampicillin or C) 100 μM compound #2. A single filter was placed in the centre of a bacterial plate before being placed at 37° C. overnight. The clear zone surrounding the disc in B & C represents sensitivity of S. aureus to the treatment. D) This sensitivity was quantitated in liquid culture by seeding 10⁴ cells in a 96 well plate followed by overnight growth at 37° C. The growth of the culture was measured by absorbance at 600 nm. The half maximal effective dose (ED50) was determined to be 11.6±1.3 μM.

### Example 25

### Toxicity Data on Compounds 2 to 5

Hep3B2-1-17 cells were seeded with 1000 cells in 50 μL cell culture medium in 96-well microtiter plates. 50 μL of cell culture medium containing the test compound(s) was added to each well twenty four hours after seeding. Stock solutions of compounds 2-5 in DMSO, were serially diluted in culture medium and added to triplicate wells, producing final concentrations ranging from 40 μM to 0.15625 μM. The final DMSO concentration in the incubation mixture was less than 0.05% (v/v) for the highest concentration test article and respectively lower for die lower concentration test articles. Proliferation of cells was assessed 72 hours post compound addition, using the fluorimetric Alamar Blue (CellTiter Blue) test. This assay indicates cell viability by measuring the ability of cells in culture to reduce resazurin to resorufin, whereby the intensity of the fluorescence signal is directly proportional to the number of live cells and hence an indirect indicator of cell proliferation. Following addition of Cell Titer-Blue Reagent (20 μL/well) and brief mixing, cells were incubated for another 4 hours before fluorescence was measured (Ex/Em of 560/590 nm) using a Spectramax Gemini XPS microplate reader (Molecular Devices, Surrey Hills, VIC, Australia).

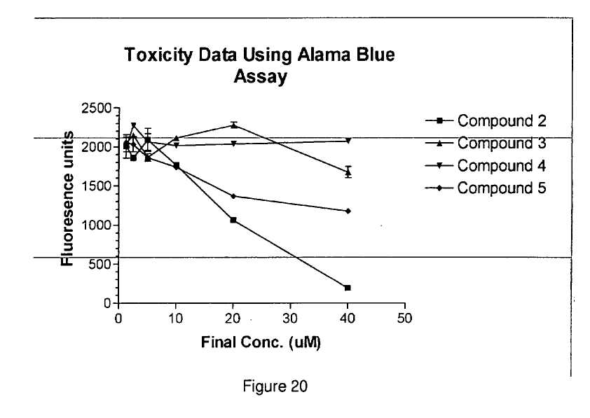

The data is shown in FIG. 20. Compounds 2 to 5 were well tolerated by HEP3B2-1-7 cells, however compound 2 demonstrated moderate toxicity at the highest concentration tested. The other compounds did not appear to demonstrate any toxicity at the specified concentrations.

Finally, it will be appreciated that various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the present invention.

1-57. (canceled)

58. A method of identifying an inhibitor of a biotin protein ligase, the method including:
providing a substrate, wherein the substrate may be biotinylated;
contacting the substrate with biotin and a biotin protein ligase in the presence of a test compound;
determining the extent of biotinylation of the substrate by the biotin protein ligase in the presence of the test compound; and
identifying the test compound as an inhibitor of the biotin protein ligase by a reduction in the biotinylation of the substrate in the presence of the test compound as compared to the extent of biotinylation of the substrate in the absence of the test compound.

59. A method according to claim 58, wherein the biotin protein ligase is a biotin protein ligase of a pathogenic organism of a human, animal or a plant, including a bacterial or fungal biotin protein ligase.

60. A method according to claim 58, wherein the test compound is selected by an in silico selection method utilizing the structure of a biotin protein ligase in the absence and presence of biotin to select the compound.

61. A method according to claim 58, wherein the inhibitor has an IC₅₀ of less than 50 μM.

62. A method according to claim 58, wherein the method is used to identify a compound that is a differential inhibitor of a biotin protein ligase from a first organism as compared to a second organism.

63. A method according to claim 62, wherein the first organism is a pathogenic organism and the second organism is a host organism for the pathogenic organism.

64. An inhibitor identified according to the method of claim 58.

65. A biotin protein ligase when used as a target for identifying an inhibitor of biotinylation.

66. A biotin protein ligase according to claim 65, wherein the biotin protein ligase is a biotin protein ligase of a pathogenic organism of a human, animal or plant, including a bacterial or fungal biotin protein ligase.

67. An inhibitor of a biotin protein ligase of a pathogenic organism of a human, animal or plant.

68. An inhibitor according to claim 67, wherein the inhibitor is an anti-bacterial agent and/or an anti-fungal agent.

69. A method of identifying an agent that inhibits growth and/or survival of a pathogenic organism, the method including
identifying an agent that inhibits a biotin protein ligase; and
identifying the agent as an agent that inhibits growth and/or survival of a pathogenic organism by a reduction in the growth and/or survival of the pathogenic organism in the presence of the agent.

70. A method according to claim 69, wherein the pathogenic organism is a bacterium or fungus.

71. A method according to claim 69, wherein the inhibitor has an IC₅₀ for the biotin protein ligase of less than 50 μM.

72. An agent identified according to the method of claim 69.

73. A method of identifying a compound that differentially inhibits biotinylation in a first biological system as compared to inhibition of biotinylation in a second biological system, the method including:
identifying a test compound that inhibits biotinylation in a first biological system;
determining the ability of the test compound to inhibit biotinylation in a second biological system; and
identifying the test compound as a differential inhibitor of biotinylation by a reduction in the biotinylation in the first biological system as compared to the biotinylation in the second biological system.

74. A method according to claim 73, wherein the first biological system is a pathogenic organism and the second biological system is a host organism for the pathogenic organism.

75. A compound identified according to the method of claim 73.

76. A method of inhibiting growth and/or survival of a pathogenic organism, the method including exposing the pathogenic organism to an agent that inhibits biotinylation in the pathogenic organism and thereby inhibit growth and/or survival of the pathogenic organism.

77. A method of preventing and/or treating an infection by a pathogenic organism of a subject, the method including administering to the subject an effective amount of an agent that inhibits a biotin protein ligase of the pathogenic organism and thereby prevent and/or treat an infection by the pathogenic organism.

78. A method according to claim 74, wherein the pathogenic organism is a pathogen of a human, animal or plant subject, including a bacterial or fungal pathogen.
